(19) United States
(12) Patent Application Publication
(10) Pub. No.: US 2022/0152152 A1
(43) Pub. Date: May 19, 2022

(54) PAINLESS NGF FOR FRACTURE REPAIR

(71) Applicant: Steadman Philippon Research Institute, Vail, CO (US)

(72) Inventors: Chelsea BAHNEY, Vail, CO (US); Tejal DESAI, San Francisco, CA (US); Kevin RIVERA, San Francisco, CA (US); Chengbiao WU, La Jolla, CA (US)

(21) Appl. No.: 17/529,180

(22) Filed: Nov. 17, 2021

Related U.S. Application Data
(60) Provisional application No. 63/114,921, filed on Nov. 17, 2020.

(51) Int. Cl. A61K 38/18; A61K 31/727; A61P 19/08

(52) U.S. Cl. CPC A61K 38/185; A61P 19/08; A61K 31/727

(57) ABSTRACT

The present disclosure is related to methods for stimulating bone fracture healing, comprising administering a pharmaceutical composition comprising biomaterial carriers comprising painless nerve growth factor (NGF).

### CROSS-REFERENCE TO RELATED APPLICATIONS

This application claims priority to U.S. Provisional Patent Application No. 63/114,921, filed Nov. 17, 2020, the entire content of which is incorporated by reference herein.

### FIELD OF THE DISCLOSURE

The present disclosure is related to methods for stimulating bone fracture healing, comprising administering a pharmaceutical composition comprising biomaterial carriers comprising painless nerve growth factor (NGF).

### SEQUENCE STATEMENT

The instant application contains a Sequence Listing, which has been submitted electronically in ASCII format and is hereby incorporated by reference in its entirety. Said ASCII copy, created on Nov. 16, 2021, is named 18472-0014USU1_SEQLISTING.txt and is 9 kilobytes in size.

### BACKGROUND OF THE DISCLOSURE

Approximately 15 million fracture injuries occur each year in the United States (US). An estimated 10-15% of fractures within a healthy population result in delayed- or non-union. However, delayed healing rates increase to almost 50% in patients with vascular damage or high co-morbidity burdens such as diabetes, increased age, smoking, and obesity. The current standard of care for delayed healing or non-union is surgical intervention to increase stability or to promote healing through application of bone grafts. However, surgical intervention can result in long-term patient disability and require multiple surgeries to achieve union. Bone autograft remains the gold standard clinical technique for augmenting bone healing in such cases, and while autograft is associated with good healing outcomes, bone harvest increases surgical time and risk of complications by ˜60%, is associated with a high incidence of donor site morbidity, and there is insufficient bone available to fill large defects.

Bone morphogenetic protein (BMP) is the only biologic with FDA approval for use in fracture repair, with “on-label” use only within a very narrow indication window. However, BMP requires surgical implantation and is typically limited to only the most at-risk fractures due to the high cost, limited evidence of clinical efficacy, and risk of severe off-target effects. As such, there exists an unmet clinical need for biologics that could stimulate bone regeneration in a non-surgical delivery platform.

Bone fractures heal primarily through endochondral ossification (EO), a process by which an avascular, aneural cartilage intermediate transforms into vascularized and innervated bone. Despite the importance of endochondral ossification to successful fracture repair, therapeutic approaches to bone regeneration have traditionally focused on promoting intramembranous ossification through the use of BMPs, which forms bone through direct osteoblast differentiation of osteochondroprogenitor.

While it has long been understood that bone is a highly innervated organ system, the functional role of innervation in bone development, homeostasis, and fracture repair is complex and evolving. Nerve growth factor (NGF) was first discovered in the early 1950s and, following decades of research, it is now established for a role in regulating differentiation, growth, survival and plasticity of cholinergic neurons in the central and peripheral systems. NGF exerts its trophic function primarily through binding to the high affinity tropomyosin receptor kinase A (TrkA) receptor. While it has long been understood that bone is a highly innervated organ system, the functional role of innervation in bone development, homeostasis and disease is complex and evolving.

The powerful trophic effect of NGF on the survival and differentiation of sympathetic and sensory neurons has resulted in significant basic science research and a number of clinical trials testing safety and efficacy of NGF for Alzheimer's disease, diabetic neuropathies, chemotherapy-induced and HIV-associated peripheral neuropathies. However, in addition to high affinity binding of NGF to the TrkA receptor, NGF also has low affinity binding to the 75 kDa neurotrophic factor receptor (p75NTR), which recent studies suggest contributes significantly to pain sensation. Unfortunately, serious side effects including back pain and injection site hyperalgesia were noted in dose-dependent clinical studies with NGF and, subsequently, almost all trials have been discontinued.

### BRIEF SUMMARY OF THE DISCLOSURE

In one aspect, the disclosure provides a method for stimulating bone healing in a subject, accelerating bone healing in a subject, and/or improving bone healing in a subject, comprising administering a pharmaceutical composition to the subject, wherein the composition comprises nerve growth factor (NGF).

In another aspect, the disclosure provides a method for stimulating bone healing in a subject, accelerating bone healing in a subject, and/or improving bone healing in a subject, comprising administering a pharmaceutical composition to the subject, wherein the composition comprises biomaterial carriers comprising nerve growth factor (NGF). In certain embodiments, biomaterial carriers “comprising” NGF refers to the NGF being added and essentially adsorbed in the biomaterial carriers, for example, microrods or nanowires. In further embodiments, the biomaterial carriers are frozen or lyophilized for storage stability after the adsorption of NGF. In other embodiments, the biomaterials carriers and NGF are assembled/mixed in a point of care setting.

In one embodiment of a method according to the invention, the bone healing is bone fracture healing.

In one embodiment of a method according to the disclosure, the NGF is a mutant NGF. In another embodiment, the NGF has a mutation at amino acid 100 of the mature NGF protein. In still another embodiment, the (mature) NGF is “painless NGF”, also referred to as NGF^{R100W}. Wild-type NGF amino acid sequence is provided in SEQ ID NO: 1 (human) and 3 (murine). Painless NGF/NGFR^{R100W} amino acid sequence is provided in SEQ ID NO:2 (human) and 4 (murine).

In one embodiment of a method according to the disclosure, a conversion of cartilage to bone is promoted in the subject.

In one embodiment of a method according to the disclosure, the biomaterial carriers are biocompatible. In another embodiment, the biomaterial carriers are biodegradable. In yet another embodiment, the biomaterial carriers are selected from the group consisting of nanowires, nanotubes, nanorods, microwires, microtubes, and microrods. In one embodiment, the biomaterial carriers are microrods. In still another embodiment, the biomaterial carriers are nanowires. In a further embodiment, the nanowires are coated with heparin.

In one embodiment of a method according to the disclosure, the composition is administered by subcutaneous or percutaneous injection. In another embodiment, the administration is local. In still another embodiment, the administration is local to an injury and/or fracture site.

In one embodiment of a method according to the disclosure, bone formation is increased in a fracture.

In another embodiment of a method according to the disclosure, the bone healing is endochondral.

In still another embodiment of a method according to the disclosure, the subject has normal bone healing. In another embodiment, the subject has delayed or non-union bone healing.

In one embodiment of a method according to the disclosure, serum collagen X (Cxm) expression is earlier and/or increased upon administration of the composition.

In another embodiment of a method according to the disclosure, NGF-associated nociception is minimized.

In one embodiment of a method according to the disclosure, the composition is administered during the endochondral or cartilaginous phase of bone healing. In further embodiments, the composition is administered during the chondrogenic phase or during the phase when cartilage is converting to bone. These are not really distinct time points and happen at overlapping times. In another embodiment, the composition is administered between about 1 month and about 4 months post-fracture. In still another embodiment, the composition is administered between about 2 months and about 3 months post-fracture. In further embodiments, a composition according to the disclosure is administered at least a second time, and possibly more times, if healing is delayed/delayed union is observed.

In one embodiment of a method according to the disclosure, the subject has a fracture in a bone that heals through secondary healing or endochondral repair. In another embodiment, the subject has a long bone fracture.

In one embodiment of a method according to the disclosure, newly formed bone contains higher trabecular number, connective density, and/or bone mineral density.

In one embodiment of a method according to the disclosure, cartilage volume in the subject decreases, and bone volume in the subject increases upon administration of the composition. In another embodiment, cartilage volume in the subject decreases by at least about 10%, and bone volume in the subject increases by at least about 10% upon administration of the composition. In still another embodiment, cartilage volume in the subject decreases by at least about 25%, and bone volume in the subject increases by at least about 25% upon administration of the composition.

In an additional embodiment, a method according to the disclosure is useful in osteoporotic indications. In a further embodiment, the osteoporotic indication is osteoporotic fracture. In a still further embodiment, the osteoporotic fracture is atypical femoral neck fracture.

In an additional embodiment, a method according to the disclosure is useful in craniofacial indications. In a further embodiment, the craniofacial indication is selected from the group consisting of craniostenosis/craniosynostosis, cleft palate, mandibular fracture, cranial bone fracture, and cranial bone defect.

In one aspect, the disclosure provides a pharmaceutical composition comprising i) nerve growth factor (NGF) and ii) a pharmaceutically acceptable carrier for use in stimulating bone healing in a subject, accelerating bone healing in a subject, and/or improving bone healing in a subject. In one embodiment, the bone healing is bone fracture healing.

In another aspect, the disclosure provides a pharmaceutical composition comprising

i) biomaterial carriers comprising nerve growth factor (NGF) and ii) a pharmaceutically acceptable carrier for use in stimulating bone healing in a subject, accelerating bone healing in a subject, and/or improving bone healing in a subject. In one embodiment, the bone healing is bone fracture healing.

In one aspect, the disclosure provides a pharmaceutical composition comprising i) nerve growth factor (NGF) and ii) a pharmaceutically acceptable carrier for use in treating bone fracture in a subject.

In another aspect, the disclosure provides a pharmaceutical composition comprising

i) biomaterial carriers comprising nerve growth factor (NGF) and ii) a pharmaceutically acceptable carrier for use in treating bone fracture in a subject.

In one embodiment of a pharmaceutical composition of the disclosure, the NGF is a mutant NGF. In another embodiment, the NGF has a mutation at amino acid 100 of the mature NGF protein. In still another embodiment, the mature NGF is NGF^{R100W}.

In one embodiment of a pharmaceutical composition of the disclosure, the biomaterial carriers are biocompatible. In another embodiment, the biomaterial carriers are biodegradable. In still another embodiment, the biomaterial carriers are selected from the group consisting of nanowires, nanotubes, nanorods, microwires, microtubes, and microrods. In another embodiment, the biomaterial carriers are microrods. In yet another embodiment, the biomaterial carriers are nanowires. In a further embodiment, the nanowires are coated with heparin.

In one embodiment, a pharmaceutical composition according to the disclosure is administered to a subject by subcutaneous or percutaneous injection. In another embodiment, the administration is local. In still another embodiment, the administration is local to an injury and/or fracture site.

In one embodiment of a composition according to the disclosure, the bone healing is endochondral. In another embodiment, the subject has normal bone healing. In yet another embodiment, the subject has delayed or non-union bone healing.

In one embodiment, a pharmaceutical composition according to the disclosure is administered during the endochondral/cartilaginous phase of bone healing. In another embodiment, the composition is administered between about 1 month and about 4 months post-fracture. In still another embodiment, the composition is administered between about 2 months and about 3 months post-fracture. In further embodiments, a composition according to the disclosure is administered at least a second time, if healing is delayed/delayed union is observed.

In one embodiment of a composition according to the disclosure, the subject has a fracture in a bone that heals through secondary healing or endochondral repair. In another embodiment, the subject has a long bone fracture.

In an additional embodiment, a composition according to the disclosure is useful in osteoporotic indications. In a further embodiment, the osteoporotic indication is osteoporotic fracture. In a still further embodiment, the osteoporotic fracture is atypical femoral neck fracture.

In an additional embodiment, a composition according to the disclosure is useful in craniofacial indications. In a further embodiment, the craniofacial indication is selected from the group consisting of craniostenosis/craniosynostosis, cleft palate, mandibular fracture, cranial bone fracture, and cranial bone defect.

Other embodiments will become apparent from a review of the ensuing detailed description.

### BRIEF DESCRIPTION OF THE FIGURES

FIG. 1 shows a schematic illustration of the phases and timeline for endochondral fracture repair in a murine model of tibia fracture. NGF or painless NGF can work by accelerating intramembranous bone formation or endochondral bone formation by accelerating cartilage-to-bone transformation (purple). Timeline is for murine healing. Human scale is ˜4-6 times as long for normal healing.

FIGS. 2A and 2B show that painless NGF does not induce thermal hyperalgesia. (FIG. 2A) Latencies of paw withdrawal at 55° C. (Mean±SEM, ANOVA followed by Dunnett's multiple comparisons against baseline); (FIG. 2B) Reduction in nociceptive threshold from the baseline.

FIGS. 3A-3L show that NGF^{R100W} promotes regeneration of paw skin sensory nerves in CMT2B mutant mice. CMT2B mutant mice were injected twice per week intradermally with either WT NGF (ipsilateral, FIG. 3B, contralateral, FIG. 3E) or NGF^{R100W} (ipsilateral, FIG. 3C, contralateral, FIG. 3F), or left untreated (FIG. 3A). (FIG. 3D) relative density of IENFs (mean grey value/unit area) for stained sections in bar graph form. 6 weeks following injections mice were sacrificed and hindpaw skin was extracted, fixed, sectioned, and stained for PGP9.5. Epi: epidermis; SNP: subepidermal neural plexus; red arrow heads: IENF (intra-epidermal nerve fibers). One-Way ANOVA Mean±SEM, *** p<0.005; **** p<0.001 (Dunnett's test). Painless NGF shows similar in vitro stimulation of endochondral ossification in ATDC5 cells by painless and wild type NGF. Chondrogenic cells were treated with 20 g/mL of NGF or NGF^{R100W} and gene expression was measured by qRT-PCR 1- or 24-hours following treatment for (FIG. 3G Vegf, (FIG. 3H) Axin 2, and (FIG. 3I) lhh. (N=3, **=p<0.01, *** p<0.005 by Tukey HSD). NGF^{R100W} exhibits trophic activity in chondrocytes. NGF^{R100W} (green) showed enhanced bioactivity relative to NGF^{WT} (blue) at a concentration of 0.2 mg through upregulated endochondral (Indian hedgehog, lhh) and osteogenic (alkaline phosphatase and osteocalcin) genes. ATDC5 chondrogenic cells were cultured for 7 days in chondrogenic media prior to adding 0.2 or 20 mg of NGF or NGF^{R100W} for 24 hours. mRNA was isolated and qRT-PCR for many genes including (FIG. 3J Indian hedgehog, ihh, (FIG. 3K) alkaline phosphatase, alp, and (FIG. 3L) osteocalcin. *p<0.05, **p<0.01, **p<0.005

FIGS. 4A-4H show endogenous expression of Nerve growth factor (NGF) and its receptor Tropomyosin receptor kinase A (TRKA) within fracture callus during endochondral repair. (FIG. 4A) A gross fluoroscope image of the entire tibia with the red frame indicating the mid diaphyseal, unstabilized bone fracture. (FIG. 4B) Representative image of HBQ stained section of tibia fracture callus 14 days post-fracture (p.f.), (n=4). Scale bar: 1 mm (FIG. 4C) Fluorescence image NGF-eGFP with DAPI of chondro-osseous transition zone (TZ) 14 days p.f. (n=4). Scale bar: 200 μm (FIG. 4D) Brightfield image of X-GAL stained callus 14 days p.f. (n=4). Arrows indicate additional areas of LACZ+ cells within callus. Scale bar: 500 μm (FIG. 4E) Higher magnification image of TZ within fracture callus. (FIG. 4F) Higher magnification image of cortical bone shows no staining. (FIGS. 4E, 4F) Scale bars: 200 μm (FIG. 4G) Relative expression (2−ΔCT) normalized to Gapdh of Ngf and (FIG. 4H) TrkA harvested from fracture callus at 7, 10, and 14 days p.f. Error bars represent SEM. *p<0.05; determined by one-way ANOVA with Tukey's multiple comparison test.

FIGS. 5A-5D shows that local β-NGF injections during hypertrophic cartilage phase promotes osteogenic marker expression. (FIG. 5A) Timeline schematic of fracture and three daily injections 0.5 μg β-NGF vs control (media injected) starting at 4 days post-fracture. (FIG. 5B) Expression levels of selected osteogenic and angiogenic markers from whole-callus tissue harvested 24 h after final injection. (FIG. 5C) Timeline of fracture and three daily injections 0.5 μg β-NGF vs control) starting at 7 days post-fracture. (FIG. 5D) Expression levels of osteogenic and angiogenic markers from whole-callus tissue harvested 24 h after final injection. All expression levels are relative to Gapdh; calculated by 2−ΔCT. Error bars represent SEM. *p<0.05, **p<0.01; determined by 2-tailed t test.

FIGS. 6A-6C show that recombinant human β-NGF (β-NGF) promotes gene expression profile for endochondral bone formation. (FIG. 6A) Volcano plot of differentially expressed genes in hypertrophic cartilage stimulated with β-NGF. Threshold set to ≥1 log 2 fold change (equal to ≥two-fold change), endochondral ossification-associated markers are denoted (n=3). (FIG. 6B) Upregulated molecular function categories generated by EnrichR (maayanlab.cloud/Enrichr/), gene ontology terms are sorted by p values with corresponding adjusted p value and odds ratio (FIG. 6C) Heatmap depicting relative expression of genes associated with Wnt activation, PDGF binding, and integrin binding. p<0.05, Benjamini-Hochberg method. The first panel was generated by the R package ggplot2 (version 3.2.1) (Ginestet 2020 J Bone Miner Res 35:143-154). The third panel was generated by Complexheatmap (version 2.0) on Bioconductor (Bioconductor.org).

FIG. 7 shows a table listing the expression levels of genes of interest from β-NGF vs non-stimulated cartilage explants, generated by RNA sequencing.

FIGS. 8A and 8B show that enrichment analysis of β-NGF stimulated hypertrophic cartilage explants. (FIG. 8A) Principal component analysis (PCA) for each biological replicate of β-NGF and non-stimulated controls. Gene ontology (GO) categorical grids for (FIG. 8B) downregulated molecular functions. To generate data, cartilaginous tissue was excised from tibia fracture 7 days post-fracture and cultured to hypertrophy for 7 days then stimulated with or without recombinant human β-NGF. Samples were collected after 24 hours for RNA-seq analysis (n=3). GO terms, p values, and odds ratios were generated and computed by Enrichr. GO terms are sorted by p value, adjusted p value determined by Benjamini-Hochberg method.

FIGS. 9A-9K show that local injections of β-NGF induce Wnt activation in the TZ and nominal increase of endothelial cell infiltration of cartilage callous. (FIG. 9A) Timeline schematic of fracture and subsequent daily injections of β-NGF. (FIG. 9B) Representative image of HBQ stained section of the chondro-osseous transition zone (TZ) from control group (media injections) with (FIG. 9C) a corresponding fluorescent DAPI-stained image of adjacent slide. (FIG. 9D) Image of HBQ stained TZ from β-NGF treated mice with corresponding (FIG. 9E) fluorescent DAPI stained image of an adjacent slide (FIG. 9F) Quantification of Axin2-eGFP presence within TZ of fracture callus as percentage (%) of area. Images of (FIG. 9G) HBQ stained section of cartilage tissue within fracture callus from control group and corresponding image (FIG. 9H) of Anti-CD31 Diaminobenzidine (DAB) stained section. (FIG. 9I) HBQ stained section from β-NGF treated group and corresponding (FIG. 9J) CD31-DAB stained section. (FIG. 9K) Quantification of DAB stain within cartilaginous tissue as percentage (%) of area. All scale bars=500 μm. Error bars represent SEM. **p<0.01; determined by 2-tailed t test.

FIG. 10A-10I show that local injections of β-NGF result in less cartilage and more bone. Representative images of HBQ stained section of fracture callus from (FIG. 10A) control group and (FIG. 10B) β-NGF group, 14 days post fracture. Scale bar: 500 μm. Quantification of cartilage volume in both treatment groups, shown as (FIG. 10C) absolute volume and as (FIG. 10D) percent composition of the total callus volume. Quantification of bone volume in both treatment groups shown as (FIG. 10E) absolute volume and (FIG. 10F) percent composition. Quantification of (FIG. 10G) whole-callus volume (FIG. 10H) bone marrow and (FIG. 10I) fibrous tissue. All measured by stereology. Error bars represent SEM. *p<0.05; **p<0.01 determined by 2-tailed t test.

FIGS. 11A-11F show that local injections of β-NGF result in highly connected trabecular bone. μCT images of tibias from (FIG. 11A) control and (FIG. 11B) β-NGF treated mice, 14 days post fracture. Scale bar=1 mm. Quantification of (FIG. 11C) trabecular spacing (FIG. 11D) trabecular number (FIG. 11E) trabecular connective density and (FIG. 11F) bone mineral density. Error bars represent SEM. *p<0.05; **p<0.01 determined by 2-tailed t test.

FIGS. 12A-12C show further μCT analysis of trabecular bone within fracture callus. Local injections of media (control) or 0.5 μg β-NGF were administered once daily at 7, 8, and 9 days post-fracture (p.f.), tibias were then harvested 14 days p.f. for μCT analysis. Quantification of (FIG. 12A) bone volume as percent composition (FIG. 12B) average trabecular thickness and (FIG. 12C) tissue mineral density is shown for control (n=10) and β-NGF (n=5) treated mice. #=p<0.1, determined by 2-tailed t test.

FIG. 13 shows a schematic of PEGDMA microrod fabrication via photolithography.

FIGS. 14A-140 show that lyophilized PEGDMA microrods are readily protein loaded. (FIG. 14A) Loading efficiency significantly increases with increasing concentration of PEGDMA (% PEGDMA v/v). Data shown as means with error bars representing SEM. *p<0.05, **p<0.01, ***p<0.001 determined by ANOVA with Tukey's post hoc test for multiple comparisons (n=3) (FIG. 14B) DAPI-loaded 90% PEGDMA microrods, scale bar=25 μm. (FIG. 14C) Fluorescent micrographs of FITC-BSA loaded in 90% PEGDMA microrods taken after 0 mins (top) and after 60 mins of incubation at room temperature (bottom), scale bars=50 μm. (FIG. 14D Loading efficiency of β-NGF onto 90% PEGDMA (v/v) microrods, measured by microBCA (n=3). Data shown as mean with error bar representing SEM.

FIGS. 15A-15C show β-NGF loaded onto PEGDMA microrods retain bioactivity. (FIG. 15A) Relative fold change in TF-1 cell proliferation (day 4 vs day 0) for each experimental group. *p<0.05 determined by ANOVA with Tukey's post hoc test for multiple comparisons (n=4). (FIG. 15B) Cumulative mass (in ng) and (FIG. 15C) daily mass (in ng) of beta NGF released from 90% PEGDMA microrods over a 7-day period shown in hours (n=4). All data shown as means with error bars representing SEM.

FIGS. 16A-16G show localization of PEGDMA microrods within tibial fracture calluses. Representative micrographs of (FIG. 16A) low and (FIG. 16B) high magnification of HBQ-stained fracture calluses 5 days post-microrod injection. Representative micrographs of (FIG. 16C) low and (FIG. 16D) high magnification of fracture calluses 7 days post-microrod injection. Arrows indicate PEGDMA microrods within calluses. (FIGS. 16A, 16C) Scale bars=1 mm, (FIGS. 16B, 16D) Scale bars=100 μm. (FIG. 16E) Representative micrograph of fracture callus 14 days post-injection, scale bar=1 mm. (FIG. 16F Three-point fracture device used to create closed non-stabilized fractures on mouse tibia. (FIG. 16G) Gross fluoroscope image of the entire tibia with yellow frame indicating the mid diaphyseal bone fracture.

FIGS. 17A-17J show microCT analysis of newly formed bone within fracture calluses. Representative three-dimensional images of tibial fracture calluses from mice treated 14 days post-fracture with (FIG. 17A) saline as controls (FIG. 17B) single dose of β-NGF (2000 ng) (FIG. 17C) non-loaded PEGDMA microrods and (FIG. 17D) PEGDMA microrods loaded with β-NGF (18 ng). Scale bars=1 mm. Quantification of (FIG. 17E) bone volume fraction (FIG. 17F) trabecular connective density and (FIG. 17G) bone mineral density. Error bars represent SEM, *p<0.05, **p<0.01 determined by ANOVA with Tukey's post hoc test for multiple comparisons. MicroCT analysis of trabecular bone within fracture callus. Quantification of (FIG. 17H trabecular separation (FIG. 17I) trabecular number, and (FIG. 17J) trabecular thickness in mice treated with saline (as control), single dose of β-NGF (2000 ng), non-loaded PEGDMA microrods and, PEGDMA microrods loaded with β-NGF (18 ng). Error bars represent SEM, non-significance determined by ANOVA with Tukey's post hoc test for multiple comparisons.

FIGS. 18A-18J show the results of single injections of PEGDMA microrods loaded with β-NGF promote endochondral bone repair. Representative micrographs of HBQ-stained fracture calluses from mice 14 days post-fracture treated with (FIG. 18A) saline as controls (FIG. 18B) single dose of β-NGF (2000 ng). (FIG. 18C) non-loaded PEGDMA microrods and (FIG. 18D) PEGDMA microrods loaded with β-NGF (18 ng). Left column scale bars=2 mm, middle and right column scale bars=500 μm. Quantification of (FIG. 18E) cartilage volume and (FIG. 18F) bone volume, both given as percent composition of fracture callus. Histomorphometric analysis of fracture calluses. Quantification of (FIG. 18G total callus volume (FIG. 18H) fibrous tissue absolute volume (FIG. 18I) cartilage absolute volume and (FIG. 18J) bone absolute volume in mice treated with saline (as control), single dose of β-NGF (2000 ng), non-loaded PEGDMA microrods and, PEGDMA microrods loaded with β-NGF (18 ng). Error bars represent SEM, *p<0.05 determined by ANOVA with Tukey's post hoc test for multiple comparisons.

FIG. 19 shows the polymeric nanowire fabrication technique. Nanowires are fabricated using an anodic aluminum oxide (AAO) template with 200 nm pores. The polymeric materials are heated past their melting temperatures, causing nanowire formation via capillary action. After nanowire solidification, the membrane is detached and etched to release the nanowires. Nanowires are 200 nm wide, while length is dependent upon polymer film thickness, with tunable lengths ranging from 2-20 microns.

FIGS. 20A-20C show the layer-by-layer assembly of NGF-functionalized nanowires. (FIG. 20A) PCL nanowires can be functionalized with charged polymers for layer-by-layer (LbL) assembly and NGF loading onto heparin. (FIG. 20B) Zeta potential demonstrates deposition of chitosan and heparin onto nanowires. (FIG. 20C) Hypothesized relationship of LbL layers and NGF release rates.

FIGS. 21A-21D show that the biomaterial platforms can be tuned for sustained release of bioactive NGF. (FIG. 21A) Adsorption efficiency of NGF loaded into PEGDM microrods was calculated using a mBCA assay. (FIG. 21B) With ˜20 ng of NGF loaded into the 90% PEGDM microrods, protein release was characterized over the time course of 7-days. (FIG. 21C) NGF was loaded with 75% efficiency onto nanowires for approximately 5 μg NGF/mg nanowires, followed by a single layer of chitosan. Sustained first order NGF release was observed over 8 days (R2=0.999). (FIG. 21D) Bioactivity of the NGF released from PEGDM microrods was confirmed using the TF1 cell proliferation assay. *p<0.05, **p<0.01

FIGS. 22A-22C show that accelerated healing could be detected through novel biomarker where peak in CXM curve would shift left in therapy promotes endochondral repair. (FIG. 22A) serum Cxm biomarker during fracture repair in male and female mice (n>8/gender), (FIG. 22B) qRT-PCR of colXa1 gene expression in the fracture callus (n=5/sex), (FIG. 22C) CoIX immunohisto-chemistry (brown) in transition zone.

### DETAILED DESCRIPTION

Before the present methods are described, it is to be understood that this disclosure is not limited to particular methods, and experimental conditions described, as such methods and conditions may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, preferred methods and materials are now described. All publications mentioned herein are incorporated herein by reference in their entirety.

### Definitions

The term “bone fracture”, as used herein, refers to a partial or complete break in the continuity of a bone. The fracture of the bone may be closed or open (compound). The fracture of the bone may be displaced. Stress fractures, also referred to as hairline fractures, are also bone fractures. Bone fractures may be transverse, spiral, oblique, compression, comminuted, avulsion, impacted, etc. A bone fracture may be diagnosed vie X-ray imaging, magnetic resonance imaging (MRI), bone scan, computed tomography scan (CT/CAT scan), or other known methods.

In specific embodiments of the methods, uses, and compositions according to the disclosure, the fracture is a fracture in any bone that heals through secondary healing or endochondral repair. In a further embodiment, the fracture is a long bone fracture.

Bone fracture treatment traditionally depends on the location, type, and severity of fracture. Treatment may include repositioning the bone, followed by immobilization via a plaster or fiberglass cast, repositioning the bone, followed by partial immobilization via a functional cast or brace, support/partial immobilization via splint, open reduction with internal fixation, open reduction with external fixation, and other methods known to the clinician.

The methods and compositions according to the disclosure can additionally be useful in osteoporotic indications. One osteoporotic indication is osteoporotic fracture. Osteoporotic fracture may, for example, be atypical femoral neck fracture.

The methods and compositions according to the disclosure can additionally be useful in craniofacial indications. The craniofacial indication may, for example, be selected from the group consisting of craniostenosis/craniosynostosis, cleft palate, mandibular fracture, cranial bone fracture, and cranial bone defect.

By the phrase “therapeutically effective amount” is meant an amount that produces the desired effect for which it is administered. The exact amount will depend on the purpose of the treatment, and will be ascertainable by one skilled in the art using known techniques (see, for example, Lloyd (1999), The Art, Science and Technology of Pharmaceutical Compounding).

As used herein, the term “subject” refers to an animal, preferably a mammal, more preferably a human. As such, subjects of the disclosure may include, but are not limited to, humans and other primates, such as chimpanzees and other apes and monkey species; farm animals such as cattle, sheep, pigs, goats, and horses; domestic mammals such as dogs and cats; laboratory animals including rodents such as mice, rats, and guinea pigs; birds, including domestic, wild, and game birds such as chickens, turkeys, and other gallinaceous birds, ducks, geese, and the like. In certain embodiments, the subject is a human. The term includes mammalian, including human, subjects having a bone fracture.

As used herein, the terms “treat”, “treating”, or “treatment” refer to the healing of a bone fracture in a subject in need thereof. The terms include healing of the actual fracture and may additionally or alternatively include ameliorating a symptom associated with the bone fracture, for example, pain, inflammation, reduced mobility, etc.

### Fracture Healing

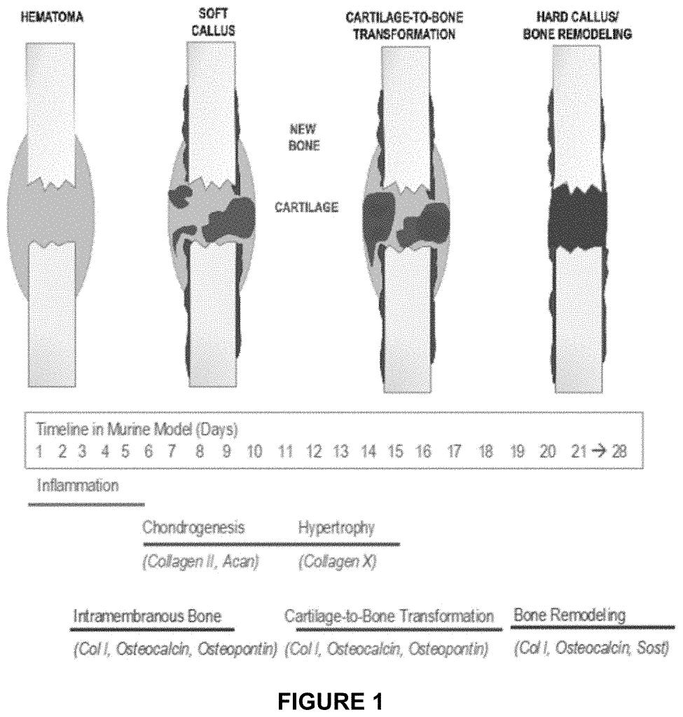

Fracture healing is a dynamic regenerative process that can fully restore the native form and function of an injured bone. The majority of fractures heal indirectly through a cartilage intermediate in a process that draws parallels to endochondral ossification (EO) in bone development (FIG. 1). Following a long bone fracture, a hematoma forms to stop the bleeding, contain debris, and trigger a pro-inflammatory response that initiates repair (Kolar, et al., 2010, Tissue Engineering, Part B, Reviews 16:427-434; Xing, et al., 2010, J Orthopaedic Res 28:1000-1006). Periosteal and endosteal progenitor cells undergo osteogenic differentiation to form new bone along the existing bone ends adjacent to the fracture through intramembranous ossification (Colnot, et al., 2009, J Bone Miner Res 24:274-282). In the fracture gap, periosteal progenitor cells differentiate into chondrocytes and generate a provisional cartilaginous matrix that gives rise to bone indirectly by EO (Le, et al., 2001, J Orthopaed Res 19:78-84). The cartilage callus matures to bone through transformation of chondrocytes into osteoblasts (Hu, et al., 2017, Development 144:221-234; Zhou, et al., 2014, PloS genetics 10:e1004820; Yang, et al., 2014, PNAS USA 1302703111). The newly formed trabecular bone then remodels into cortical bone (Drissi, et al., 2016, J Cellular Biochem 117:1753-1756).

Bone fracture healing comprises an inflammatory phase (fracture hematoma formation), a repairing/reparative phase (during which the body develops cartilage and tissue in and around the fracture site, calluses grow and stabilize the fracture, and trabecular bone replaces the tissue callus), and a bone remodeling phase (during which spongy bone is replaced with solid bone). During the inflammation stage of fracture healing/repair, the biological processes hematoma, inflammation, and recruitment of mesenchymal stem cells take place. During the cartilage formation and periostal response stage of fracture healing/repair, the biological processes chondrogenesis and endochondral ossification, cell proliferation in intramembranous ossification, vascular in-growth, and neo-angiogenesis occur. During the cartilage resorption and primary bone formation stage of fracture healing/repair, the biological processes active osteogenesis, bone cell recruitment and woven bone formation, chondrocyte apoptosis and matrix proteolysis, osteoclast recruitment and cartilage resorption, and neo-angiogenesis take place. Finally, during the secondary bone formation and remodeling stage of fracture healing/repair, the biological processes bone remodeling coupled with osteoblast activity and establishment of marrow occur (Al-Aql, et al., 2008, J Dent Res 87(2):107-118).

In certain embodiments, a subject does not experience normal fracture healing. In specific embodiments, such a subject may experience mal-union (bone fracture healing in a deformed, non-anatomical position; can be functionally and/or cosmetically unacceptable), delayed (significantly longer, for example, about twice as long as expected/average fracture healing time), or non-union (failure of the broken bones to unite) fracture healing.

Average fracture healing time may differ depending on the specific bone and/or the level of blood supply in the area of the bone. For example, fractures present in areas of high blood supply, like the spine, the wrist, etc., heal earlier than fractures present in areas of low blood supply, like the scaphoid (wrist bone), the tibia (leg bone), etc. Average fracture healing time may also vary depending on the age of the subject, where the same bone fracture may take twice as long to heal in an elderly person as in a child. The clinician is aware of the general ranges of healing time and can identify delayed fracture healing in a subject.

Factors that can delay bone fracture healing include, without limitation, glucocorticoid excess, diabetes, hormonal imbalance, vitamin D deficiency, severe anemia, injury, infection, neoplasm, metabolic diseases, smoking, excessive mobility at the fracture site, separation of the bone ends, and dilution by the synovial fluid.

In one aspect, the disclosure provides a method for stimulating bone fracture healing in a subject, comprising administering a pharmaceutical composition to the subject, wherein the composition comprises biomaterial carriers comprising nerve growth factor (NGF). In one embodiment, stimulating bone fracture healing comprises converting cartilage to bone faster and improving quality of bone and/or forming better bone structure.

In another aspect, the disclosure provides a method for accelerating bone fracture healing in a subject, comprising administering a pharmaceutical composition to the subject, wherein the composition comprises biomaterial carriers comprising nerve growth factor (NGF). In one embodiment, accelerating bone fracture healing comprises converting cartilage to bone faster.

In another aspect, the disclosure provides a method for improving bone fracture healing in a subject, comprising administering a pharmaceutical composition to the subject, wherein the composition comprises biomaterial carriers comprising nerve growth factor (NGF). In one embodiment, improving bone fracture healing comprises improving quality of bone and/or forming better bone structure.

In another aspect, the disclosure provides a method for treating a subject having a bone fracture, comprising administering to the subject a pharmaceutical composition according to the disclosure. In certain embodiments of the methods according to the disclosure, bone formation is increased in the fracture.

In specific embodiments of the methods, uses, and compositions according to the disclosure, the fracture healing is endochondral. The fracture healing stimulated, accelerated, and/or improved is during endochondral ossification. Thus, in certain embodiments, the composition comprising biomaterial carriers comprising at least one bioactive compound is administered during the endochondral/cartilaginous phase of fracture healing. The clinician uses experienced judgement, reduction in patient-reported pain, increased stiffness/mobility of the fracture, and a “hazy” appearance in the X-ray to estimate when the soft callus phase is peaking, for administration of a composition according to the disclosure, in certain embodiments. In further embodiments, the composition is administered between about 1 month and about 4 months post-fracture. In still another embodiment, the composition is administered between about 2 months and about 3 months post-fracture. In further embodiments, a composition according to the disclosure is administered at least a second time, if healing is delayed/delayed union is observed.

### Painless NGF

In one embodiment of the methods, compositions, and uses of the disclosure, the bioactive compound is NGF. In another embodiment of the methods, compositions, and uses of the disclosure, the bioactive compound is mutant NGF. In still another embodiment of the methods, compositions, and uses of the disclosure, the bioactive compound is NGF^{R100W}, or painless NGF. Thus, in embodiments in which mutant NGF, specifically, NGF^{R100W}, is comprised in the biomaterial carriers administered to the subject, NGF-associated nociception is minimized.

The recent discovery of a naturally occurring point mutation leading to a change from arginine to tryptophan at residue 100 in the mature NGFβ sequence (NGF^{R100W}) in patients with hereditary sensory and autonomic neuropathy type V suggests that it is possible to uncouple trophic effects from nociceptive function (Sung, et al., 2018, J Neurosci 38:3394-3413). Similar to the wild-type NGF, the NGF^{R100W} mutant is capable of binding to and activating the TrkA receptor and its downstream signaling pathways to support the trophic functions associated with NGF. However, NGF^{R100W} fails to engage the p75NTR signaling pathways, eliminating thermal and mechanical acute hyperalgesia. Other mutations at the R100 position (NGFR100E and NGFP61SR100E), similarly, find effective binding of these mutant NGFs to TrkA, with abolished NGF binding to p75NTR (Capsoni, et al., 2011, PloS one 6:e17321; Covaceuszach, et al., 2010, Biochem biophys res comm 391:824-829). Using a non-genetic approach, studies have found that injecting a p75NTR neutralizing antibody blocked NGF-induced hyperalgesia while enabling NGF-mediated sensitization of action potentials in sensory neurons (Watanabe, et al., 2008, J Neurosci Res 86:3566-3574; Zhang, et al., 2004, Neurosci Lett 366:187-192).

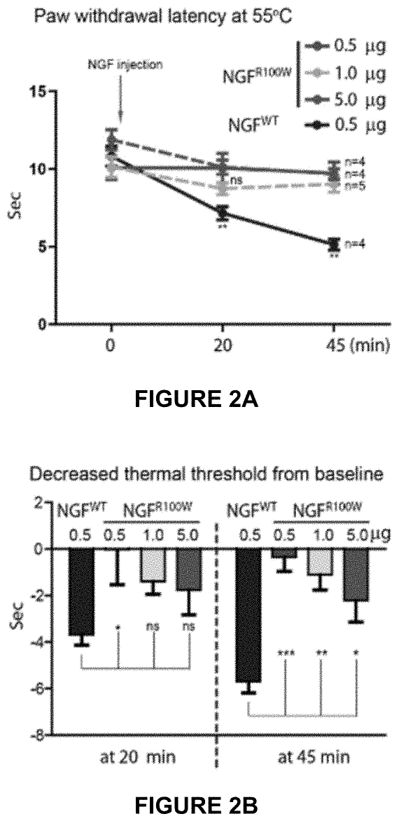

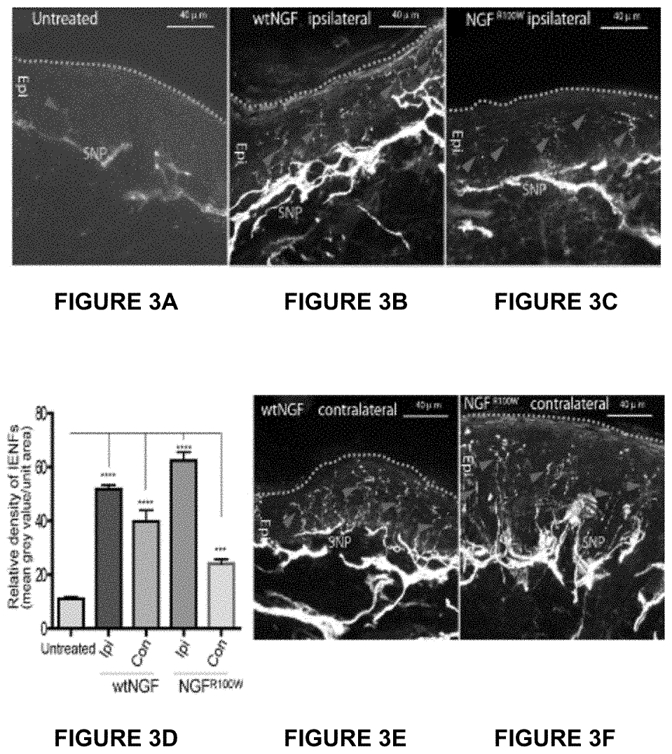

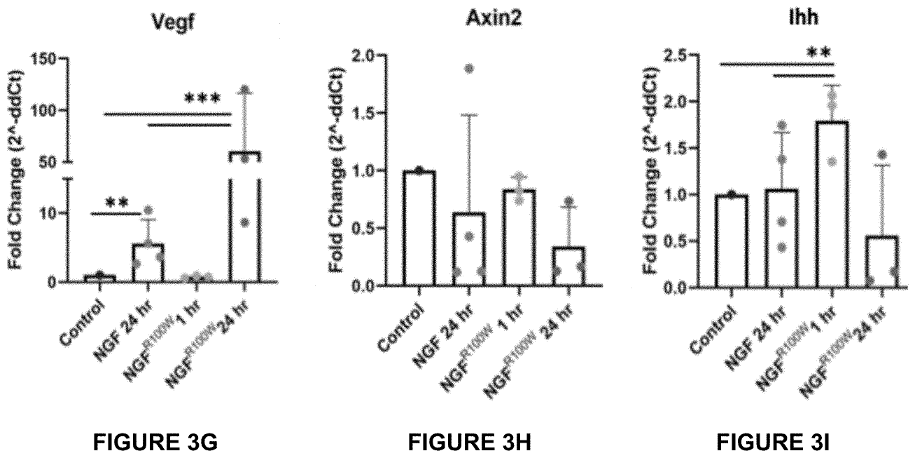

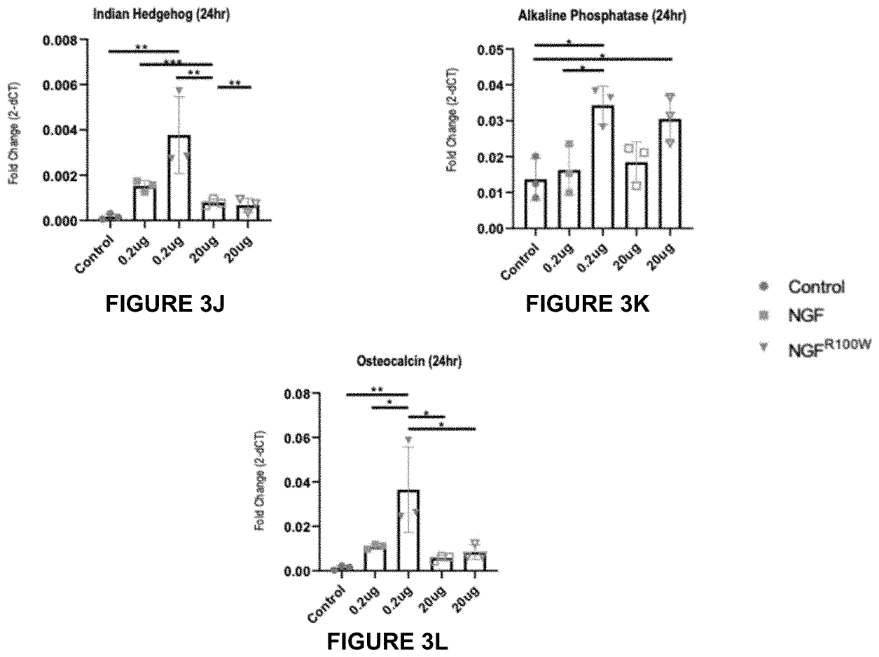

FIGS. 2A and 2B show that the painless is truly painless. FIGS. 3A-3F shows that, despite not inducing pain, painless NGF is truly regenerative.

The evaluation of painless NGF (NGF^{R100W}) as a novel biologic for stimulating fracture repair is disclosed herein.

### Biomaterial Carriers

In one embodiment of a method, use, or composition according to the disclosure, the biomaterial carriers are biocompatible. As used herein, the term “biocompatible” implies compatibility with a living system or living tissue, e.g., an animal or animal tissue, e.g. a human or human tissue, not being toxic, injurious, or physiologically reactive and/or causing a harmful immunological reaction.

In another embodiment of a method, use, or composition according to the disclosure, the biomaterial carriers are biodegradable. As used herein, the term “biodegradable” implies capability of being broken down, especially into innocuous products, by a natural system or natural components thereof, for example, in an animal subject, for example, in a human subject.

In yet another embodiment, the architecture of the biomaterial carriers is selected from the group consisting of nanowires, nanotubes, nanorods, microwires, microtubes, and microrods. Biomaterial carriers having a rod-like shape (i.e., tubes, rods, wires) benefit from their high aspect ratio.

In some embodiments, the biomaterial carriers are coated with a tissue-compatible substance. In specific embodiments, the tissue-compatible substance is an anti-inflammatory and/or an anticoagulant substance. In additional specific embodiments, the tissue-compatible substance is chitosan. In further specific embodiments, the tissue-compatible substance is a substance that delays and/or prolongs the release of growth factors. In still further embodiments, the tissue-compatible substance is selected from heparin, heparin sulfate, hyaluronic acid, and heparin+hyaluronic acid in combination.

The development of translationally relevant micro- and nanotechnology platforms for local and controlled delivery of painless NGF is disclosed herein. An injectable, bioinspired drug delivery platform based on polycaprolactone (PCL) polymers fabricated into nanowires has been used to modulate local receptor-ligand interactions for cytokine-mediated disease and offer improved pharmacokinetics compared to systemic cytokine therapy (Zamecnik, et al., 2017, ACS Nano 11:11433-11440). Upon injection, these nanowires self-assemble into a loose network and can stay in place for at least 9 days in a subcutaneous mouse model.

These nanowires, nanotubes, nanorods, microwires, microtubes, and microrods were designed to enable a non-surgical delivery technology with high clinical relevance. Due to their small size, they can be easily injected for percutaneous delivery to the fracture sight and should not interfere with the normal healing process.

In other embodiments, the nanowires, nanotubes, nanorods, microwires, microtubes, and microrods are not easily phagocytized by macrophages due to their shape(s) and reduce fibrotic tissue formation as a consequence.

In further embodiments, the biomaterial carriers comprising the bioactive compound stabilize the compound. For example, the microrod or nanowire may stabilize the NGF or painless NGF they comprise. This stabilization is likely related to the biomaterial protecting the compound from degradation. Furthermore, the controlled release provided by the biomaterial carrier results in a requirement for less bioactive compound, as the latter is provided slowly and is not quickly degraded. Thus, in certain embodiments, less NGF or painless NGF is required to achieve its biological activity when it is comprised in the nanowire or microrod than when it is administered on its own.

### Nanowires

In certain embodiments of the methods, uses, and compositions of the disclosure, the biomaterial carriers comprise individual polymeric nanowires (“nanowires”). In further embodiments, the nanowires include at least one bioactive compound, for example, painless NGF. The term “individual polymeric nanowires” as used herein refers to a composition that includes discrete, free-floating polymeric nanowires in a fluidic solution where each individual nanowire is not joined to any other nanowire in the solution. In particular, individual polymeric nanowires of the subject compositions are not connected together to each other (e.g., covalently bonded) or affixed to a common substrate. The individual polymeric nanowires are formed in a vertical array of parallel pores of a template structure and are removed, so that there is no permanent connection between each polymeric nanowire or a bond between the polymeric nanowires and a substrate.

### Nanowire Platform for Controlled and Local NGF Delivery

In addition to validating painless NGF (NGF^{R100W}) as a novel biologic for stimulating fracture repair, a translationally relevant nanotechnology platform for local and controlled delivery is disclosed herein. To accomplish this, a bioinspired drug delivery platform based on polycaprolactone (PCL) polymers fabricated into nanowires is tuned. Such an injectable nanomaterial can be used to modulate local receptor-ligand interactions for cytokine-mediated disease and offer improved pharmacokinetics compared to systemic cytokine therapy (Kronenberg, 2003, Nature 423:332-336). Upon injection, these nanowires self-assemble into a loose network, and they can stay in place for at least 9 days in a subcutaneous mouse model (data not shown). PCL was chosen as the base polymer, because the highly tunable, biodegradable thermoplastic is amendable to nanofabrication techniques, and the material already has FDA approval in sutures (Bahney, et al., 2014 J Bone Mineral Res 29(5) doi:10.1002/jbmr.2148). Furthermore, PCL has been shown to elicit minimal local immune response when used in larger medical implants (Shinoda, et al., 2011 J Neuroscience 31(19):7145-7155), in contrast to polypropylene and polylactide-co-glycolide (PLGA), which have demonstrated significant non-specific inflammatory responses (Hopkins and Slack, 1984, Neuroscience 13(3):951-956; Sonnet, et al., 2013, J Orthopaedic Res 31:1597-1604; Stukel, et al., 2015, J Biomed Materials Res Part A 103:604-613).

The PCL nanowire platform technology is functionalized for bioactivity through the attachment of NGF^{R100W} using a layer-by-layer (LbL) electrostatic assembly approach (Kronenberg, 2003, Nature 423:332-336). The PCL nanowires bear a strong negative charge capitalized upon to electrostatically assemble chitosan (positive charge) and heparin (negative charge) onto the nanowires. In addition to its positive charge, chitosan has antimicrobial properties and, therefore, has been used successfully in medical device coatings and drug delivery systems (Olabisi, et al., 2010, Tissue Engineering Part A 16:3727-3736; Xu, et al., 2017, Biomaterials 147:1-13; Rot, et al., 2014, Developmental Cell 31:159-170). Heparin was chosen for its ability to bind to and stabilize a variety of growth factors, including NGF, with moderate to high affinities and serves a modular means of loading growth factor cargo onto nanowires.

The nanowires of the methods and compositions according to the disclosure are designed to enable a non-surgical delivery technology with high clinical relevance. In certain embodiments, the PCL nanowires are about 200 nm in diameter and about 15-20 μm in length. Due to their small size, they can be easily injected for percutaneous delivery to the fracture site and will not interfere with the normal healing process, as some current materials have been shown to do (Parekh, et al., 2011, Biomaterials 32:2256-2264).

The at least one bioactive compound may be absorbed into pores of the polymeric nanowires or may be affixed to a surface of the polymeric nanowire, such as by non-covalent interactions (e.g., ionic forces, dipole-dipole interactions, hydrogen bonding) or by one or more covalent bonds. The subject polymeric nanowires are configured to deliver the at least one bioactive compound to a target site, such as by injecting the composition into a target site, localization of the polymeric nanowires after ingesting, nasal inhalation, or intravenous delivery, or through release of the polymeric nanowires from an implanted device at the target site. Polymeric nanowires and methods for preparing compositions comprising polymeric nanowires are described, for example, in US20200023068.

The amount of bioactive compound will depend on the site of application, the condition being treated and the type of bioactivity desired. In some embodiments, individual polymeric nanowires may include 0.001 ng or greater of the bioactive agent, such as 0.01 ng or greater, 0.0001 μg or greater of the bioactive compound, such as 0.001 μg or greater, such as 0.01 μg or greater, such as 0.1 μg or greater, such as 1 μg or greater, such as 10 μg or greater, such as 25 μg or greater, such as 50 μg or greater, such as 100 μg or greater such as 500 μg or greater, such as 1000 μg or greater such as 5000 μg or greater and including 10,000 μg or greater. Where the bioactive compound is incorporated into the polymeric nanowires as a liquid, the concentration of bioactive compound may be 0.0001 ng/mL or greater, such as 0.001 ng/mL or greater, such as 0.01 ng/mL or greater, such as 0.1 ng/mL or greater, such as 0.5 ng/mL or greater, such as 1 ng/mL or greater, such as 2 ng/mL or greater, such as 5 ng/mL or greater, such as 10 ng/mL or greater, such as 25 ng/mL or greater, such as 50 ng/mL or greater, such as 100 ng/mL or greater such as 500 ng/mL or greater, such as 1000 ng/mL or greater such as 5000 ng/mL or greater and including 10,000 ng/mL or greater.

Depending on the amount of bioactive compound associated with the individual polymeric nanowires, compositions of individual polymeric nanowires have a concentration of bioactive compound that is 0.001 nM or greater, such as 0.005 nM or greater, such as 0.01 nM or greater, such as 0.05 nM or greater, such as 0.1 nM or greater, such as 0.5 nM or greater, such as 1 nM or greater, such as 5 nM or greater, such as 10 nM or greater, such as 50 nM or greater, such as 100 nM or greater, such as 250 nM or greater and including 500 nM or greater.

In certain embodiments, the polymeric nanowires are formulated to release the at least one bioactive compound at a target site. In one embodiment, the at least one bioactive compound is released from the within the pores of each individual polymeric nanowire in the subject compositions. In another embodiment, the at least one bioactive compound is released by cleavage of a linker between the polymeric nanowire and the bioactive compound. For example, the linker may be enzymatically cleaved or cleaved by hydrolysis. Where the linker is enzymatically cleaved, linkers of interest may include enzymatically cleavable moiety.

Release of the bioactive compound by the polymeric nanowires may be a sustained release or pulsatile release. By “sustained release” is meant that the bioactive compound is associated with the polymeric nanowires to provide for constant and continuous delivery of at least one bioactive compound over the entire time the polymeric nanowires are maintained in contact with the site of administration, such as over the course of 1 minute or longer, such as 5 minutes or longer, such as 10 minutes or longer, such as 15 minutes or longer, such as 30 minutes or longer, such as 45 minutes or longer, such as 1 hour or longer, such as 6 hours or longer, such as 12 hours or longer, such as 1 day or longer, such as 2 days or longer, such as 5 days or longer, such as 10 days or longer, such as 15 days or longer, such as 30 days or longer and including 100 days or longer. For example, the bioactive compound may be associated with the polymeric nanowires to provide for constant and continuous delivery over that ranges, such as from 1 day to 30 days, such as from 2 days to 28 days, such as from 3 days to 21 days, such as from 4 days to 14 days and including from 5 days to 10 days.

In other embodiments, the individual polymeric nanowires are configured to provide a pulsatile release of the at least one bioactive compound. By “pulsatile release” is meant that the polymeric nanowires release the at least one bioactive compound into the site of administration incrementally (e.g., at discrete times), such as every 1 minute, such as every 5 minutes, such as every 10 minutes, such as every 15 minutes, such as every 30 minutes, such as every 45 minutes, 1 hour, such as every 2 hours, such as every 5 hours, such as every 12 hours, such as every 24 hours, such as every 36 hours, such as every 48 hours, such as every 72 hours, such as every 96 hours, such as every 120 hours, such as every 144 hours and including every 168 hours or some other increment.

In other embodiments, the subject polymeric nanowires are degradable over time and deliver the at least one bioactive compound after a certain amount of the polymeric nanowire has degraded. For example, an amount of the at least one bioactive compound may be delivered after every 10% of the polymeric nanowire has degraded, such as after every 15% of the polymeric nanowire has degraded, such as after every 20% of the polymeric nanowire has degraded, such as after every 25% of the polymeric nanowire has degraded, such as after every 30% of the polymeric nanowire has degraded and including after every 33% of the polymeric nanowire has degraded at the site of administration.

In still other embodiments, individual polymeric nanowires employed in the present disclosure release a large amount of the at least one bioactive compound immediately upon administration at the target site, such as for example 50% or more, such as 60% or more, such as 70% or more and including 90% or more of the at least one bioactive compound are released immediately upon administration. In yet other embodiments, the individual polymeric nanowires release the at least one bioactive compound at a predetermined rate, such as at a substantially zero-order release rate, such as at a substantially first-order release rate or at a substantially second-order release rate.

In certain embodiments, the individual polymeric nanowires may have diameters that range from 10 nm to 500 nm, such as from 15 nm to 400 nm, such as from 20 nm to 300 nm, such as from 25 nm to 200 nm and including from 50 nm to 100 nm, such as a 200 nm diameter and have a length that is 0.01 μm or more, such as 0.05 μm or more, such as 0.1 μm or more, such as 0.5 μm or more, such as 1 μm or more, such as 2 μm or more, such as 3 μm or more, such as 5 μm or more, such as 10 μm or more, such as 15 μm or more, such as 20 μm or more, such as 25 μm or more, such as 30 μm or more, such as 50 μm or more, such as 100 μm or more, such as 150 μm or more, such as 200 μm or more and including 250 μm or more or more. In additional embodiments, individual polymeric nanowires having at least one bioactive compound have a length of from about 10 μm to about 20 μm and a diameter of from about 10 nm to about 500 nm.

### Microrods

In certain embodiments of the methods, uses, and compositions of the disclosure, the biomaterial carriers comprise microrods. In further embodiments, the microrods include at least one bioactive compound, for example, painless NGF. Microrods can have any three-dimensional shape. In some embodiments, microrods have a three-dimensional shape of any regular polyhedron, any irregular polyhedron, and combinations thereof. The shape of a microrod is, in some embodiments, dictated by specific tissues, specific locations in specific tissues, or specific modes of administration or implantation. For example, an injectable scaffold may require microrods of a shape that is amenable to the flow in an injection stream.

In certain embodiments, microrods having an increased surface area are beneficial. Surface area of any microrod may be increased, for example, by synthesizing the microrod having a textured surface and/or, for example, to be porous. Microrods and their preparation are described, for example, in U.S. Pat. No. 8,591,933.

In certain embodiments, the microrods employed herein as biomaterial carriers are synthesized from one or more polymers, one or more copolymers, one or more block polymers (including di-block polymers, tri-block polymers, and/or higher multi-block polymers), as well as combinations thereof. Useful polymers include, but are not limited to, polylactic acid (PLA), polyglycolic acid (PGA), poly(.epsilon.-caprolactone) (PCL), poly(ethylene glycol) diacrylate (PEGDA), poly(ethylene glycol) dimethacrylate (PEGDMA), SU-8, poly(methyl methacrylate), poly(lactide-co-glycolide), poly-caprolactone, and elatin/caprolactone, collagen-GAG, collagen, fibrin, poly(anhydrides), poly(hydroxy acids), poly(ortho esters), poly(propylfumerates), polyamides, polyamino acids, polyacetals, biodegradable polycyanoacrylates, biodegradable polyurethanes and polysaccharides, polypyrrole, polyanilines, polythiophene, polystyrene, polyesters, non-biodegradable polyurethanes, polyureas, poly(ethylene vinyl acetate), polypropylene, polyethylene, polycarbonates, poly(ethylene oxide), polydioxanone, “pseudo-polyamino acid” polymer based on tyrosine, tyrosine-derived polycarbonate poly(DTE-co-DT carbonate), tyrosine-derived polyarylate, polyanhydride, trimethylene carbonate, poly(.beta.-hydroxybutyrate), poly(g-ethyl glutamate), poly(DTH iminocarbonate), poly(bisphenol A iminocarbonate), poly(ortho ester), polycyanoacrylate, and polyphosphazene, poly(lactide-co-glycolide) (PLGA), poly(DL-lactide-co-.epsilon.-caprolactone) (DLPLCL), a modified polysaccharide (cellulose, chitin, dextran) a modified protein, casein- and soy-based biodegradable thermoplastics, collagen, polyhydroxybutyrate (PHB), multiblock copolymers of poly(ethylene oxide) (PEO) and poly(butylene terephthalate) (PBT), polyrotaxanes. In other embodiments, the microrods are formed from one or more phospholipids. 2-methacryloyloxyethyl phosphorylcholine (MPC), one or more cationic polymers (poly(a-[4-aminobutyl]-L-glycolic acid), or one or more silicone-urethane copolymers. In still other embodiments, microrods are formed from co-polymers of any of the above, mixtures of the above, and/or adducts of the above. The ordinarily skilled artisan will readily appreciate that any other known polymer is suitable for making microrods of the instant scaffolds. In certain embodiments, the PEG composition is significant to controlling drug release by controlling the pore size.

By employing photolithography, PEGDMA microrods can be produced in a high throughput fashion. Moreover, β-NGF loading onto PEGDMA microrods can be increased using 90% (v/v) PEGDMA macromer. Tuning the cross-linking densities can alter the polymer mesh size within the hydrogel and subsequent drug loading and release (Hoare and Kohane, 2008, Polymer 49:1993-2007; Li and Mooney, 2016, Nature Reviews Materials 1:1-17). Higher concentrations of PEG have shown greater loading capacities (Stukel, et al., 2015, J Biomed Materials Res Part A 103:604-613). Striking visual differences in loading are apparent between lower and higher molecular weight molecules such as DAPI and FITC-BSA. DAPI stains the PEGDMA microrods uniformly, versus the FITC-BSA that can mostly be visualized on the surface of the PEGDMA microrods immediately after loading. Given that DAPI has a molecular weight of 0.277 kDa, compared to the 67 kDa size of FITC-BSA, micrographs confirmed that molecules of smaller size can more readily diffuse across or into the PEGDMA polymer mesh network (data not shown).

In order to demonstrate that the loaded β-NGF retained its bioactivity, an in vitro proliferation assay was performed using the TrkA expressing TF-1 cell line (Ma and Zou, 2013, J Applied Virology 2(2):32). The assay was performed with 16,000 PEGDMA microrods, versus 100,000 used for the loading assay, as only 16,000 could effectively be aspirated in a 20 μL syringe used for the in vivo experiments. Approximately 30-40% of total protein loaded in 100,000 microrods was calculated to be about 1-2 mg. Therefore, the highest calculation of 2 μg (2000 ng) was loaded into the 16,000 microrods and was set as the soluble NGF amount for all experiments in parallel. However, the highly specific ELISA assay measured only ˜18 ng of β-NGF in 16,000 PEGDMA microrods, suggesting that some of the β-NGF proteins may lose their native molecular arrangement during loading or elution, thus reducing bioactivity. Presumably, the discrepancy between the microBCA assay and the ELISA calculations may be attributed to the disruption of β-NGF's non-covalent homodimer confirmation or the non-specificity of the microBCA assay. Nonetheless, 16,000 PEGDMA microrods containing 18 ng of bioactive β-NGF's had a potent effect on TF-1 cell proliferation, likely driven by the sustained release of β-NGF over the 96-hour experimental period. A nominal increase in proliferation of cells cultured with non-loaded PEGDMA microrods was also observed. Although the degradation products of the PEGDMA microrods were not evaluated herein, PEG at low concentrations have previously been shown to slightly elevate cell proliferation, which could be contributing to TF-1 cell proliferation (Bahney, et al., 2014, J Bone Mineral Res 29(5)).

In specific embodiments, microrods are, on average, each about 0.01, about 0.05, about 0.1, about 0.5, about 1, about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, about 400, about 405, about 410, about 415, about 420, about 425, about 430, about 435, about 440, about 445, about 450, about 455, about 460, about 465, about 470, about 475, about 480, about 485, about 490, about 495, about 500, about 505, about 510, about 515, about 520, about 525, about 530, about 535, about 540, about 545, about 550, about 555, about 560, about 565, about 570, about 575, about 580, about 585, about 590, about 595, about 600, about 605, about 610, about 615, about 620, about 625, about 630, about 635, about 640, about 645, about 650, about 655, about 660, about 665, about 670, about 675, about 680, about 685, about 690, about 695, about 700, about 705, about 710, about 715, about 720, about 725, about 730, about 735, about 740, about 745, about 750, about 755, about 760, about 765, about 770, about 775, about 780, about 785, about 790, about 795, about 800, about 805, about 810, about 815, about 820, about 825, about 830, about 835, about 840, about 845, about 850, about 855, about 860, about 865, about 870, about 875, about 880, about 885, about 890, about 895, about 900, about 905, about 910, about 915, about 920, about 925, about 930, about 935, about 940, about 945, about 950, about 955, about 960, about 965, about 970, about 975, about 980, about 985, about 990, about 995, or about 1000 or more microns in length.

In additional embodiments, microrods have a cross-sectional area of about A microns times B microns, wherein A and B are independently selected from about 1, about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, about 400, about 405, about 410, about 415, about 420, about 425, about 430, about 435, about 440, about 445, about 450, about 455, about 460, about 465, about 470, about 475, about 480, about 485, about 490, about 495, about 500, about 505, about 510, about 515, about 520, about 525, about 530, about 535, about 540, about 545, about 550, about 555, about 560, about 565, about 570, about 575, about 580, about 585, about 590, about 595, about 600, about 605, about 610, about 615, about 620, about 625, about 630, about 635, about 640, about 645, about 650, about 655, about 660, about 665, about 670, about 675, about 680, about 685, about 690, about 695, about 700, about 705, about 710, about 715, about 720, about 725, about 730, about 735, about 740, about 745, about 750, about 755, about 760, about 765, about 770, about 775, about 780, about 785, about 790, about 795, about 800, about 805, about 810, about 815, about 820, about 825, about 830, about 835, about 840, about 845, about 850, about 855, about 860, about 865, about 870, about 875, about 880, about 885, about 890, about 895, about 900, about 905, about 910, about 915, about 920, about 925, about 930, about 935, about 940, about 945, about 950, about 955, about 960, about 965, about 970, about 975, about 980, about 985, about 990, about 995, or about 1000 or more microns.

In specific embodiments, at least one bioactive compound, for example, painless NGF is associated with the microrods by covalent interaction. In other specific embodiments, the at least one bioactive compound, for example, painless NGF, is associated with the microrods by non-covalent association. In a covalent interaction, the at least one bioactive compound is directly attached to the microrod through any suitable means. Alternatively, the at least one bioactive compound is attached to the microrod through a space or linker that has no biological activity itself, or through a second bioactive compound that possesses the same or a different biological activity compared to the first bioactive compound. In still another embodiment, the at least one bioactive compound is elutable from the microrod. Elutable means that the at least one bioactive compound can be separated from the microrods through, for example, simply diffusion, cleavage of a covalent bond, dissociation or some other type of interaction. The at least one bioactive compound may, in some embodiments, be released in a controlled manner and, in other embodiments, the release is bolus in nature.

In yet another embodiment, the biomaterial carriers are associated with a targeting molecule that interacts with a target cell or tissue expressing a binding partner for said targeting molecule. In specific embodiments, the targeting molecule is selected from, without limitation, a cell adhesion molecule, a cell adhesion molecule ligand, an antibody immunospecific for an epitope expressed on the surface of a target cell type, and any member of a binding pair, wherein one member of the binding pair is expressed on the target cell or tissue of interest.

### Administration

One aspect of the present disclosure includes administering a pharmaceutical composition comprising at least one bioactive compound, for example, painless nerve growth factor (NGF) to a subject. Further aspects of the present disclosure include administering a pharmaceutical composition comprising biomaterial carriers comprising at least one bioactive compound, for example, painless nerve growth factor (NGF) to a subject. In practicing the methods and uses according to certain embodiments of the disclosure, a composition of a plurality of individual nanowires, microrods, or other biomaterial carriers having a bioactive compound, for example, painless NGF, is administered to a subject.

In certain embodiments, a pharmaceutical composition comprising biomaterial carriers comprising at least one bioactive compound are administered locally. The terms “local” and “locally”, as used herein, refer to in the fracture gap, adjacent to the fracture site, adjacent to the fracture callus, along the periosteum, and/or within the intramedullary canal. In further embodiments, the composition may be administered to a tissue of a subject, at, next to, or near the fracture callus.

Any convenient mode of administration may be employed. Modes of administration may include, but are not limited to injection (e.g., percutaneously, subcutaneously, intravenously, or intramuscularly, intrathecally). The composition can be administered alone or applied to a bone graft or scaffold, for example, a sponge, for example, a collagen sponge. In certain embodiments, the composition further comprises an agent that targets the biomaterial carrier comprising at least one bioactive compound (for example, the NGF-eluting microrod) to a fracture site. In further embodiments, the agent is a bone autograft, an allograft, or an antibiotic cement bead.

In certain embodiments, the individual biomaterial carriers localize at the target location over a predetermined period of time. The term “localizes” is used herein in its conventional sense to refer to concentrating or accumulating administered individual nanowires or microrods, for example, within a predetermined area of the target site, such as within an area of 50 mm² or less, such as 40 mm² or less, such as 30 mm² or less, such as 25 mm² or less, such as 20 mm² or less, such as 15 mm² or less, such as 10 mm² or less, such as 9 mm² or less, such as 8 mm² or less, such as 7 mm² or less, such as 6 mm² or less, such as 5 mm² or less, such as 4 mm² or less, such as 3 mm² or less, such as 2 mm² or less, such as 1 mm² or less, such as 0.5 mm² or less, such as 0.1 mm² or less, such as 0.05 mm² or less and including a predetermined area of 0.001 mm² or less. In some instances, 10% or more of the administered individual nanowires or microrods in the composition localizes within an area of the target site, such as 25% or more, such as 50% or more, such as 55% or more, such as 60% or more, such as 65% or more, such as 70% or more, such as such as 75% or more, such as 80% or more, such as 85% or more, such as 90% or more, such as 95% or more, such as 96% or more, such as 97% or more, such as 98% or more, such as 99% or more and including 99.9% or more of the administered individual nanowires or microrods in the composition localizes within an area of the target site, such as within an area of 50 mm² or less, such as 40 mm² or less, such as 30 mm² or less, such as 25 mm² or less, such as 20 mm² or less, such as 15 mm² or less, such as 10 mm² or less, such as 9 mm² or less, such as 8 mm² or less, such as 7 mm² or less, such as 6 mm² or less, such as 5 mm² or less, such as 4 mm² or less, such as 3 mm² or less, such as 2 mm² or less, such as 1 mm² or less, such as 0.5 mm² or less, such as 0.1 mm² or less, such as 0.05 mm² or less and including a predetermined area of 0.001 mm² or less.

### Pharmaceutical Compositions

The disclosure provides pharmaceutical compositions comprising i) nerve growth factor (NGF) and ii) a pharmaceutically acceptable carrier for use in stimulating bone healing in a subject, accelerating bone healing in a subject, and/or improving bone healing in a subject. The disclosure also provides pharmaceutical compositions comprising i) biomaterial carriers comprising nerve growth factor (NGF) and ii) a pharmaceutically acceptable carrier for use in stimulating bone healing in a subject, accelerating bone healing in a subject, and/or improving bone healing in a subject. The disclosure also provides pharmaceutical compositions comprising i) nerve growth factor (NGF) and ii) a pharmaceutically acceptable carrier for use in treating bone fracture in a subject. The disclosure also provides pharmaceutical compositions comprising i) biomaterial carriers comprising nerve growth factor (NGF) and ii) a pharmaceutically acceptable carrier for use in treating bone fracture in a subject. Pharmaceutical compositions in accordance with the disclosure are administered with suitable excipients, and/or other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell, et al., “Compendium of excipients for parenteral formulations”, PDA (1998), J Pharm Sci Technol 52:238-311.

In certain embodiments, the excipient is simply water, and in one embodiment, pharmaceutical grade water. In other embodiments, the excipient is a buffer, and in one embodiment, the buffer is pharmaceutically acceptable. Buffers may also include, without limitation, saline, glycine, histidine, glutamate, succinate, phosphate, acetate, aspartate, or combinations of any two or more buffers.

In other embodiments, a matrix is included in the composition. In additional embodiments, the matrix is viscous, yet still flowable, and in other embodiments, the matrix is solid, semi-solid, gelatinous or of any density in between. Accordingly, in various embodiments and without limitation, the matrix is collagen, gelatin, gluten, elastin, albumin, chitin, hyaluronic acid, cellulose, dextran, pectin, heparin, agarose, fibrin, alginate, carboxymethylcellulose, Matrige™ (a hydrogel formed by a solubilized basement membrane preparation extracted from the Engelbreth-Holm-Swarm (EHS) mouse sarcoma), hydrogel organogel, or mixtures and/or combinations thereof. Again, the worker of ordinary skill in the art will appreciate that any pharmaceutical grade matrix is amenable for use in a composition of the disclosure.

In certain embodiments, the dose of biomaterial carriers comprising at least one bioactive compound is fixed (i.e., is not based on body weight of the subject to which it is administered). In additional embodiments, the dose is about 1 ng/day to about 500 ng/day, about 5 ng/day to about 250 ng/day, about 10 ng/day to about 100 ng/day, or about 25 ng/day to about 50 ng/day. In certain embodiments, the release of the at least one bioactive compound from the biomaterial carrier is a non-linear burst release. In other embodiments, the dose of biomaterial carriers comprising at least one bioactive compound may vary depending upon the age and the size of a subject to be administered, the type/severity of fracture, the location of fracture, conditions, route of administration, and the like. When the biomaterial carriers comprising at least one bioactive compound disclosed herein are used for treating a bone fracture in a patient, it is advantageous to administer the biomaterial carriers comprising at least one bioactive compound normally at a single dose of about 0.1 to about 100 mg/kg body weight. In specific embodiments, the dose/dosage is based on average release of bioactive compound from carrier at the site of administration/the target site.

In certain embodiments, the frequency and the duration of the treatment (administration) can be adjusted. In certain embodiments, the biomaterial carriers comprising at least one bioactive compound disclosed herein can be administered as an initial dose, followed by administration of a second or a plurality of subsequent doses of the biomaterial carriers comprising at least one bioactive compound in an amount that can be approximately the same or less than that of the initial dose, wherein the subsequent doses are separated by at least one week, at least 2 weeks; at least 3 weeks; at least one month; or longer, based on a lack of adequate progression of healing parameters. In certain embodiments, a lack of adequate progression of healing parameters comprises no mineralization on X-ray, low mineralization on X-ray, no reduction in pain, minimal reduction in pain, no increase in stability, and/or minimal increase in stability. A clinician would be able to change the frequency and duration of treatment on a per patient basis based on their diagnosis and unique condition.

In certain embodiments, the pharmaceutical composition can be delivered in a controlled release system. In one embodiment, a pump may be used. In another embodiment, polymeric materials can be used. In yet another embodiment, a controlled release system can be placed in proximity of the composition's target, thus requiring only a fraction of the systemic dose.

The injectable preparations may include dosage forms for intravenous, subcutaneous, percutaneous, intramuscular injections, drip infusions, etc. These injectable preparations may be prepared by methods publicly known.

A pharmaceutical composition of the present disclosure can, in certain embodiments, be delivered subcutaneously or percutaneously with a standard needle and syringe. In addition, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered, and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Pharmaceutical compositions according to the disclosure are, in specific embodiments, for use in stimulating bone fracture healing, for use in accelerating bone fracture healing, for improving bone fracture healing, and for use in treating bone fracture in a subject.

### Therapeutic Uses

The bioactive compound comprised in biomaterial carriers of the present disclosure, for example, painless NGF in a microrod, is, in specific embodiments, useful for the treatment of bone fracture, for the stimulation of bone fracture healing, for the acceleration of bone fracture healing, and for the improvement of bone fracture healing in a subject in need thereof. The bioactive compound itself, for example, NGF or painless NGF, is, in specific embodiments, useful for the treatment of bone fracture, for the stimulation of bone fracture healing, for the acceleration of bone fracture healing, and for the improvement of bone fracture healing in a subject in need thereof.

In additional embodiments of the disclosure, the bioactive compound comprised in biomaterial carriers is used for the preparation of a pharmaceutical composition or medicament for treating bone fracture in a patient, stimulating bone fracture healing, accelerating bone fracture healing, improving bone fracture healing. In still another embodiment of the disclosure, the bioactive compound comprised in biomaterial carriers is used as adjunct therapy with any other agent or any other therapy known to those skilled in the art useful for treating bone fracture.

### Combination Therapies

Combination therapies may include a bioactive compound comprised in a biomaterial carrier and any additional therapeutic agent that may be advantageously combined with the compound and carrier. The bioactive compound comprised in a biomaterial carrier may be combined synergistically with one or more drugs or therapy used to treat bone fracture and/or a symptom associated with bone fracture.

In some embodiments, the bioactive compound comprised in a biomaterial carrier may be used in combination with one or more additional therapeutic agents/therapies including, but not limited to, protein supplements (e.g., including lysine, arginine, proline, glycine, cysteine, glutamine), antioxidants (e.g., vitamin E, vitamin C, lycopene, alpha-lipoic acid), mineral supplements (e.g., calcium, iron, potassium, zinc, copper, phosphorus, bioactive silicon), vitamin supplements (e.g., B (B6), C, D, and/or K), herbal supplements (e.g., comfrey, arnica, horsetail grass, Cissus quadrangularis), anti-inflammatory nutrients (e.g., quercetin, flavonoids, omega-3 fatty acids, proteolytic enzymes), and exercise. In still other embodiments, the bioactive compound comprised in a biomaterial carrier may be sequentially dosed with another drug, for example, a pro-chondrogenic (e.g., TGFb or maybe even PTH/PTHrP) prior to mutant NGF, causing conversion of cartilage to bone

As used herein, the term “in combination with” means that at least one additional therapeutic agent/therapy may be administered prior to, concurrent with, or after the administration of the bioactive compound comprised in a biomaterial carrier. The term “in combination with” also includes sequential or concomitant administration of a bioactive compound comprised in a biomaterial carrier and at least one additional therapeutic agent/therapy.

“Concurrent” administration, for purposes of the present disclosure, includes, e.g., administration of a bioactive compound comprised in a biomaterial carrier and at least one additional therapeutic agent/therapy to a subject in a single dosage form, or in separate dosage forms administered to the subject within about 30 minutes or less of each other. If administered in separate dosage forms, each dosage form may be administered via the same route (e.g., both the bioactive compound comprised in a biomaterial carrier and the at least one additional therapeutic agent/therapy may be administered percutaneously, etc.); alternatively, each dosage form may be administered via a different route (e.g., the bioactive compound comprised in a biomaterial carrier may be administered percutaneously, and the at least one additional therapeutically active component may be administered orally). In any event, administering the components in a single dosage from, in separate dosage forms by the same route, or in separate dosage forms by different routes are all considered “concurrent administration,” for purposes of the present disclosure. For purposes of the present disclosure, administration of a bioactive compound comprised in a biomaterial carrier “prior to”, “concurrent with,” or “after” (as those terms are defined herein above) administration of at least one additional therapeutic agent/therapy is considered administration of a bioactive compound comprised in a biomaterial carrier “in combination with” at least one additional therapeutic agent/therapy.

### Kits

In an additional aspect, the disclosure provides kits, wherein the kits include at least one or more, e.g., a plurality of, the components needed to prepare a composition of biomaterial carriers comprising at least one bioactive compound disclosed herein. In certain embodiments, one or more of each component may be provided as a packaged kit, such as in individual containers (e.g., pouches). Kits may further include other components for practicing the subject methods, such as measuring and application devices (e.g., syringes), as well as containers for solutions such as beakers and volumetric flasks.

In addition, kits may include step-by-step instructions for how to practice the subject methods. As such, the instructions may be present in the kits as a package insert, in the labeling of the container of the kit or components thereof (i.e., associated with the packaging or subpackaging), etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided.

### EXAMPLES

The following examples are put forth so as to provide those of ordinary skill in the art with a complete disclosure and description of how to make and use the methods and compositions of the disclosure, and are not intended to limit the scope of what the inventors regard as their disclosure. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, room temperature is about 25° C., and pressure is at or near atmospheric.

### Example 1: Painless NGF

1.1 Painless NGF does not Induce Thermal Hyperalgesia

In order to characterize pain sensation associated with NGF^{WT}-nanowires versus NGF^{R100W}-nanowires, hyperalgesia first needed to be assessed for NGF^{R100W}. Intradermal delivery of 200 ng of soluble NGF^{R100W} did not induce acute mechanical or thermal hyperalgesia, per the Randal-Selitto and Hargreaves test, respectively (Sung, et al., 2018, J Neurosci 38:3394-3413). New dose escalation testing demonstrates that NGF^{R100W} does not induce pain sensitization at a dose 10-fold higher than WT NGF (FIGS. 2A and 2B). Specifically, 0.5 mg of WT NGF caused significant thermal pain at 20 and 45 min after injection, while NGF^{R100W} did not at 5.0 mg (FIG. 2A). The reduction in nociceptive threshold induced by painless NGF suggests a dose-dependent effect, although preliminary testing did not reach statistical significance (FIG. 2B). The nociceptive threshold of the NGF^{WT}- and NGF^{R100W}-nanowires is also tested by performing intradermal injections of 200 mg of nanowires loaded with 0.5-5 mg of protein. Thermal and mechanical hyperalgesia are measured using the Hargreaves technique and Randal-Selitto threshold test as previously. In clinical trials with NGF, pain sensation was associated with a quick onset (Sung, et al., 2019, Neural Regen Res 14:570-573); consequently testing is at 20- and 45-minutes post-injection as shown in FIG. 2B. Statistically significant reductions in pain sensitization with NGF^{R100W} compared to WT NGF are a primary success outcome. Based on the mean and standard deviation of the data set shown in FIGS. 2A and 2B, a power analysis indicates 6 mice/group are required to achieve a power level >80% with an effect size d=1.5 and a significance level of 5%.

### 1.2 NGF^{R100W} Promotes Regeneration of Paw Skin Sensory Nerves in CMT2B Mutant Mice

The trophic functionality of the NGF^{R100W}-nanowires is validated in vivo by quantifying regeneration of the intraepidermal nerve fibers (IENFs) and subepidermal neural plexus (SNP) in the Charcot-Marie-Tooth 2B (CMT2B) murine model of peripheral sensory neuropathy. CMT2B is caused by missense mutations in Rab7 GTPase that leads to an axonal length-dependent degeneration of sensory fibers. The CMT2B knockin mouse model was generated, in which the mutant mice develop significant peripheral sensory deficits at 9 months of age. This genetic model is an effective tool for quantifying neuronal regeneration.

NGF^{R100W} was confirmed to be as effective as NGF in promoting neuronal regeneration. (FIGS. 3A-3F). Similarly, the dose-dependent regenerative capacity of NGF^{WT}- and NGF^{R100W}-nanowires are tested in the CMT2B mouse by giving 2 therapeutic injections 3 weeks apart, then measuring IENF and SNP density at 6 weeks following injection using quantitative immunohistochemistry to the pan neuronal PGP9.5 marker (Yang, et al., 2020, Prog Neurobiol 194: 101866). NGF/NGF^{R100W} nanowires are compared to soluble NGF/NGF^{R100W}, empty nanowires, and placebo injections. Using the mean and standard deviation from the PGP9.5 density data shown in FIG. 3D, quantified by ImageJ, a power analysis was conducted using G*Power to determine that 3 mice/group are required to achieve a power level >80% with an effect size d=1.5 and a significance level of 5%. Successful trophic functionality is judged by a statistically significant increase in neuronal density with NGF/NGF^{R100W} nanowires relative to both soluble protein delivery and negative controls (PBS, empty nanowires).

1.3 Validation that NGF^{R100W} Activates Osteogenesis in Chondrogenic Cells

To date, the trophic activity of NGF^{R100W} has only been validated in neuronal tissues (FIGS. 3A-3F). An in vitro dose from 0.02-20,000 ng/mL was tested on the chondrogenic cell line (ATDC5), and increased osteocalcin expression was found with higher doses of NGF (data not shown). The NGF^{R100W} mutant has been shown to be capable of binding to the TrkA receptor and activating downstream signaling pathways in neuronal cells (Sung, et al., 2018, J Neurosci 38:3394-3413). Based on the dose response curve generated by treating cultured and matured ATDC5 cells with increasing doses of NGF^{wt} for 24 hours and measuring the osteogenic changes via the canonical osteoblast gene osteocalcin, as mentioned above, a dose of 20 μg/mL NGF or NGF^{R100W} was used for treatment of the ATDC5s and to then look for downstream activation using cFos. NGF^{R100W} was found to activate TrkA signaling in chondrocytes using cFOS expression as a downstream marker of TrkA activation (data not shown).

In order to confirm that painless NGF had similar trophic capabilities compared to WT NGF in the target cell population of chondrocytes, the well-established ATDC5 chondrogenic cell line was utilized and treated with 0.2 or 20 μg of NGF or NGF^{R100W} compared to negative control (FIGS. 3G-3L). Data show that Vegf is significantly upregulated by 24 hours of NGF and NGF^{R100W} treatment (FIG. 3G), but that Axin2 was not (data not shown). Wnt pathway activation is more rigorously tested by transfecting either the Wnt responsive TOPFlash (M50 Super 8×TOPFlash, Addgene #12456), or the mutant FOPFlash plasmid vector, (M51 Super 8×FOPFlash, Addgene, #12457), into ATDC5 cells using SuperFect Transfection Reagent. Transfection is confirmed by co-transfecting the constitutively activated Renilla luciferase (pLX313-Renilla luciferase, Addgene #118016). Wnt response to NGF and NGF^{R100W} is quantified on a fluorescent/luciferase plate reader.

Next, qRT-PCR (lhh, gli1, ptch1) and the Gli-Reporter (7Gli::GFP Addgene #110494) are used, similar to above, to measure NGF-mediated activation of the lhh pathway in the ATDC5 chondrocytes. The data suggest a strong activation of Ihh by the painless NGF mutant 1 hour following treatment (FIG. 3I), indicating that it may play an early role in stimulating downstream EO, which can be further confirmed by the Gli-Reporter assay and temporal testing of candidate pathways. NGF^{R100W} (green) showed enhanced bioactivity relative to NGF^{WT} (blue) at a concentration of 0.2 mg through upregulated endochondral (Indian hedgehog, lhh) and osteogenic (alkaline phosphatase and osteocalcin) genes (FIGS. 3J-3L). In vitro, a higher [NGF] was not found to be more osteogenic. These data support that NGF^{R100W} should provide, at minimum, a trophic equivalence compared to NGF in the fracture model used herein.

To maximize the osteogenic dose of NGF^{R100W} while minimizing nociception upon injection, 0.5 μg NGF^{R100W}/day delivered during the endochondral phase of repair (days 7-9), is compared to 5 and 50 μg NGF^{R100W}/day. Pain sensation is tested by thermal (hot plate, cold acetone) and mechanical (electronic Von Frays) allodynia tests following NGF or NGF^{R100W} injection. TrkA pathway activation is measured at day 10, 24 hours following the last NGF dosing, by cFOS qRT-PCR.

### Pain Sensation

In clinical trials with NGF, pain sensation was associated with a quick onset (Sung, et al., 2019, Neural Regen Res 14:570-573). Consequently, nociception is evaluated 30, 60, and 90 minutes following injections using the standardized assays for thermal and mechanical allodynia (Deius, et al., 2014, Neuro Oncol 16:1324-1332). Hot plate technique is used as previously employed for testing nociception of NGF^{R100W} compared to NGF (Sung, et al., 2018, J Neurosci 38:3394-3413; Yang, et al., 2019, bioRxiv 784660), as well as cold test by acetone on hind paw (Choi, et al., 1994, Pain 59:369-376). Aversion to mechanical stimuli is measured using the electronic Von Frey method (Deius, et al., 2014, Neuro Oncol 16:1324-1332).

### Fracture Healing

Functional fracture healing outcomes are measured (histomorphometry, quantitative μCT, biomechanics) at only 14- and 28-days post-injury in effort to capture the most critical healing points and minimize overall animal number. In addition to quantifying bone healing at different doses, spleen, liver, and blood are harvested at euthanasia to check for any negative systemic inflammatory effect (Morioka, et al., 2019, Sci Reports 9:12199).

### Example 2: Local Injections of β-NGF Accelerate Endochondral Fracture Repair by Promoting Cartilage to Bone Conversion

### Tibia Fracture Model

Briefly, adult (10-16 weeks) male mice were anesthetized via inhalant isoflurane, and closed non-stable fractures were made mid-diaphysis of the tibia via three-point bending fracture device (Bahney, et al., 2014, J Bone Miner Res 29:1269-1282). Fractures were not stabilized, as this method promotes robust endochondral repair. After fractures are created, animals were provided with post-operative analgesics (buprenorphine sustained-release). Animals were socially housed and allowed to ambulate freely.

### Mice

Studies involving wildtype mice were conducted on the C57BL/6J strain obtained from Jackson Labs (Stock #000664). NGF-eGFP express eGFP under the control of the mouse NGF promoter (Kawaja, et al., 2011, J Comp Neurol 519:2522-2545). TrkA-LacZ mice have a LacZ sequence inserted immediately following the ATG in exon 1 of the mouse Ntrk1 gene (Moqrich, et al., 2004, Nat Neurosci 7:812-818). Axin2-eGFP mice express eGFP under the Axin2 promoter/intron 1 sequences (Jho, et al., 2002, Mol Cell Biol 22:1172-1183). β-NGF and control injections

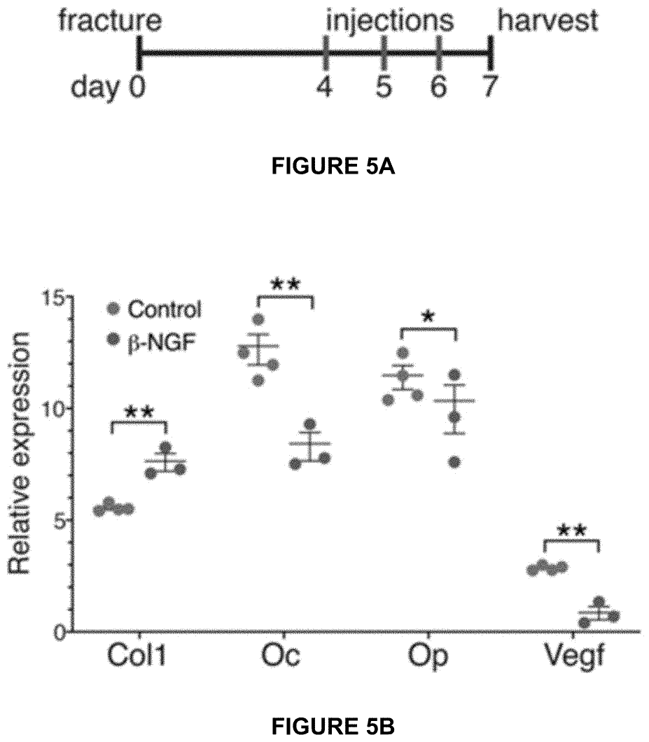

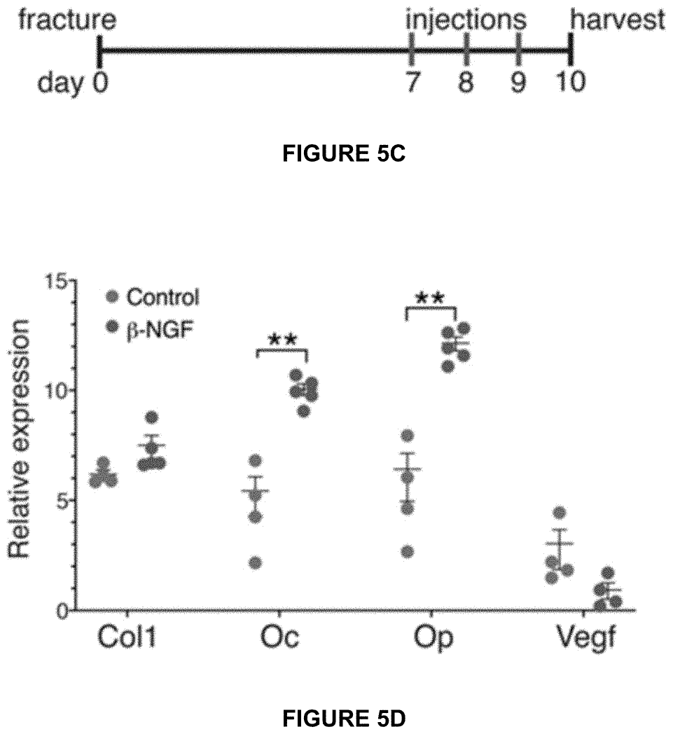

Two time points were initially tested to compare osteogenic marker expression within fracture calluses. Injections were administered once daily for 3 days beginning either four days or seven days post-fracture (FIG. 5C, 5D). Experimental groups consisted of 0.5 μg of recombinant human β-NGF in 20 μL of basal media versus control injections of basal media-only (DMEM basal media, Gibco cat #A1443001) using a Hamilton syringe guided by fluoroscopy. A dosage of 0.5 μg/day was derived from earlier protocols, wherein the dose herein lies between 0.1-1.4 μg/day (Grills, et al., 1997, J Orthoped Res 15:235-242; Shinoda, et al., 2011, J Neurosci 31:7145-7155). mRNA isolation and RT-qPCR

After β-NGF administration, calluses were harvested 24 h following the final injection. After callus dissections, tissue samples were homogenized in Trizol, then mRNA was extracted from tissue lysates by use of RNeasy Mini Kit following the manufacturer's instructions (Qiagen cat #74104). cDNA was reverse transcribed with Superscript III (Invitrogen cat #18080), and RT-qPCR was performed. Relative gene expression was calculated by normalizing to Gapdh and are shown as 2−ΔCT (FIGS. 5C, 5D).

### Histology

Fractured tibiae were fixed in 4% paraformaldehyde (PFA), then decalcified in 19% ethylenediaminetetraacetic acid (EDTA) for 14 days. Mice were processed for paraffin histology through a graded ethanol series and cleared in xylene prior to embedding in paraffin tissue blocks. Serial sections were cut at 8-10 μm for histological analysis. Every 10th slide was stained with standard histological protocols for Hall and Brunt's Quadruple staining (HBQ) to visualize bone (red) and cartilage (blue) were used. Tissues from NGF-eGFP and Axin2-eGFP reporter strains were embedded in OCT and sectioned using a cryostat. Axin2-eGFP fluorescence was amplified by utilizing antibody conjugated to AlexaFluor488. X-Gal staining was performed as follows: samples were fixed in 4% PFA and after washing in PBS, samples were incubated in fresh X-Gal staining solution for 36 h at 32° C. After PBS washes, samples were post-fixed in 4% PFA at 4° C. for 16-24 h, decalcified, and embedded in OCT for cryosectioning and staining as previously described (Tomlinson, et al., 2017 PNAS USA 114:E3632-E3641).

### In Vitro Cartilage Explant Culture

Cartilage explants were isolated from the central portion of the day 7 fracture callus using a dissecting microscope to remove any adherent non-cartilaginous tissues. Explants were minced, pooled, then separated randomly into treatment groups. Explants were grown in vitro for one week in serum-free hypertrophic chondrogenic medium [high glucose DMEM, 1% penicillin-streptomycin, 1% ITS+Premix (BD Biosciences Cat #354352), 1 mM sodium pyruvate, 100 ng/ml ascorbate-2-phosphate and 10-7 M dexamethasone] to promote hypertrophic maturation (Bahney, et al., 2014, J Bone Miner Res 29:1269-1282; Hu, et al., 2017, Development 144:221-234). Hypertrophic cartilage explants were then stimulated with or without 200 ng/mL recombinant human β-NGF (Peprotech cat #450-01) for 24 h, collected in TRIzol, then mRNA was extracted using RNeasy Mini Kit following the manufacturer's instructions (Qiagen cat #74104).

### Gene Expression

Fracture calluses are dissected from the tibia and surrounding muscle. mRNA is then harvested using a standard TriZOL extraction technique. cDNA is reverse transcribed and qRT-PRC performed for the TrkA indicator (cFOS) (Sung, et al., 2018, J Neurosci 38:3394-3413) and standard chondrogenic (col2a1, col10a1, aggrecan) and osteogenic (col1a1, osteocalcin, osteopontin) markers using validated SyberGreen primers. Utilizing the mean and standard deviation from previously published qRT-PCR data set (Morioka, et al., 2019, Sci Reports 9:12199), a power analysis is conducted using GPower to determine that 3 mice/time are required to achieve significant power (power=0.8, a=0.05). 5 mice/group/time are included to account for potential additional variation associated with drug treatment. ANOVA and Tukey's HSD post-hoc testing are used to evaluate significance.

### RNA Sequencing and Analysis

After mRNA extraction from isolated fracture callus/hypertrophic cartilage, samples were then further purified by sodium acetate and isopropanol precipitation. 200 ng RNA input from each sample was used with Quantseq 3′ mRNA-seq Library Prep Kit FWD (Lexogen, SKU:015.24). Approximately 20 million single-end 50 bp reads were generated for each library on a HiSeq 4000. Reads were first trimmed for adapters with Cutadapt version 2.5 and then mapped to the mouse mm10 genome using STAR version 2.5.3a. Following alignment, reads were counted using featureCount version 1.6.4. Differential gene expression analysis was then performed using the DESeq2 package version 1.24 and R version 3.6.1. Significantly upregulated or downregulated genes (P<0.05, Benjamini-Hochberg corrected) upon treatment were entered into Enrichr (https://amp.pharm.mssm.edu/Enrichr/) for gene ontology classification. Differentially expressed genes and genes of interest were visualized using a combination of R, ggplot2 version 3.2.1, EnhancedVolcano, and Complexheatmap version 2.084.

### Immunohistochemistry (IHC)

β-NGF or control injections were administered once daily for 3 days beginning 7 days post-fracture into Axin2-eGFP mice. Fractured tibias were harvested 24 h after the final injection (10 days post-fracture), fixed in 4% paraformaldehyde (PFA) and decalcified in 19% ethylenediaminetetraacetic acid (EDTA) for 5 days. Samples were OCT-embedded then cryosections were made at a width of 8-10 μm. Cryosections were carefully rinsed in PBS and blocked with 5% bone serum albumin for an hour. Primary antibodies were applied to sections overnight. Species-specific secondary antibodies were detected using the VectaStain ABC Kit (Vector, PK-4000) and 3,3′-diaminobenzidine (DAB) colorimetric reaction was used to visualize CD31+ cells. Because of (d2)eGFP's rapid degradation, Axin2-eGFP fluorescence was stabilized by using species-specific Alexa-Fluor-488 conjugated secondary antibody (for example, host/target: rat anti-mouse CD31, goat anti-rat Ig (biotinylated), rabbit anti-GFP, goat anti-rabbit IgG).

### Histomorphometry

β-NGF and control injections were administered once daily for 3 days beginning 7 days post-fracture. Tibias were harvested 14 days post-fracture, fixed in 4% PFA and decalcified in 19% EDTA for 5 days. Mice were processed for paraffin histology, serial sections were cut at 8-10 μm for histomorphometric analysis using stereological principles. Quantification of callus composition (cartilage, bone, fibrous, marrow space) was determined using an Olympus CAST system (Center Valley, Pa.) and software by Visiopharm (Hørsholm, Denmark). For quantification of the tissues, 10 μm serial sections (three per slide) were taken through the entire leg. Tissue was stained with HBQ as described above, and the first section from every 10th slide analyzed such that sections were 300 μm apart. Volume of specific tissue types was determined in reference to the entire fracture callus by summing the individual compositions relative to the whole.

### Micro-Computed Tomography (μCT)

μCT analysis: fracture tibias were dissected free of attached muscle 14 days post-fracture, fixed in 4% PFA and stored in 70% ethanol. Fracture calluses were analyzed using the Scanco μCT50 scanner (Scanco Medical AG, Basserdorf, Switzerland) with 10 μm voxel size and X-ray energies of 55 kVp and 109 μA. A lower excluding threshold of 400 mg hydroxyapatite (HA)/mm³ was applied to segment total mineralized bone matrix from soft tissue in studies of control and β-NGF treated mice. Linear attenuation was calibrated using a Scanco hydroxyapatite phantom. The regions of interest (ROI) included the entire callus without existing cortical clearly distinguished by its anatomical location and much higher mineral density. μCT reconstruction and quantitative analyses were performed to obtain the following structural parameters: trabecular spacing (mm), trabecular number (#/mm), trabecular connective density as trabecular bifurcations (#/mm³), bone mineral density (mg HA/cm³), bone volume (as %), trabecular thickness (mm), and tissue mineral density (mg HA/cm³).

### Statistical Analysis

Individual dots on graphs represent biological replicates, error bars represent standard error of the mean (SEM). Measurements were taken from distinct samples. All in vivo data were analyzed using GraphPad Prism (version 8, GraphPad Software, San Diego, Calif.). Statistical tests used to compare between groups are specified in the corresponding figure legends, significant differences were defined at p<0.05.

### Results

### 2.1 NGF and TRKA are Expressed at the Chondro-Osseous Transition Zone During Endogenous Endochondral Fracture Repair

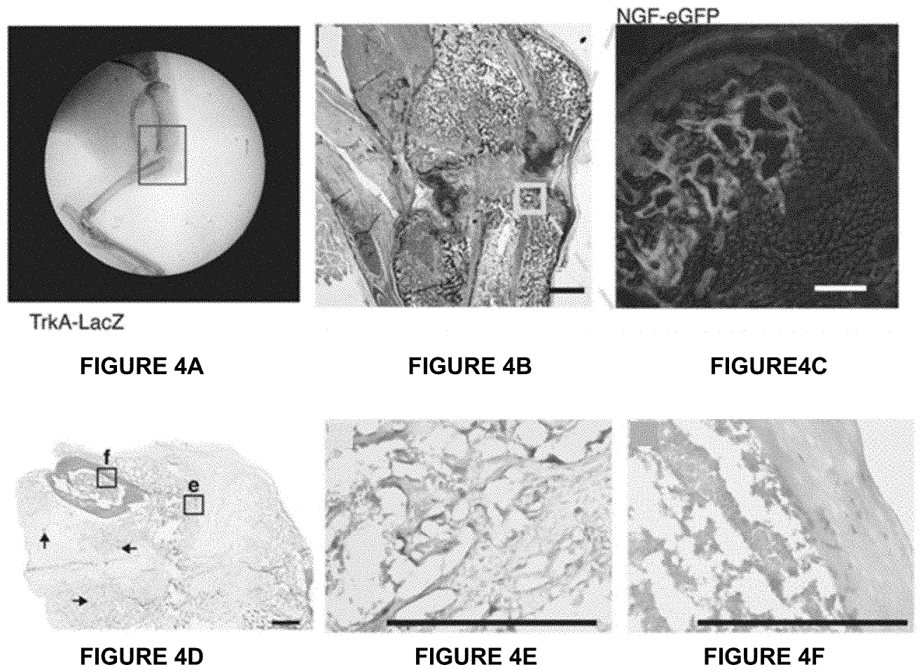

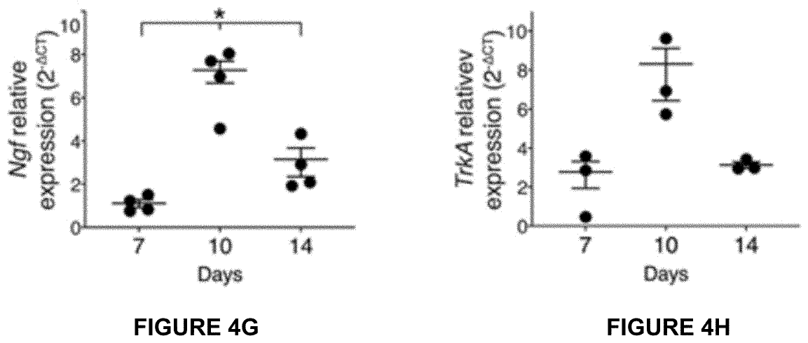

In order to determine the spatiotemporal parameters of endogenous NGF and TrkA expression during endochondral fracture repair in a murine model of long bone healing, closed fractures were created in the mid-shaft of the right tibia of adult male and female wild type mice (Jackson, 10-14 weeks old C57B16/J) using a well-established, three-point bending device to create closed, mid-shaft fractures in the right tibia of adult wild type mice (FIG. 4A). The custom-built apparatus was designed with a 2-cm blunt drop arm consisting of a 460 g weight, dropped a distance of ˜14 cm, to create the fracture without breaking the skin. These non-stabilized fractures generate a robust cartilage callus, as visualized by Hall and Brunt Quadruple (HBQ)-stained sections (cartilage=blue, bone=red) of tibiae harvested 14 days post-fracture, (FIG. 4B). By utilizing NGF-eGFP reporter mice, it was possible to visualize the expression domain of NGF within the chondro-osseous transition zone (TZ) of the fracture callus via fluorescence microscopy (FIG. 4C). TrkA expression appeared in fewer cells but could also be found within cells at this transition zone utilizing TrkA-LacZ reporter mice (FIGS. 4D-4F). After establishing the spatial expression patterns of NGF and TrkA at the TZ using histology, gene expression was used to define the temporal expression patterns of NGF and TrkA. Fracture calluses were isolated 7, 10, and 14 days following fracture, mRNA isolated using TRIzol, and RT-PCR used to quantify expression of NGF and TrkA. The data show similar temporal expression patterns of NGF and TrkA, with a peak 10 days post fracture (FIGS. 4G, 4H).

2.2 Endochondral Delivery of β-NGF is More Osteogenic than Early in Fracture Repair

The therapeutic efficacy of exogenous β-NGF in long bone fracture healing was tested. When developing novel therapies for fracture healing, the majority of drugs are given immediately after fracture by default. However, based on the endogenous spatiotemporal expression patterns of NGF-TrkA correlating with the conversion of cartilage to bone, it was investigated whether matching therapeutic delivery to the timing of this endogenous expression pattern would be more efficacious. Therefore, two different time points of β-NGF injections were tested: early, during the pro-inflammatory and intramembranous phase of repair (day 4-6, FIG. 5A), or later, during the endochondral phase of cartilage maturation (day 7-9, FIG. 5C). Local delivery was performed on isoflurane anesthetized animals by injecting 0.5 μg β-NGF, or basal media as a control. Early β-NGF injections resulted in significantly increased relative expression of collagen 1 (Col1) (FIG. 5B). However, there were significant decreases in osteogenic markers osteocalcin (Oc) and osteopontin (Op); and the pro-angiogenic vascular endothelial growth factor (Vegf) (FIG. 5B). Interestingly, later β-NGF injections robustly stimulated expression of osteogenic markers Oc and Op (FIG. 5D). Non-significant changes were observed in mRNA expression of Col1 (p=0.06) and Vegf (p=0.06) following β-NGF injections on the endochondral regimen (FIG. 5D).

2.3 β-NGF Stimulation of Fracture-Callus Derived Cartilage Explants Promotes Programs Associated with Endochondral Ossification

The molecular pathways activated by NGF in long bone fracture healing have not been studied. In order to understand the mechanism of action (MOA) for the painless NGF therapeutic, the TrkA receptor (TrkAfl/fl or p75^{NTR}fl/fl) is conditionally knocked out (KO) in either hypertrophic chondrocytes (Col10CreERT or Col2CreERT) or globally (R26CreERT2) after fracture to determine the extent to which this pathway is required for endochondral bone repair. Control mice or the KO mice are then treated with NGF^{R100W} to test whether chondrocytes mediate the trophic response to NGF^{R100W}. Using the combination of in vitro cell culture and RNAseq, which osteogenic pathways respond downstream to NGF-TrkA activation are investigated. The endogenous spatiotemporal expression patterns of NGF-TrkA in the TZ and enhanced osteogenic response of cartilage to β-NGF suggested that hypertrophic cartilage could be responsive to NGF.

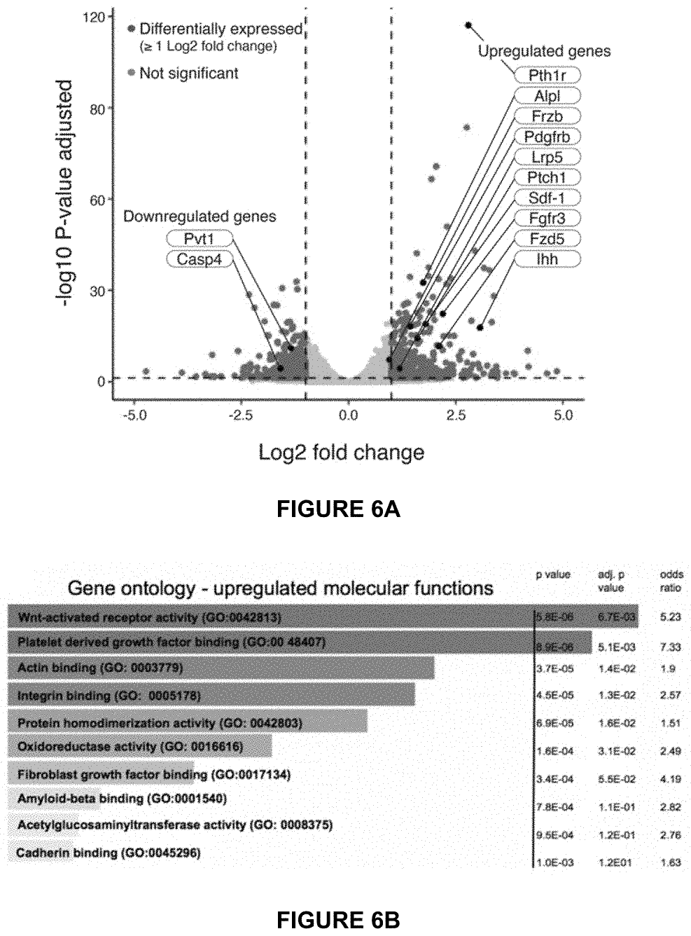

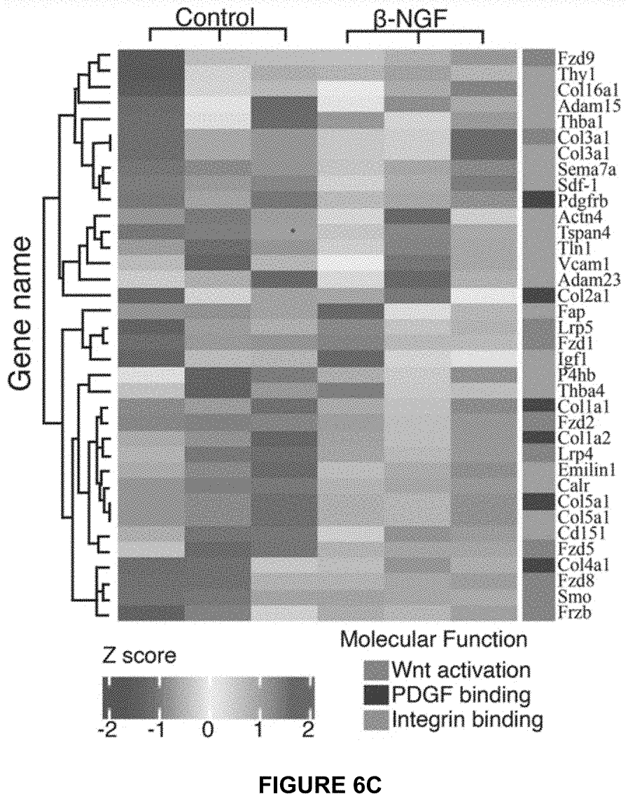

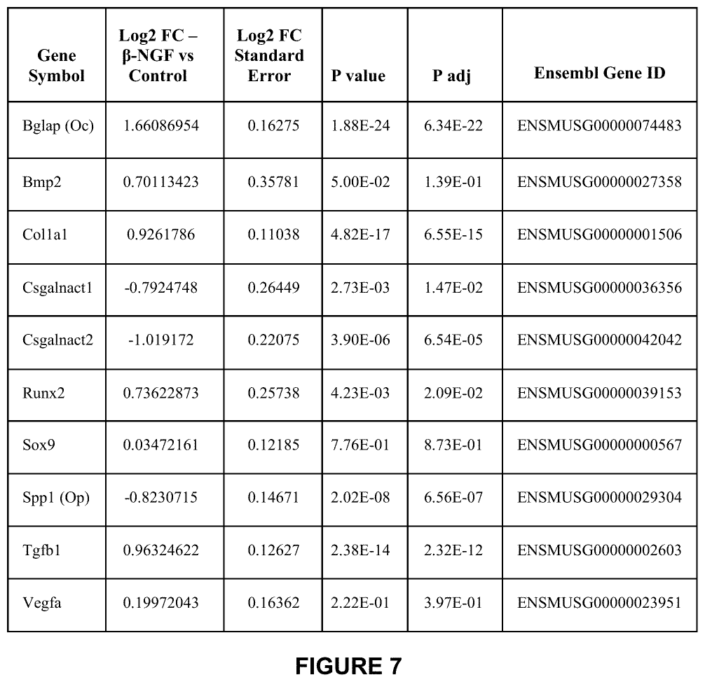

To test this, the cartilage was isolated from day 7 fracture calluses, as done previously (Bahney, et al., 2014, J Bone Miner Res 29:1269-1282; Hu, et al., 2017, Development 144:221-234). Explants were cultured to hypertrophy in vitro and treated with or without 0.5 μg/mL recombinant human β-NGF, the biologically active form of NGF40, for 24 h followed by RNA-sequencing (RNAseq). Similar to the in vivo study, the osteogenic marker Oc was significantly upregulated in the cartilage explant study (p=1.88E-24, FIG. 7). Additional analysis revealed a number of other significantly upregulated genes established to play a role in endochondral ossification, such as, Indian hedgehog (lhh), alkaline phosphatase (Alpl), parathyroid hormone 1 receptor (Pth1r), Wnt receptors (Lrp5, Frzd5) and angiogenic receptors (Pdgfrb) (FIG. 6A). Of the downregulated genes, plasmacytoma variant translocation 1 (Pvt1) and caspase 4 (Casp4) (FIG. 6A) are both known to modulate apoptosis.

Subsequent functional enrichment analysis using EnrichR showed multiple categories of molecular functions that were associated with endochondral ossification, fracture repair, and tissue remodeling. The three most significantly upregulated molecular function categories were: Wnt activation (p=0.0067), Platelet-derived growth factor (PDGF) binding (p=0.0051), and integrin binding (p=0.013) (FIG. 6B). With additional enrichment analysis, a heat cluster map of differentially expressed genes was created according to these molecular function categories (FIG. 6C).

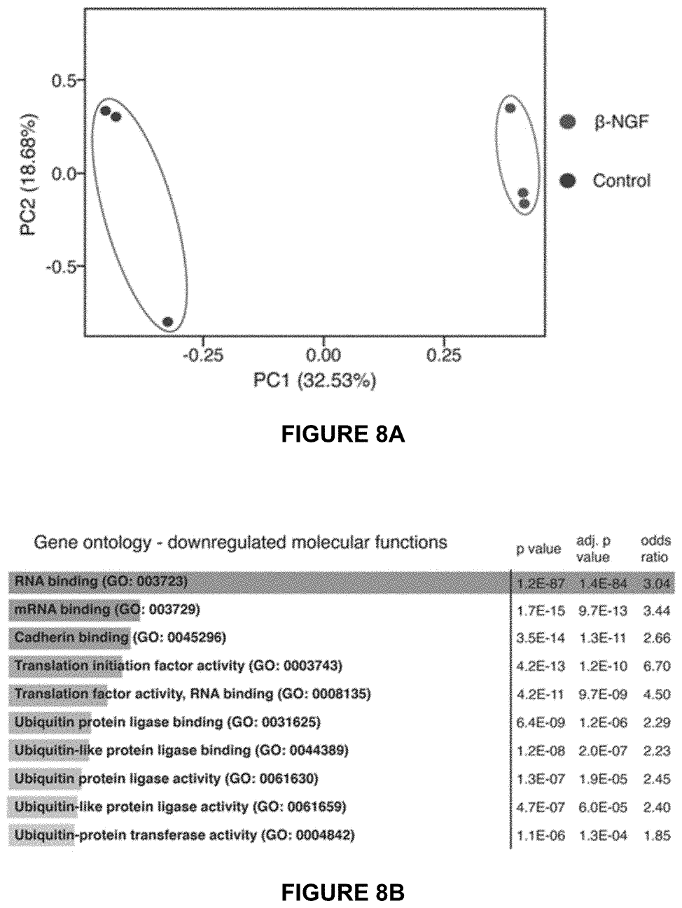

Data was generated for the enrichment analysis of β-NGF stimulated hypertrophic cartilage explants, including principal component analysis (PCA) for each biological replicate of β-NGF and non-stimulated controls (FIG. 8A) and gene ontology for downregulated molecular functions (FIG. 8B). Specifically, cartilaginous tissue was excised from tibia fracture 7 days post-fracture and cultured to hypertrophy for 7 days, then stimulated with or without recombinant human β-NGF. Samples were collected after 24 hours for RNAseq analysis (n=3). With additional enrichment analysis, a heat cluster map of differentially expressed genes was created according to these molecular function categories.

### 2.4 Evaluation of Wnt/β-Catenin Pathway Modulation Following NGF Delivery

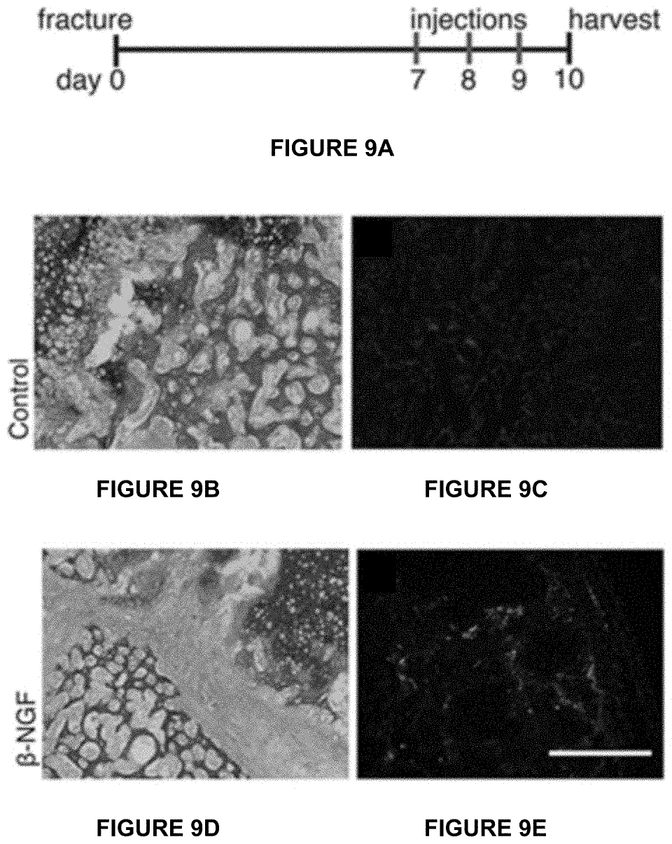

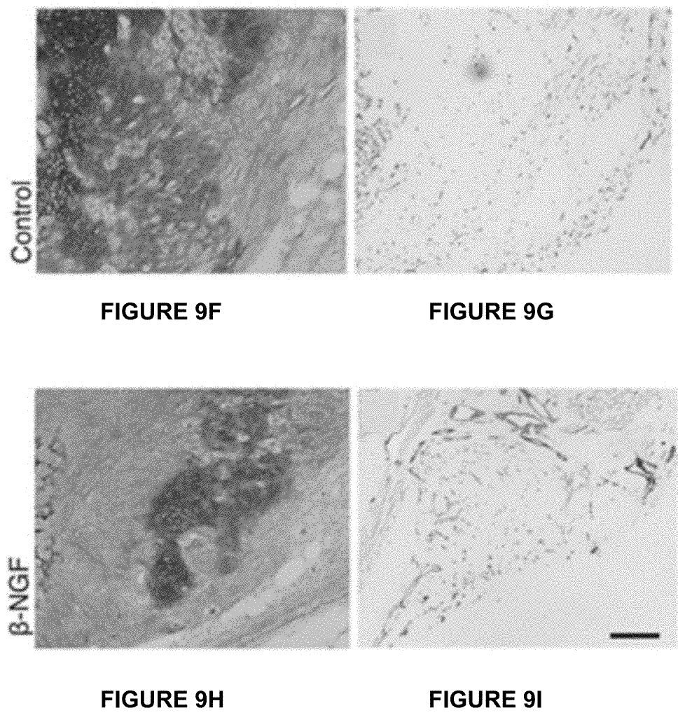

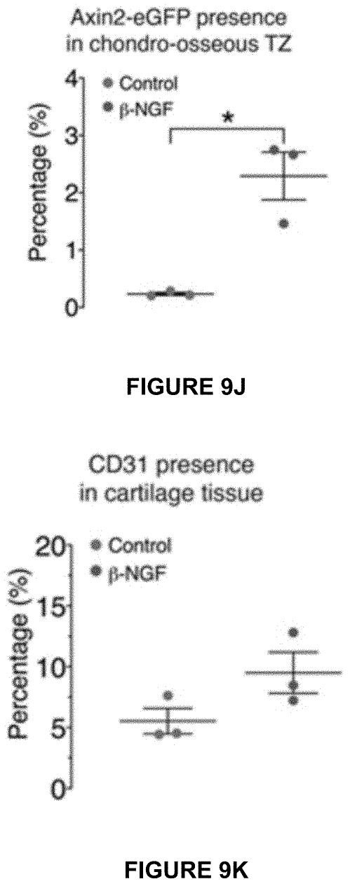

To confirm the RNAseq data suggesting that Wnt was the most significantly upregulated molecular function following β-NGF treatment of cartilage ex vivo (FIG. 6B, 6C)/to provide further evidence that NGF upregulates Wnt/β-cat signaling, a murine Axin2-eGFP reporter model was utilized to compare Wnt expression in vivo in mice treated with β-NGF to those without. Tibia fractures were made in the Axin2-eGFP mouse as described previously and β-NGF was injected locally into the fracture callus during the endochondral phase of repair, days 7-9 post-fracture (FIG. 9A). Visually, the control mice showed no major presence of Axin2-eGFP positive cells in the TZ (FIGS. 9B, 9C). However, there was an induction of Axin2-eGFP in cells at the TZ of β-NGF treated mice (FIGS. 9D, 9E). Quantification by Image-J confirmed the induction of Axin2-eGFP after β-NGF treatment compared with the lack of Axin2-eGFP presence in the control group (FIG. 9F). The association between NGF and Wnt/β-cat activation was significant in view of the finding that β-catenin expression in chondrocytes is critical to endochondral fracture repair (Wong, et al., 2020, bioRxiv 986141).

Given literature evidence that NGF signaling precedes and coordinates vascularization of bone tissue (Tomlinson, et al., 2016, Cell Rep 16:2723-2735), it was measured whether or not local β-NGF injections promoted the infiltration of endothelial cells into the cartilage callus. Angiogenesis was quantified using immunohistochemistry performed to the CD31 endothelial cell marker day 10 post-fracture in wild type mice that received the endochondral delivery of β-NGF. Vascular invasion to the cartilage callus was observed in both the controls (FIGS. 9G, 9H), with slightly more intense staining in the β-NGF group (FIGS. 91, 9J). Quantification by Image-J indicates only a nominal increase in CD31-positive cells in cartilage tissue of β-NGF treated mice (p=0.12) (FIG. 9K).

### 2.5 Local β-NGF Injections Accelerates Endochondral Bone Formation by 14 Days Post-Fracture

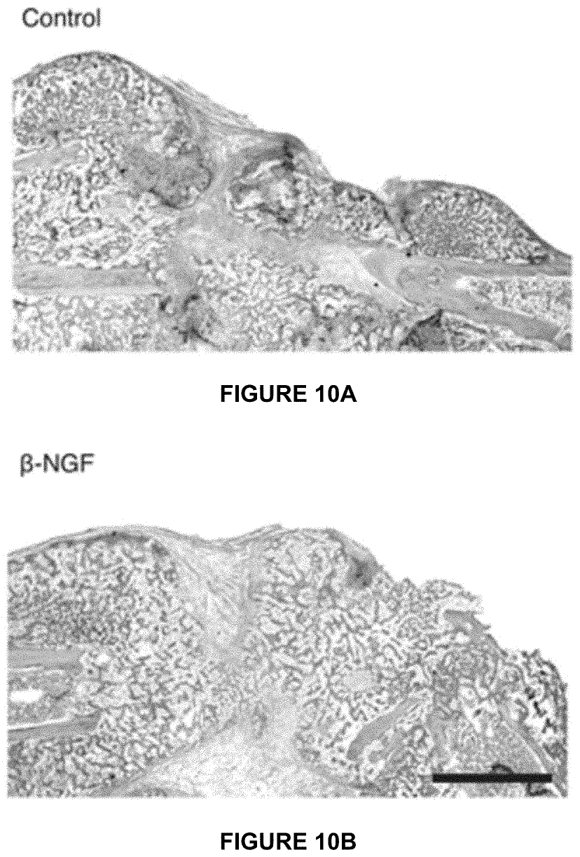

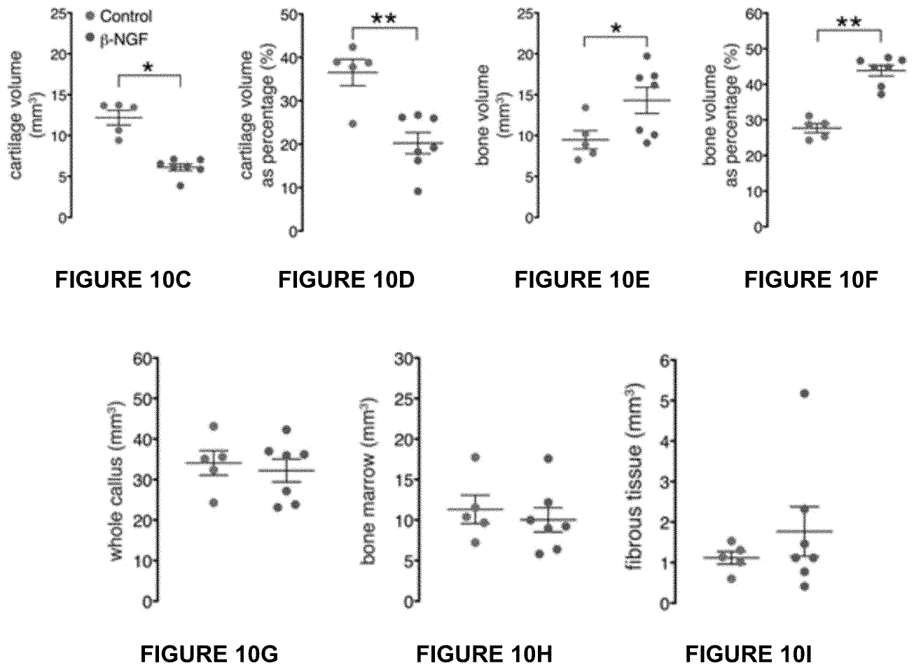

The functional outcomes of fracture healing with the endochondral delivery of therapeutic β-NGF were next tested using histomorphometric and quantitative μCT analysis on treated and control tibias 14 days post fracture. Histology clearly shows the increased formation of trabecular bone (red) and decreased cartilage (blue) in fractures receiving β-NGF relative to control (FIGS. 10A, 10B). Quantification of the cartilage tissue showed an almost 50% decrease in absolute volume (FIG. 100) and percent composition (cartilage volume/total volume) within the callus of β-NGF treated mice (FIG. 10D). Conversely, quantification of trabecular bone confirmed a similar increase in absolute bone volume (FIG. 10E) and composition (bone volume/total volume) of the callus after β-NGF treatment (FIG. 10F). There was no difference in volume of the callus as a whole between controls and β-NGF treated mice (FIG. 10G). There were also no differences in volume of bone marrow (p=0.59) (FIG. 10H) or fibrous tissue (p=0.40) between groups (FIG. 10I) suggesting that the conversion of cartilage to bone was accelerated in the experimental group.

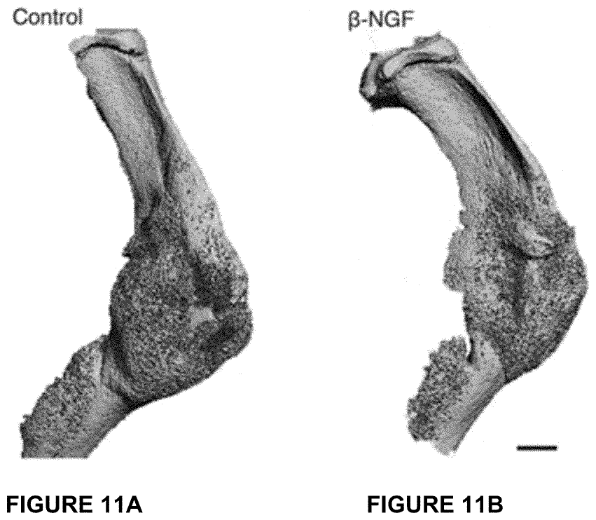

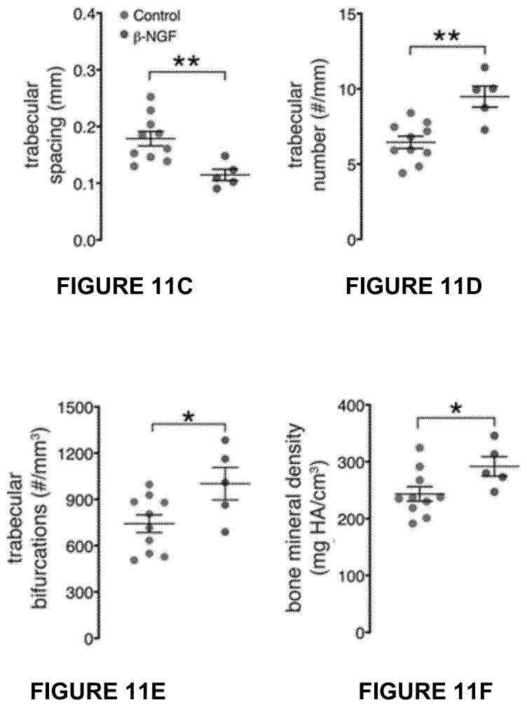

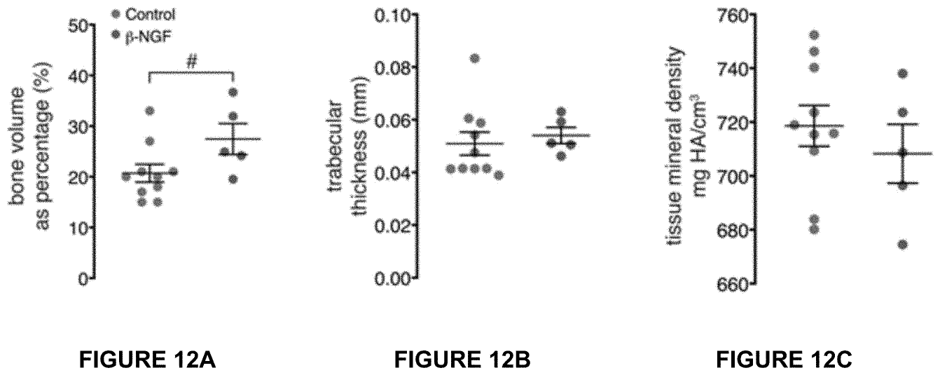

μCT analysis was performed in parallel to histomorphometry on control and β-NGF treated mice 14 days post fracture. Gross examination of μCT images provide no obvious differences between treatment groups (FIGS. 11A, 11B). However, quantitative assessment of structural indices showed a stark difference in bone architecture. β-NGF treated mice exhibited a 35% decrease in trabecular spacing compared to the controls (FIG. 11C), with trabecular number (FIG. 11D) and trabecular connective density showing dramatic increases of over 40% (FIG. 11E). Bone mineral density measurements also significantly increased, 20%, in the fracture callus of β-NGF treated mice (FIG. 11F). Further results for the μCT analysis of trabecular bone within fracture callus are shown in FIGS. 12A-12C. Local injections of media (control) or 0.5 μg β-NGF were administered once daily at 7, 8, and 9 days post-fracture (p.f.), tibias were then harvested 14 days p.f. for μCT analysis. Taken together, μCT data depict highly connected and structurally superior bone architecture in β-NGF treated mice indicative of a later stage of endochondral repair.

Thus, β-NGF was most efficacious in promoting long bone fracture healing when the drug was administered during the cartilaginous phase of repair, days 7 to 9 post fracture, reflecting the upregulation in endogenous Ngf and TrkA gene expression observed. Histological data utilizing NGF-eGFP and TrkA-LacZ reporter mice provide the first genetic labeling of the expression pattern within the callus of tibia fractures. NGF and TrkA were localized predominantly at the chondro-osseous transition zone, where cartilage undergoes hypertrophy and transforms to bone adjacent to the invading vasculature.

The genetic models of endogenous NGF and TrkA localization support observations of peak expression of neurotrophins and their receptors during the hypertrophic cartilage phase of repair (Asaumi, et al., 2000, Bone 26:625-633; Sun, et al., 2020, Bone 131:115109; Grills and Schuijers, 1998, Acta Orthop Scan 69:415-419). While the mRNA expression data show a peak at day 10, possible delays in mRNA-to-protein synthesis were considered when harvesting samples at day 14 for histological analysis. Histological visualization of NGF and TrkA expression at this timepoint demonstrate a broad and robust presence in the chondro-osseus transition zone of tibial fracture calluses.

The osteogenic transformation of chondrocytes into osteoblasts during bone development and fracture repair is associated with the upregulation of traditional programs regulating osteogenesis and mineralization. Through gene ontology, molecular functions associated with Wnt activation were identified as those most significantly upregulated following β-NGF treatment. The instant example additionally constitutes the first study in which the relationship between NGF signaling and Wnt activation has been noted in cartilage after in vitro stimulation with β-NGF. Wnt activation was also confirmed in vivo by histological analysis using Axin2-eGFP mice following local β-NGF injections. Thus, β-NGF treatment likely stimulates Wnt-mediated cartilage to bone conversion during endochondral fracture repair.

It was additionally shown in the instant example that timing of injections is important in dictating the best therapeutic window of β-NGF. A stronger osteogenic effect of β-NGF was found when delivered during the endochondral phase of repair, as opposed to early, during the pro-inflammatory response and intramembranous healing. With endochondral delivery β-NGF, histomorphometric analyses of callus tissue resulted in a reduction in cartilage and increase in bone tissue compared to control. Furthermore, no change in the total volume of the fracture callus was noted, supporting the finding that β-NGF accelerates cartilage to bone conversion. In addition to histomorphometry, μCT data further illustrated the high connectivity and high mineral density of the newly formed trabeculated bone.

Indeed, gene expression analysis, histomorphometry, and μCT data collectively demonstrated that β-NGF treatment during the endochondral phase of fracture repair stimulates osteogenesis to produce more bone tissue, and that the newly formed bone is more connected and of higher architectural quality. The limitation on NGF's clinical translation lies in its hyperalgesic effects.

### Example 3. Therapeutic Delivery of Nerve Growth Factor Accelerates Cartilage to Bone Conversion During Fracture Healing

Bone fractures heal primarily through the process of endochondral ossification. Endochondral ossification, or indirect bone formation, occurs when cartilage forms between bone gaps and is later replaced by bone. The conversion of cartilage to bone in the fracture callus occurs adjacent to the invading neurovascular bundle. While angiogenesis and associated factors have been heavily studied during endochondral ossification, there is limited work exploring a role for neuronal signaling in this process. Because vascularization and neuralization of the hypertrophic chondrocyte zone are both critical for proper post-natal bone development, it was investigated whether an induction of nerve growth factor (NGF) expression and its receptor TrkA occurs in the callus during fracture healing, and whether local administration of NGF would enhance fracture repair by promoting cartilage-to-bone-transformation in fractured tibias of mice.

### Methods

NGF expression during fracture healing: closed and unstabilized fractures were made at the mid-shaft of the right tibia using a three-point fracture device on 10-14 week old male C57Bl6/J wildtype mice. Calluses were harvested on days 7, 10, and 14 post-fracture to measure neurotrophic gene expression. Samples were collected in Trizol to isolate mRNA for RT-qPCR (N=4).

NGF effects on gene expression: NGF (0.5 ug in 20 uL DMEM) injections into fracture calluses began either 3 or 7 days post-fracture for 3 consecutive days. Controls were injected with only DMEM. Fracture calluses were then harvested 24 hours following the final NGF injection and collected in Trizol to then isolate mRNA for RT-qPCR analysis (N=3) of osteogenic and chondrogenic markers. Significant differences in mRNA expression were determined using an ANOVA followed by post hoc Tukey-Kramer HSD (a=0.05).

NGF effects on fracture callus tissue composition: NGF and control injections were done on days 7-9 post-fracture, and then tibias were harvested 14 days post-fracture. Fractured tibiae were fixed in 4% paraformaldehyde for 24 h, then decalcified in 19% EDTA for 14 days at 4° C. then processed for paraffin embedding and stereological analysis (Hu, et al., 2017, Dev 144:221-234). Serial sections were cut at 10 μm, and Hall and Brunt Quadruple stain (HBQ) protocol was used to visualize bone (red) and cartilage (blue). Quantification of callus composition was determined using an Olympus CAST system and software by Visiopharm (N=8). Significance was determined using Wilcoxon/Kruskal-Wallis test (a=0.05).

### Results

An induction of angiogenic and neurotrophic gene (TrkA, VEGF, NGF) expression during fracture healing was observed, with a peak in expression on day 10 post-fracture (data not shown). After NGF injections during either the intramembranous phase of fracture healing (day 3-5), or during the endochondral phase of fracture healing (days 7-9), quantification of osteogenic gene expression 24 h following the last injection shows strong activation of col I, osteocalcin (oc), and osteopontin (op) only with injections during cartilaginous phase of repair relative to the DMEM-treated controls (data not shown). When injected during the intramembranous phase of healing, only col I expression differed in the callus of NGF-treated mice compared to control, but when injected during the endochondral phase, col 2, col I, oc, and op expression differed in the callus of NGF-treated mice compared to control. When NGF is delivered at this later time-point, histology shows more newly formed trabecular bone, and less cartilage 14 days post-fracture. Stereological quantification revealed no difference in callus size between groups, however, bone volume was significantly higher in NGF treated mice translating to an increased percentage of bone in the fracture callus (data not shown). Conversely, cartilage volume and composition were significantly lower in NGF-treated mice (data not shown).

Thus, NGF and TrkA expression was induced in tibiae during days 7-14 post-fracture. Additionally, VEGF expression also peaked at day 10 post-fracture in parallel with NGF/TrkA signaling. RT-qPCR data of NGF-treated fractures showed a more robust promotion of osteogenic markers in the cohort treated during the cartilaginous phase of endochondral repair (days 7-9 post-fracture). This resulted in increased amount of bone and decreased in cartilage 14 days post-fracture, compared to the control group, indicating that NGF delivery accelerated the conversion of cartilage to bone. This local NGF administration appears to activate osteogenesis in the cartilage callus.

### Example 4. Localized Delivery of β-NGF Via Injectable Microrods Accelerates Endochondral Fracture Repair

### PEGDMA Microrod Fabrication

In order to determine whether engineered PEGDMA microrods accelerate endochondral repair by generating sustained release of NGF, microrods were fabricated as previously described (Ayala, et al., 2010, Tissue Engineering Part A 16:2519-2527). Briefly, poly(ethylene glycol) dimethacrylate (PEGDMA) molecular weight 750, photoinitiator 2,2-dimethoxy-2-phenylacetophenone (DMPA) were dissolved in 1-vinyl-n-pyrrolidone (NVP) to a concentration of 100 mg/mL in phosphate-buffered saline (PBS). This solution was sonicated at room temperature for 15 minutes to homogenize. 25, 75, and 90% PEGDMA microrods were created by varying the % v/v PEGDMA to PBS. Photolithography was used to create microrods designed to have the dimensions 100×15×15 μm, micro-fabricated on 3-inch silicon wafers using previously established methods (Yang, et al., 2014, PNAS USA 1302703111). Briefly, wafers were cleaned in piranha solution (3:1 H2SO4:H2O2) for 20 mins and rinsed 3 times with DI-H2O. Wafers were then rinsed with acetone, methanol, and isopropanol; then baked for 2 min at 200° C. The PEGDMA precursor solution was deposited onto each wafer wherein the wafers had a 15 μm-deep bevel prefabricated with SU-8 2015. The wafer was exposed using a Karl Suss MJB3 mask aligner to a 405 nm light source through a microrod patterned photomask at 9 mW/cm². Microrods on the wafer were rinsed and removed with 70% ethanol while gently scraping with a cell scraper. The collected microrods were centrifuged and rinsed in 70% ethanol three times before being resuspended in sterile deionized water (diH2O) with 10% sucrose and 0.05% tween-20 to prevent aggregation. Aliquots of ˜100,000 PEGDMA microrods were then lyophilized, sealed, and stored at 4° C. until further use. A subset of PEGDMA microrods was resuspended in PBS and micrographed under bright field (BF). Another subset was stained with the low molecular weight dye 4′,6-diamidino-2-phenylindole (DAPI) 1 μg/mL in PBS for 5 mins, washed with PBS gently three times, then immediately imaged using a Nikon Ti microscope.

### Protein Loading of PEGDMA Microrods

Bovine α-Chymotrypsinogen A (Sigma) was used as a proxy for comparing loading efficiencies between 25, 75, and 90% PEGDMA microrods, as its molecular weight (25.7 kDa) is similar to that of β-nerve growth factor (β-NGF, 27 kDa). Lyophilized PEGDMA microrods aliquots (˜100,000 microrods/aliquot) were resuspended in 20 μL of 1 mg/mL α-Chymotrypsinogen A in diH₂O. After resuspension, the microrods were allowed to passively adsorb protein for 30 hours in 4° C. After loading, microrods were resuspended in diH2O, gently spun down to pellet in tube, and the supernatants were used to perform a micro bicinchoninic acid (μBCA) protein assay to quantify the amount of protein left in solution. To determine loading efficiency, the following equation was used: Loading efficiency %=((Xᵢ−Xₜ)/Xᵢ)*100; where Xₜ is amount of protein in the supernatant after 30 hours and Xᵢ is the quantity of drug added initially during preparation. Loading of PEGDMA microrods with β-NGF and calculation of loading efficiency was done similarly in subsequent assays.

NGF was loaded into the microrods through absorption by placing them at a concentration of 1×10⁶/mL in a 1 mg/mL NGF-PBS solution for 24 hours in 4° C. NGF-loaded microrods were then collected by centrifugation at 4° C., and absorption efficiency was calculated through [NGF] in the supernatant. To calculate release kinetic, microrods were then re-suspended (1×10⁶ microrods/mL) in 100 μL of PBS aliquots, agitated gently at 37° C., with supernatant collected at days 1, 3, 5, 7, and 14, and protein content was measured with the micro-BCA protein assay. A release rate of 0.50 μg/day for 7 days was targeted.

### Erythroblast (TF-1) Cell Proliferation

TF-1 cell proliferation assay was modified from the established method (Ma and Zou, 2013, J Applied Virol 2(2):32). Briefly, TF-1 cells (ATCC) were cultured for 7 days in RPMI 1640 Medium modified with 2 mM L-glutamine, 10 mM HEPES, 1 mM sodium pyruvate, 4500 mg/L glucose, and 1500 mg/L sodium bicarbonate (ATCC 30-2001) supplemented with 2 ng/ml recombinant human Granulocyte-Macrophage Colony-Stimulating Factor (Sigma-Aldrich) and 10% fetal bovine serum. Following 7 days of cell growth, confluent TF-1 cells were collected by centrifugation and resuspended into 24-well microplates with 30,000 cells per well containing 600 μl of serum-free medium. Cells were cultured in serum-free medium for 24 hours to synchronize the cells prior to adding treatment groups. After 24 hours, high pore density (0.4 micron) transwell inserts containing either 100 μl of serum-free medium, 16,000 empty microrods, 2000 ng of soluble β-NGF, or 16,000 microrods containing 18 ng of β-NGF were inserted into each well and cultured in these conditions for an additional 96 hours. A 24-well plate containing 30,000 cells per well was removed and analyzed (see below) after the 24-hour serum-free medium incubation as a Day 0 control. After 96 hours, the transwell inserts were removed and 300 μl from each well was aspirated. The 300 μl collected was aliquoted into an individual well in a 96-well microplate containing 100 μl each (3 wells total for a single well in the 24 well plate) and subjected to a CyQuant© Direct Proliferation Assay Kit (Thermo Fisher) per the manufacturers protocol. Data is represented as a fold change relative to the cell number at Day 0.

Confirmation of Bioactivity of NGF Eluted from Microrods

In order to assess the bioactivity of NGF after loading and release from PEGDMA microrods, a previously established TF1 proliferation assay was used (Malerba, et al., 2015, PLoS ONE 10:e0136425). TF1 cells (that express TrkA) were cultured for 1 week in DMEM with 10% fetal bovine serum (FBS) with 2 ng/mL rhGM-CSF. After culture, cells were plated on 96-well microplate at a concentration of 300,000 cells/mL (15,000 cells per well in 50 μl). 60 min after plating, cells were exposed to NGF-microrods supernatants in DMEM 10% FBS. After 40 hours of incubation, 60 μl of the reagent CellTiter 96 Aqueous 1 Step Solution Reagent (Promega Corporation, Madison, USA) were pipetted into each well. Following an additional hour of incubation, absorbance at 490 nm were recorded using an Elisa plate reader (Wallac Victor V21420 spectrophotometer). NGF released from PEGDMA microrods increased proliferation of TF1 cells (data not shown).

β-NGF Elution from PEGDMA Microrods

Lyophilized 90% PEGDMA microrods were loaded by resuspending ˜100,000 (100 k) microrod aliquots in 20 μL of 1 mg/mL recombinant human β-NGF (Peprotech). After loading, microrods were gently rinsed thrice with PBS. PEGDMA microrods were then further divided, samples consisted of 16 k microrods/microtube suspended in 250 μL of PBS (pH 7.4). Samples placed onto an orbital shaker (100 RPM) within an incubator (37° C.). PEGDMA microrods were spun down gently and the supernatants were collected and replenished at 6, 24, 48, 72, 96, 120, 144, and 168 hours. Collected supernatants were immediately flash frozen and stored at −80° C. until further use. ELISAs for human β-NGF (RayBiotech) were performed per the manufacturer's instructions and daily release amounts were calculated by an established standard curve.

### Determination of Therapeutic Efficacy In Vivo of NGFR100W-Eluting PEGDMA Microrods

Tibia fractures and fracture callus composition: for preclinical validation of this system, the murine tibial fracture model that promotes robust endochondral ossification is utilized (Hu, et al., 2017, Development 144:221-234; Le, et al., 2001, J orthopaed res 19:78-84; Lu, et al., 2006, The Iowa orthopaedic journal 26:17-26); closed fractures are made at the midshaft of the right tibia using a three-point fracture device on 10-14 week old C57Bl6/J wild-type mice. Four treatment groups are (1) DMEM, (2) NGF^{R100W} in DMEM, (3) microrods in DMEM, and (4) NGF^{R100W}-loaded microrods in DMEM. Since preliminary data showed that NGF administration was most effective starting 7 d post-fracture, during the cartilaginous phase of repair, treatments are injected percutaneously into the fracture callus once, 7 d post-fracture. Tissues are harvested at days 14, 21, and 28 after fracture for functional assessment (n=10/time/group, n=120 total). Fracture callus composition is quantified by stereology. Briefly, the callus is fixed in 4% paraformaldehyde, decalcified in 19% EDTA for two weeks, and processed for paraffin embedding. Serial sections (10 μm) are mounted on slides and stained with Hall Brunt Quadruple (HBQ). Tissue is quantified using the automated Olympus Cast system and Visopharm software.

### Murine Tibial Fracture Model

Studies were conducted on the C57BL6/J wild type strain obtained from Jackson Labs (Stock #000664). Briefly, adult (10-16 weeks) male mice were anesthetized via inhalant isoflurane, and closed non-stable fractures were made mid-diaphysis of the tibia via three-point bending fracture device (Bahney, et al., 2014, J Bone Mineral Res doi:10.1002/jbmr.2148). Fractures were not stabilized, as this method promotes robust endochondral repair. After fractures are created, animals were provided with post-operative analgesics (buprenorphine sustained release). Animals were socially housed and allowed to ambulate freely.

### Local Injections

Percutaneous injections into tibial fracture calluses of mice were administered 7 days post-fracture. A precise microliter Hamilton© syringe was utilized for all injections wherein experimental agents were injected in 20 μL of PBS. Experimental groups are as follows: Controls injected with sterile PBS, β-NGF group injected with 500 ng of β-NGF in PBS, non-loaded microrods group injected with 16,000 PEGDMA microrods in PBS, and β-NGF microrods group injected with 16,000 PEGDMA microrods loaded with 18 ng of β-NGF.

### Biomechanical Testing and μCT Analysis

To obtain comprehensive analysis of fracture healing outcomes, based on stereology outcomes, the time point with greatest difference in callus composition is selected to complete a pilot study including biomechanical strength testing and μCT analysis (presumably 14 or 21 d post fracture, n=10/group, n=40 total). For these samples, tibias are harvested and transferred to 70% ethanol for μCT analysis on the Scanco μCT50 scanner in the UCSF Skeletal Biology Core (“SBB”) to determine bone mineral density (BMD) and bone volume (BV). Subsequently samples are transferred to the CCMBM Skeletal Biology Biomechanics Core for 3-point bending (Bose Corp., Eden Prairie, Minn., USA).

### Micro-Computed Tomography (μCT)

μCT analysis was performed as previously described (Cheng, et al., 2020, J Bone Mineral Res doi:10.1002/jbmr.3864; Hu, et al., 2017, Dev (Cambridge) 144(2):221-234). Fracture tibias were dissected free of attached muscle 14 days post-fracture, fixed in 4% PFA and stored in 70% ethanol. Fracture calluses were analyzed in the UCSF Core Center for Musculoskeletal Biology (CCM BM, NIH P30 funded core) using the Scanco μCT50 scanner (Scanco Medical AG, Basserdorf, Switzerland) with 10 μm voxel size and X-ray energies of 55 kVp and 109 μA. A lower excluding threshold of 400 mg hydroxyapatite (HA)/mm³ was applied to segment total mineralized bone matrix from soft tissue in studies of control and treated mice. Linear attenuation was calibrated using a Scanco hydroxyapatite phantom. The regions of interest (ROI) included the entire callus without existing cortical clearly distinguished by its anatomical location and much higher mineral density. μCT reconstruction and quantitative analyses were performed to obtain the following structural parameters: volume fraction (bone volume/total volume as %), trabecular connective density as trabecular bifurcations (#/mm³), and bone mineral density (mg HA/cm³).

### Localization Histology

Tibias were harvested 12,14, and 21 days post-fracture (5, 7, or 14 days post injection of microrods) to observe microrod localization. At time of collection, tibias were fixed in 4% PFA and decalcified in 19% EDTA for 14 days at 4° C. with rocking and solution changes every other day. Tibia were processed for paraffin embedding, serial sections were cut at 10 μm (3 sections per slide), and Hall Brundt's Quadruple (HBQ) staining protocol was done to visualize the bone (red) and cartilage (blue) as previously described to localize PEGDMA microrods (Rivera, et al., 2020, Sci Reports 10:22241; Hu, et al., 2017, Dev (Cambridge) 144(2):221-234). The sections were mounted on slides with Permount™ mounting medium and brightfield images were captured on a Leica DMRB microscope.

### Histomorphometry

Fracture callus composition was determined using quantitative histomorphometry of tibia harvested 14 days post-fracture. Standard principles of histomorphometric analysis were utilized to quantify the bone and cartilage fraction in the fracture callus using the first section from every 10th slide analyzed, such that sections were 300 μm apart. Images were captured using a Nikon Eclipse Ni-U microscope with Nikon NIS Basic Research Elements Software version 4.30. Quantification of callus composition (cartilage, bone, fibrous tissue, background) was determined using the Trainable Weka Segmentation add-on in Fiji ImageJ (version 1.51.23; NIH, Maryland, USA)50. Volume of specific tissue types was determined in reference to the entire fracture callus by summing the individual compositions relative to the whole.

### Statistical Analysis

Individual dots on graphs represent biological replicates, error bars represent standard error of the mean (SEM). Measurements were taken from distinct samples. Data were analyzed using GraphPad Prism (version 8, GraphPad Software, San Diego, Calif.). ANOVA was used to determine statistical differences between multiple groups followed by Tukey's HSD post-hoc comparison testing. Significant differences were defined at p<0.05.

### Results

### 4.1 Injectable PEGDMA Microrod Fabrication Via Photolithography

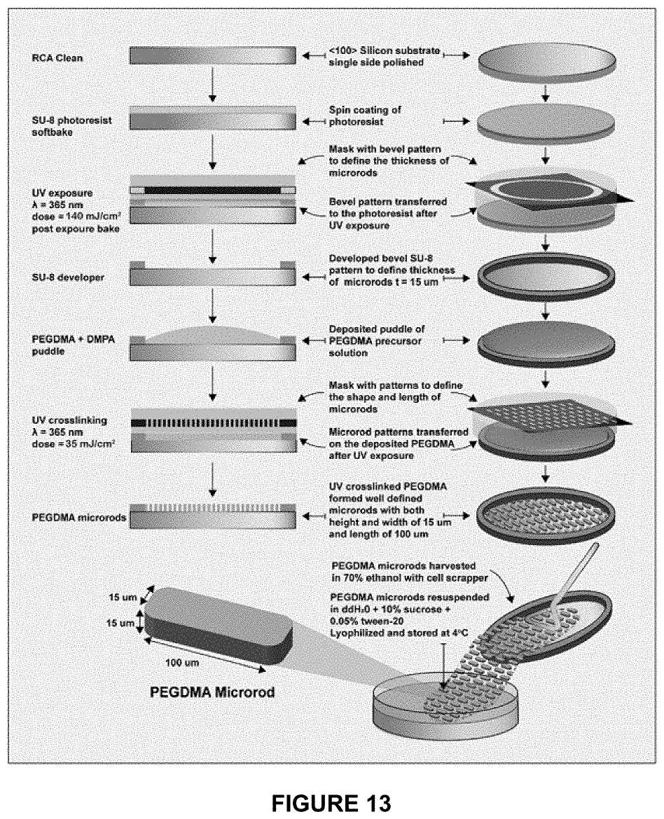

As mentioned earlier, PEGDMA microrods were fabricated through a process of photolithography (FIG. 13). The exact dimensions of the PEGDMA microrods can be carefully controlled by the photomask (length and width). The microrod height is determined by distance between the silicon wafer and the photomask which is manually controlled by use of the mask aligner. PEGDMA microrods are then cross-linked with UV irradiation, detached with a cell scraper from the silicon wafer and collected. Each individual PEGDMA microrod had the following dimensions: H=15 μm, W=15 μm, and L=100 μm. The 3D structure of the microrods is formed through free radical chain photopolymerization of the methacrylate groups at each end of each 750 MW PEG monomer unit. This system provides a high-throughput method to produce highly uniform PEGDMA microrods.

### 4.2 PEGDMA Microrod Macromer Concentration Changes Protein-Loading Efficiency

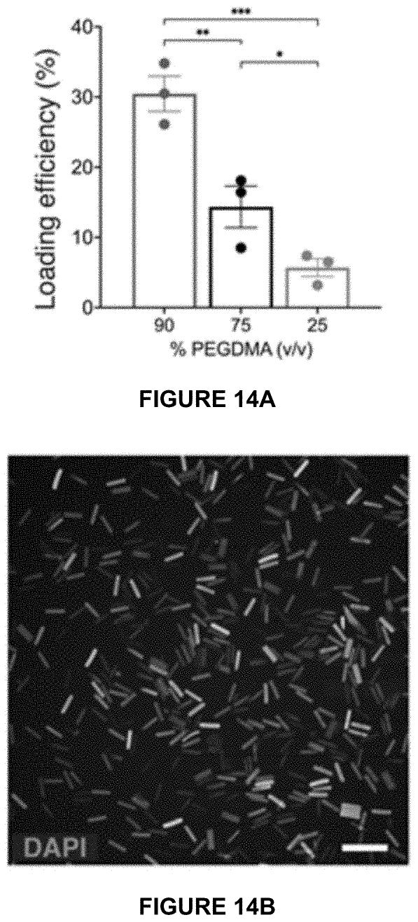

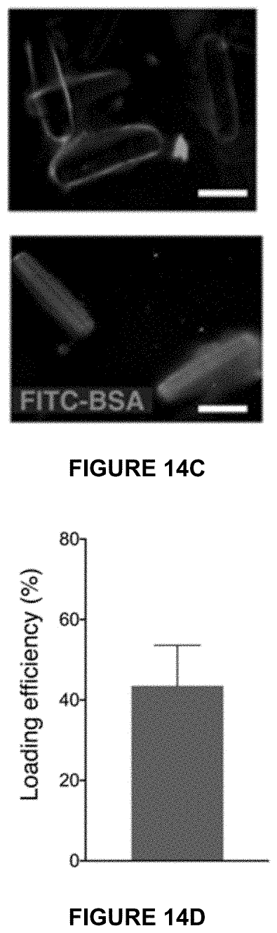

The first goal was to tune the PEGDMA microrod polymer network density to maximize protein loading efficiency. Prior to photolithography, the PEGDMA macromer concentration was adjusted to contain low (25%), medium (75%), or high (90%) volume (v/v) amounts (FIG. 14A). After fabrication, the PEGDMA microrods were lyophilized (freeze dried) to remove any residual liquid remaining that could alter protein loading and then loaded with α-Chymotrypsinogen A as a proxy protein, as its molecular weight is similar to that of β-NGF (26 kDa). The low and medium macromer volume PEGDMA microrods had modest loading efficiencies of less than 5% and 15%, respectively. The high macromer volume PEGDMA microrods exhibited a significantly larger loading efficiency, over 30%, when compared to the other PEGDMA concentrations. Since the 90% PEGDMA (v/v) microrods had the best loading efficiency, this formulation was chosen for all the subsequent experiments. β-NGF loading efficiency was then confirmed by loading high macromer PEGDMA microrods with β-NGF which resulted in over 40% loading efficiency (FIG. 14D). DAPI, a commonly used nuclear counter stain, can be easily adsorbed into the PEGDMA microrods and visualized with fluorescent microscopy (FIG. 14B). The DAPI-stained microrods are uniform in size and do not exhibit any aggregation, indicating good dispersity in solutions to allow for more optimal protein loading. Given that protein loading is driven by physisorption, the absorption of proteins onto the microrods and the rate at which protein elution occurs after loading were qualitatively examined. Using FITC-BSA as a model protein, the superficial layer of the PEGDMA microrods are coated with the fluorescently-tagged protein with no diffusion at time 0 (FIG. 14C, Top). After 60 minutes of incubation, diffusion is drastically increased and FITC-BSA can be observed eluting in the surrounding space of the PEGDMA microrods (FIG. 14C, Bottom).

4.3 Bioactivity Retention and Sustained Release of β-NGF from PEGMDA Microrods

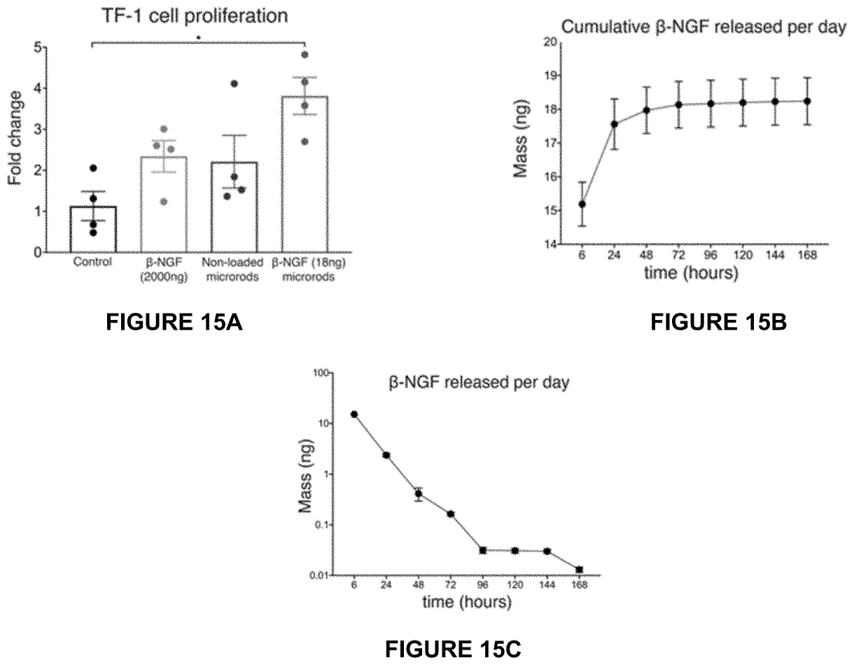

Whether β-NGF retained bioactivity when released from the PEGDMA microrods was tested next. To do this, the erythroleukemia Trk-A expressing cell line, TF-1, was utilized in the presence of culture media (control), 2000 ng of soluble β-NGF, non-loaded microrods, or 16,000 PEGDMA microrods loaded with 18 ng of β-NGF (FIG. 15A). Sustained release from β-NGF microrods was hypothesized to increase proliferation of TF-1 cells relative to the other treatment groups following 4 days of culture. Soluble NGF-treated cells exhibited a 2-fold increase in proliferation relative to the control. The non-loaded microrods also exhibited a similar increase in proliferation as soluble NGF-treated cells. However, the hypothesis was supported by the statistically significant 4-fold increase in proliferation by the β-NGF microrods. The increase in proliferation may be attributed to the sustained release of β-NGF observed over a 168-hour (7 day) period (FIGS. 15B, 15C). The β-NGF microrods exhibited an initial burst release of β-NGF within the first 24-48 hours, followed by sustained release over the next 120 hours (days 2-7), as measured by ELISAs (FIG. 15B). The total daily amount of eluted β-NGF decreased with time indicating concentration dependent (first order) release kinetics from the PEGDMA microrods. Nonetheless, elution of β-NGF was detected and quantified over a 7-day period (168 hours) (FIG. 15C).

### 4.4 β-NGF Loaded PEGDMA Microrods Promote Endochondral Bone Formation

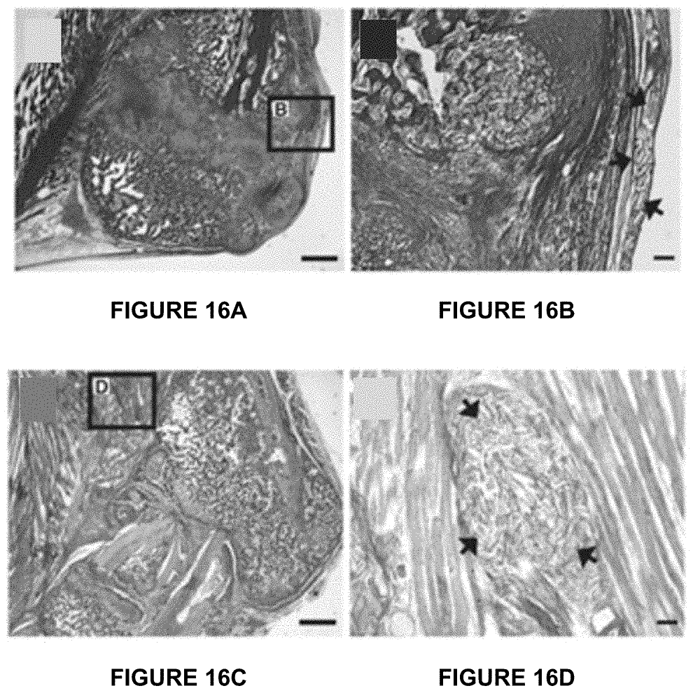

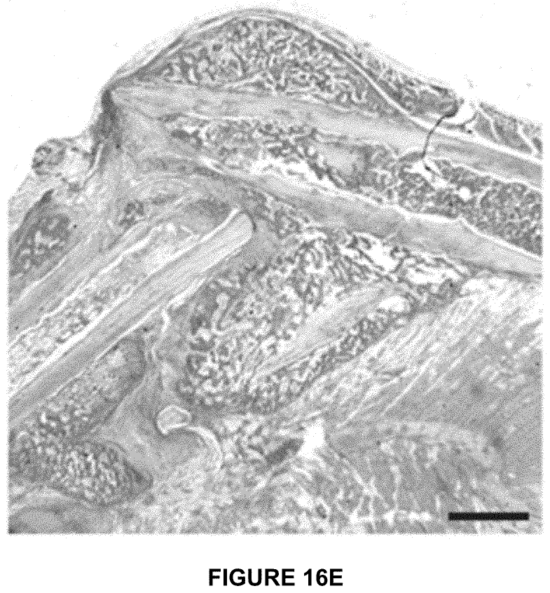

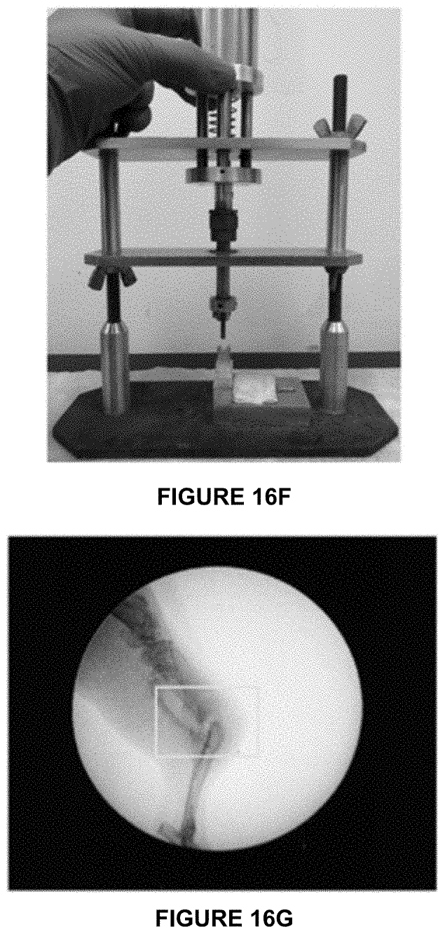

The data thus far has demonstrated that PEGDMA microrods can be loaded with β-NGF and β-NGF is bioactive and released over a 7-day period. Given these findings, it was hypothesized that sustained release of β-NGF loaded PEGDMA microrods could accelerate endochondral fracture repair. To test this hypothesis, a murine model of long bone healing was utilized, wherein closed, mid-shaft fractures were created in the right tibia of adult wild type mice (FIGS. 16F, 16G). These non-stabilized fractures have previously been shown to incite robust endochondral repair (Bahney, et al., 2014, J Bone Mineral Res 29(5)). Per a previous study, NGF was most effective in promoting fracture repair when delivered 7-days post injury, during the cartilaginous phase of bone healing (Rivera, et al., 2020, Sci Reports 10:22241). Thus, PEGDMA microrods were delivered 7-days post injury using a Hamilton syringe for percutaneous directly to the fracture site. First, the 16,000 microrods suspended in 20 μL saline (lightly stained blue) were shown to be effectively delivered to fracture callus (FIGS. 16A-16D) and remained localized throughout the entirety of the repair period of 14 days (FIGS. 16C-16E) as visualized by Hall Brunt's Quadruble (HBQ) staining (cartilage=blue, bone=red).

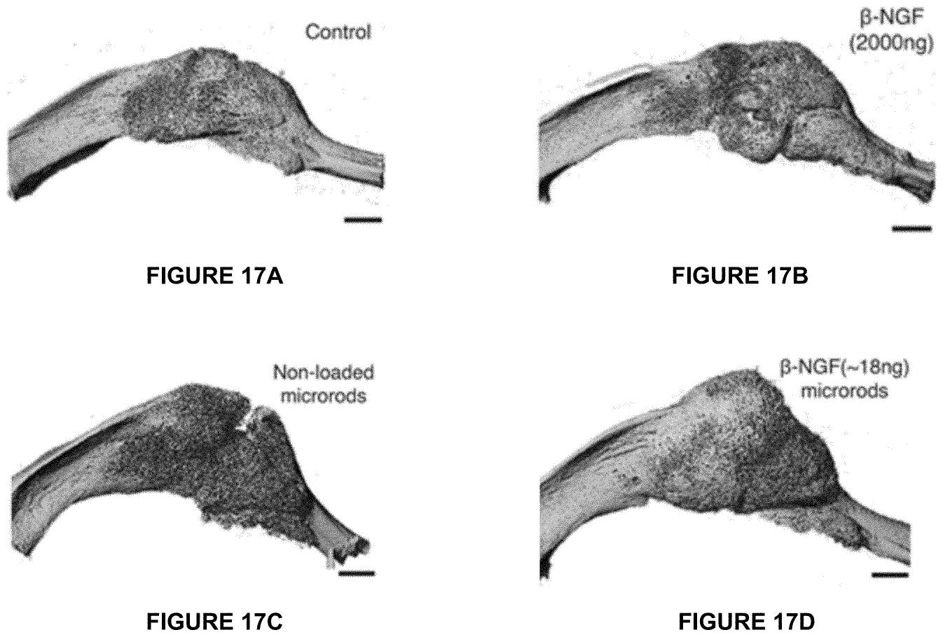

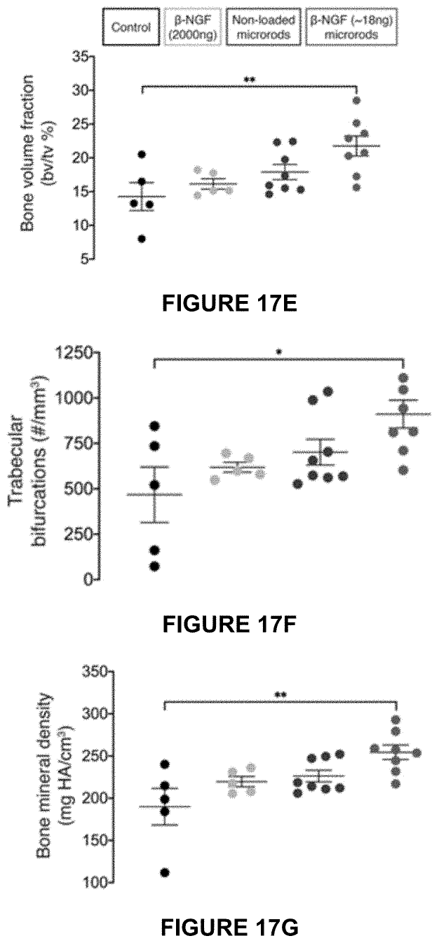

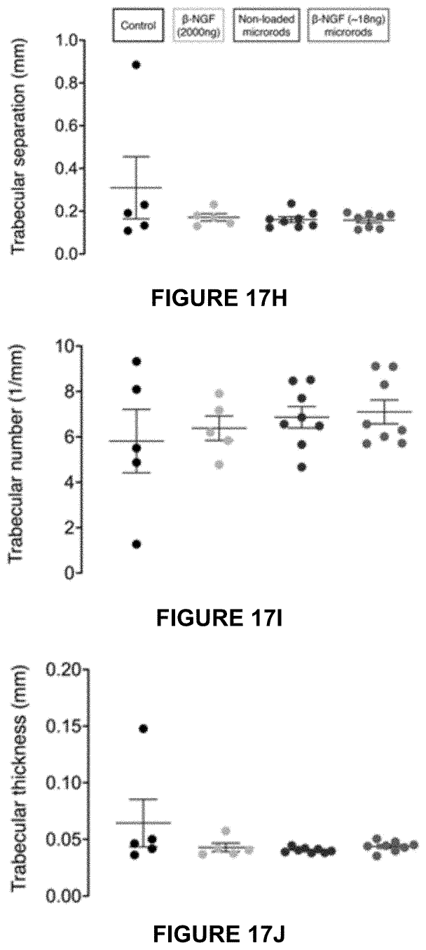

To assess the effectiveness of β-NGF, 16,000 microrods containing 18 ng of β-NGF was injected into the fracture callus. For comparison, additional mice were divided into three experimental (injection) groups: fracture calluses were percutaneously injected with either 20 μL saline, single dose of β-NGF (2000 ng), or non-loaded PEGDMA microrods. All percutaneous injections were administered 7 days post-fracture and were allowed to heal for 7 days (14 days post-fracture), at which point the calluses were harvested for Micro-Computed tomography (μCT) analysis to quantify the mineralized tissue within the fracture callus and to analyze the bone tissue microarchitecture. By gross examination of the images, the β-NGF microrods group appeared to have a largest most consolidated bony callus compared to all others (FIGS. 17A-17D). Quantification of the bone volume fraction (BFV) confirmed the highest BVF in the β-NGF microrods group with a significantly higher BVF (˜52% increase) compared to saline controls (FIG. 17E). Additionally, the fractures treated with β-NGF microrods resulted in more mature fracture calluses. β-NGF microrods treatment significantly increased trabecular bifurcations (TB, ˜95% increase) and bone mineral density (BMD, ˜34% increase) compared to saline controls (FIGS. 17F, 17G). MicroCT analysis of trabecular bone within the fracture callus is further shown in FIGS. 17H-17J. Interestingly, the soluble β-NGF did not improve bone formation to the same extent as β-NGF microrods and was not statistically different from the saline controls. Although not statistically different, the non-loaded microrods exhibit higher amounts of BVF, TB, and BMD when compared to the soluble NGF and saline treated groups.

### 4.5 B-NGF Loaded PEGDMA Microrods Reduce Cartilaginous Tissue Volume in the Fracture Callus

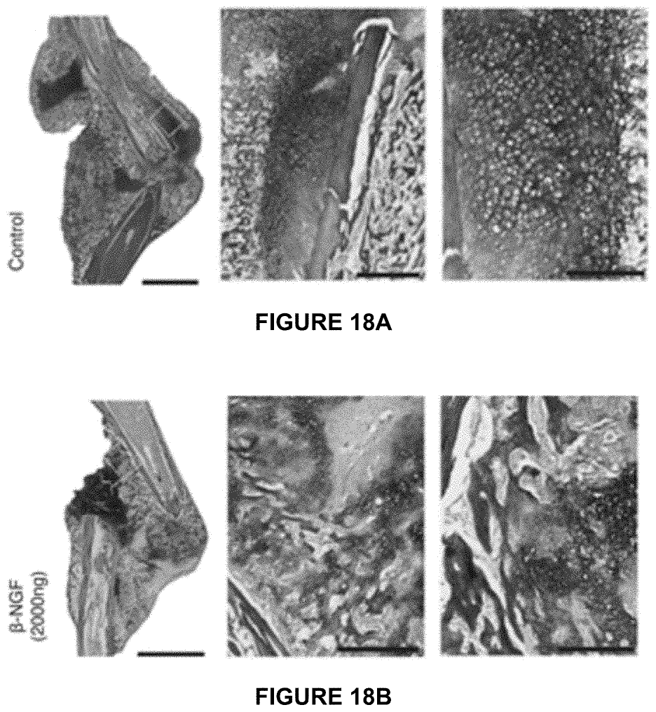

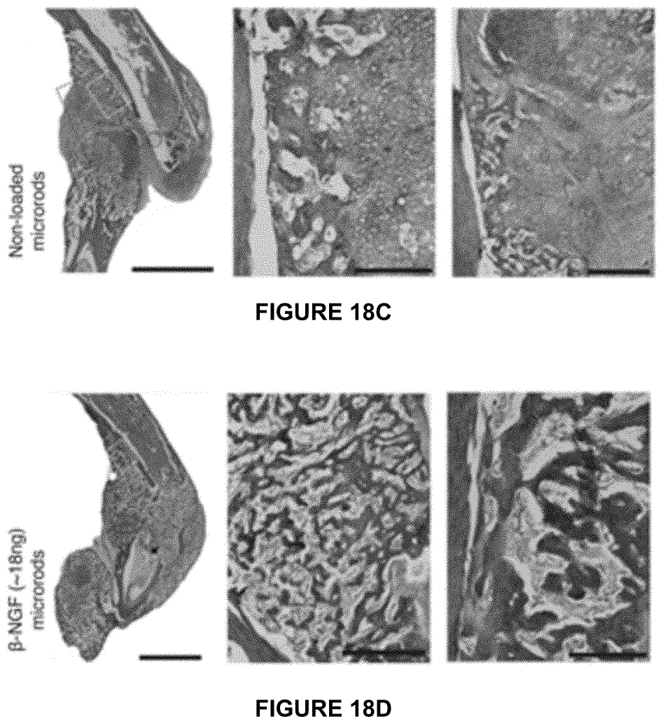

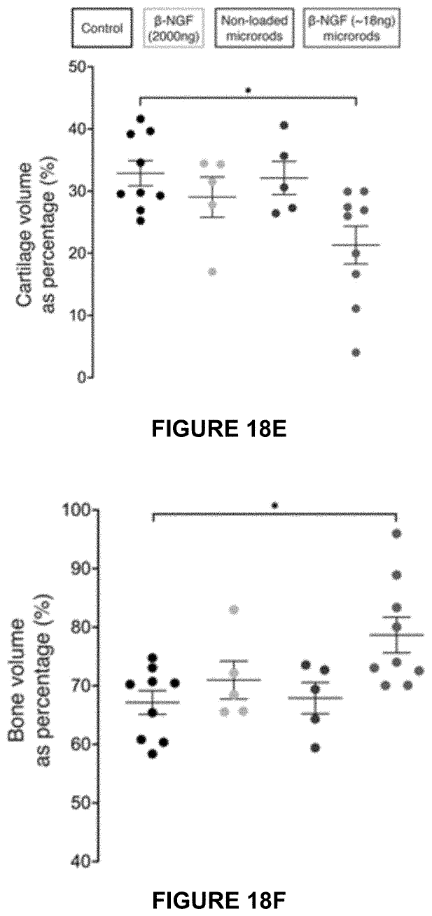

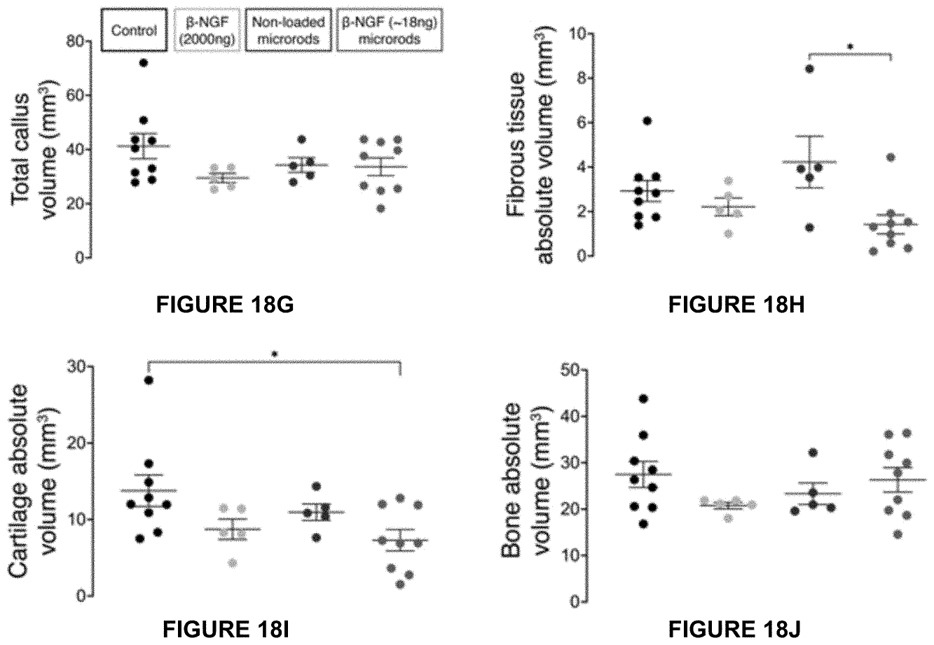

To understand the differences noted by μCT at a more detailed tissue level, quantitative histology was employed to differentiate the cartilage and bone fractions within the fracture callus. Histological images of tibia sections harvested 14 days post-fracture stained with HBQ (cartilage=blue, bone=red) visually indicate that β-NGF microrods have the highest amount of bone (FIGS. 18A-18D). Saline-treated fracture calluses had the high quantities of cartilage as percent composition of the callus (32+/−2%) with the least bone volume as percent composition (67+/−2%) compared to other treatment groups indicating the least advanced healing (FIGS. 18A-18F). Higher magnification images verify large proportions of chondrocytes in the fracture callus, which suggest the fracture is only nearing the cartilage to bone transition phase in endochondral repair (FIG. 18A). Near identical results were observed for the empty microrod treated fracture calluses with cartilage and bone volume at 32+/−2.7% and 68+/−2.7%, respectively (FIGS. 18C, 18E, and 18F). The soluble NGF-treated fracture calluses resulted in slightly elevated levels of bone (71+/−3.2%) and lowered cartilage volumes (29+/−3.2%) relative to the empty microrods and untreated controls, but this effect was not statistically different. β-NGF loaded microrods were the only treatment group to significantly change the fracture callus composition producing robust bone formation (79+/−3%) (FIGS. 18D and 18F). β-NGF microrod-treated samples also show a significant visual reductions in cartilage and statistically different cartilage volume (21+/−3%) compared to saline controls (FIGS. 18D, 18E). Histomorphometric analyses of the fracture calluses are shown in FIGS. 18G-18J.

Thus, the trophic benefit of β-NGF therapy can be balanced, while minimizing its hyperalgesic effects, by providing sustained drug release at a dosing below this threshold of daily injections of 100 ng and above. To avoid repeated doses, PEGDMA microrods were used as a clinically relevant drug delivery platform. The majority of PEGDMA microparticle delivery platforms use spherical particles for bone repair applications (Sonnet, et al. 2013 J Orthopaed Res 31:1597-1604; Stukel, et al., 2015, J Biomed Materials Res Part A 103:604-613; Olabisi, et al., 2010, Tissue Engineering Part A 16:3727-3736). In the instant study, the use of high aspect ratio microrods for fracture repair is demonstrated, given that high aspect ratio particles have higher residence time and tend to evade phagocytosis or cellular internalization. The instant study is the first to use PEGDMA microrods for bone fracture repair.

By employing photolithography, PEGDMA microrods can be produced in a high throughput fashion. Moreover, β-NGF loading onto PEGDMA microrods was increased using 90% (v/v) PEGDMA macromer. Finally, micrographs confirmed that molecules of smaller size can more readily diffuse across or into the PEGDMA polymer mesh network.

The loaded β-NGF retained its bioactivity, as demonstrated with an in vitro proliferation assay using the TrkA expressing TF-1 cell line, performed with 16,000 PEGDMA microrods, versus 100,000 used for the loading assay (because only 16,000 could effectively be aspirated in a 20 μL syringe used for the in vivo experiments). It was calculated that approximately 30-40% of total protein loaded in 100,000 microrods was about 1-2 mg. Thus, the highest calculation of 2 μg (2000 ng) was loaded into the 16,000 microrods and set that as the soluble NGF amount for all experiments in parallel. Some of the β-NGF proteins may lose their native molecular arrangement during loading or elution, thus reducing bioactivity. Nonetheless, 16,000 PEGDMA microrods containing 18 ng of bioactive β-NGF's had a potent effect on TF-1 cell proliferation, likely driven by the sustained release of β-NGF over the 96-hour experimental period. A nominal increase in proliferation of cells cultured with non-loaded PEGDMA microrods was also observed.

Upon examining PEGDMA microrod localization during endochondral fracture repair in a murine fracture model, it was possible to histologically localize the PEGDMA microrods at both 5- and 7-days post-injection. However, the PEGDMA microrods were no longer visible after 14 days, suggesting that the PEGDMA microrods are perhaps physically degraded over time. Degradation products of the PEGDMA microrods are not of concern in vivo based on established biocompatibility and non-cytotoxicity. And efficacy studies confirm that the PEGDMA microrods were localized in the fracture callus long enough to deliver the therapeutic payload and, therefore, are suitable for application in fracture callus injections of β-NGF.

Therapeutic efficacy of β-NGF delivery via PEGDMA microrods was validated using non-stabilized tibial fracture in mice followed by μCT analysis and quantitative histomorphometry. β-NGF loaded microrods enhanced endochondral fracture repair, as evidenced by the reduction in cartilage volume and statistically significant increases in bone volume fraction (BVF), trabecular bifurcations (TB), and bone mineral density (BMD). Furthermore, the woven-like bone morphology and minimal hypertrophic chondrocyte cells within the fracture callus indicates a quicker transition into the bony callus formation after injection with sustained release β-NGF loaded PEGDMA microrods, likely due to the sustained release of β-NGF from the PEGDMA microrods versus a large bolus dose from free/injected β-NGF. Large bolus doses are at risk of off-target effects and toxicity.

In addition to potentially extending the half-life of β-NGF, the high localization of β-NGF provided by the PEGDMA microrods may synergistically have contributed to the robust endochondral fracture response seen in mice treated with β-NGF loaded microrods.

### Example 5. Nanowires

### 5.1 Nanowire Fabrication

In an effort to test a clinically relevant drug delivery platform for sustained and local delivery of NGF^{R100W} to fractures via percutaneous injection of functionalized nanowires, PCL-nanowires are coated with heparin for affinity binding of the painless NGF and then use layer-by-layer (LbL) electrostatic coating to tune the release kinetics. Bioactivity of the nanowire-released NGF^{R100W} is verified in vitro using established cell proliferation assays. Following optimization of release kinetics in vitro, efficacy of the NGF^{R100W}-nanowires is tested in fractured wild-type or diabetic mice. Injectable NGF^{R100W}-nanowires are expected to accelerate endochondral repair through sustained and local delivery of NGF^{R100W}.

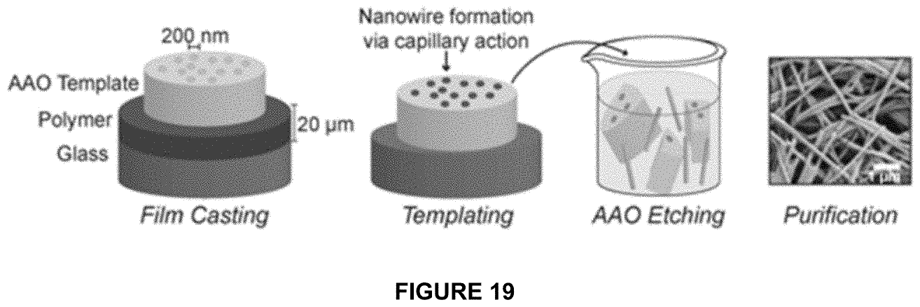

Nanowires are fabricated from polycaprolactone (PCL) polymers using a nano-templating technique (FIG. 19). PCL has distinct advantages for biomedical applications, as the polymer is biodegradable and nonimmunogenic. An anodized aluminum oxide (AAO) substrate with controlled pore size is used as the template for nanowire formation (Zamecnik, et al., 2017, ACS Nano 11:11433-11440). A PCL film is cast onto a glass substrate and heated to above melting temperature while in contact with the AAO template. This causes rapid nanowire formation into the AAO pores via capillary action. Upon templating and cooling of the polymer material, nanowires are purified by membrane detachment and selective AAO etching with sodium hydroxide. The nanowire width is controlled by the pore size of the AAO mold, while the length of the nanowires can be tuned by varying the thickness of the polymer film. Nanowires with lengths ranging from 2-20 μm are fabricated, and a width of 200 nm is consistently used thus far. The PCL nanowires are functionalized via incorporation of cargo into the polymeric backbone, such as with hydrophobic fluorescent dyes for in vitro and in vivo visualization of the nanowires.

5.2 Nanowire Functionalization with NGF for Controlled Delivery

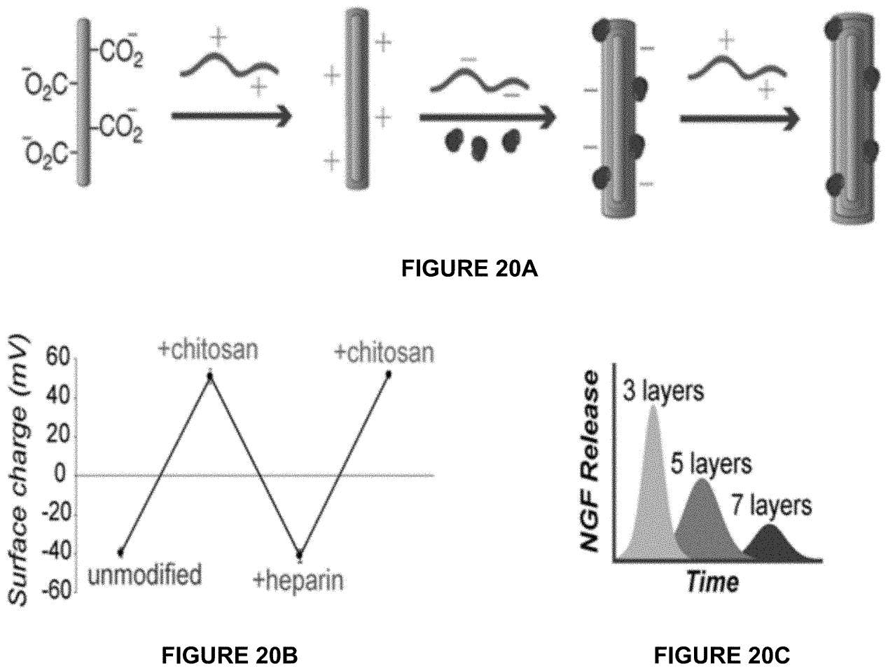

In order to attach the painless NGF growth factor cargo to nanowire scaffolds, a layer-by-layer (LbL) electrostatic assembly approach was utilized (Zamecnik, et al., 2017, ACS Nano 11:11433-11440) (FIG. 20A). LbL assembly has been used extensively for drug delivery applications and affords a facile and modular means of attaching biological cargo onto nanomaterials, such that increasing layers will increase growth factor retention (FIG. 20C). The PCL nanowires bear a strong negative charge as a result of the alkaline etching method used in the fabrication process. This negative charge allowed electrostatic assembly of biopolymers onto the surface of the nanowires. Chitosan (positive charge) and heparin (negative charge) were chosen for LbL assembly due to their biocompatibility and the growth factor affinity of heparin. Both chitosan and heparin have been successfully deposited onto the surface of the nanowires, as determined by zeta potential measurements of nanowire surface charge (FIG. 20B). Multiple layers were deposited, resulting in observed charge oscillation between positively charged (chitosan), and negatively charged (heparin-coated) nanowires. In addition to their charge, chitosan is also advantageous due to it antimicrobial properties (Jiang, et al., 2014, Natural and Synthetic Biomedical Polymers ch. 5:91-113), and heparin shows high affinity for multiple growth factors including NGF (Martino, et al., 2013, PNAS USA 110:4563-4568; Hu, et al., 2020, J Cell Mol Med 24:8166-8178).

### 5.3 Determination of Painless NGF Adsorption Efficiency and Release Kinetics

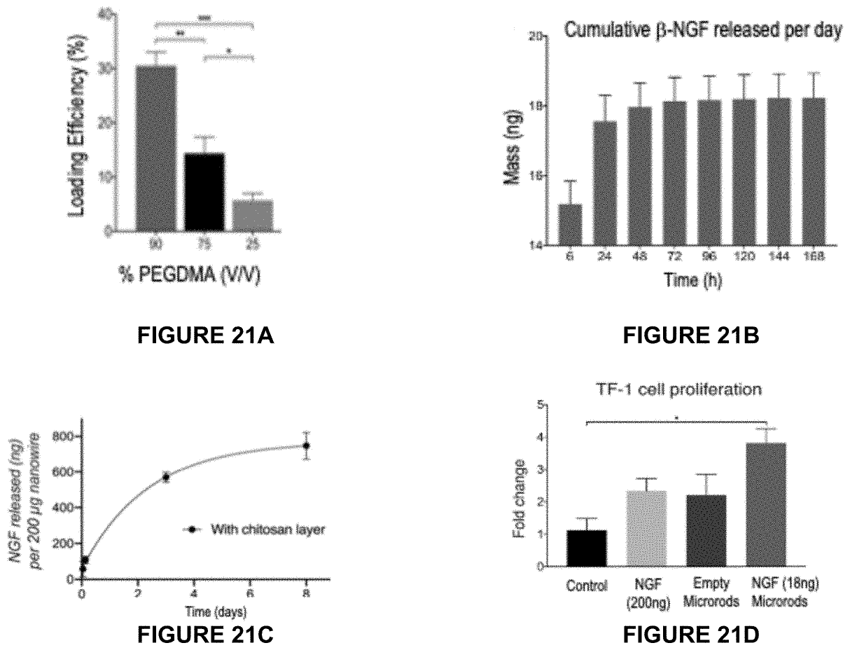

Using the poly(ethylene) glycol dimethylacrylate (PEGDM) microrods (15×100 mm) for the controlled release of NGF, adsorption efficiency of the PEGDM could be modified by changing the concentration of the monomer (FIG. 21A). The lyophilized PEDMA at a 90% (v/v) concentration could load 20 ng of NGF and demonstrated controlled release (FIG. 21B). A move was made from the PEGDM microrods to the PCL nanowires, due to the ability to further tune NGF release and their nanoscale (200 nm wide×20 μm long). To load NGF onto the nanowires, NGF is solubilized in pH 6 sodium acetate buffer with heparin in a 1:2 molar ratio. The negatively charged heparin then anchors the NGF onto the positively charged chitosan nanowires. Using the nanowire system described herein, 5 μg of NGF (250 times more than with microrods) have been successfully loaded with upwards of 75% efficiency. Sustained first order release of the NGF was achieved over 8 days (FIG. 21C). These data were generated using only a single layer of chitosan; further tuning of the NGF release kinetics to become more linear can be achieved by adding multiple layers of the electrostatic polymers (Zamecnik, et al., 2017, ACS Nano 11:11433-11440; Woodruff and Hutmacher, 2010, The return of a forgotten polymer—Polycaprolactone in the 21^{st} century 35:1217-1256; Xue, et al., 2013, Biomaterials 34:2624-2631). Release kinetics were determined using mBCA assay. To add rigor to these preliminary data, specificity of the protein release is confirmed using NGF-ELISAs.

### 5.4 In Vitro Bioactivity of NGF^{R100W}-Nanowires

In addition to characterizing the release kinetics, it was verified that the NGF released from the nanowires maintains bioactivity. The canonical bioactivity test for NGF is the TF1 erythroblast cell proliferation assay (Chevalier, et al., 1994 Blood 83:1479-1485). NGF- and NGF^{R100W}-nanowires at varying concentrations are incubated with TF1 erythroblasts over 3-5 days, and cell proliferation are quantified using CyQuant or PrestoBlue Assay and compared to proliferation rates with soluble NGF/NGF^{R100W} and empty chitosan-nanowire controls. NGF retains bioactivity following the release from the PEGDMA microrods (FIG. 21D). In addition to measuring TF1 cell proliferation, NGF/TrkA pathway activation in the cells is quantified by measuring cFOS by qRT-PCR and phospho-TrkA/AKT/Erk/PLCg by Western Blot at 1-, 24- and 48-hours following treatment (Sung, et al., 2018, J Neurosci 38:3394-3413; Yang, et al., 2020, Prog Neurobiol 194:101866).

### 5.5 In Vivo Evaluation of NGF-Nanowires in Normal Fracture Healing

Therapeutic efficacy of NGF-nanowires is assessed using the established murine tibial fracture model detailed above. Preliminary data indicate that optimal healing occurs when NGF injections began 7-days post fracture during the endochondral phase of fracture healing (data not shown). Next, systemic (intraperitoneal) NGF^{R100W} are compared to six treatment groups injected percutaneously into the callus at day 7 post-fracture: (i) PBS control, (ii) soluble NGF (single injection), (iii) soluble NGF^{R100W} (single injection), (iv) empty nanowires, (v) NGF-nanowires, (vi) NGF^{R100W}-nanowires. Injections into the fracture callus are guided by fluoroscopy and precise volume delivery achieved using a Hamilton syringe system. In preliminary studies, effective injection and identification of the nanomaterials are demonstrated near the fracture site 7-days post-injection (data not shown). Further, local, and sustained delivery of NGF from these nanomaterials leads to increased bone quality in the fracture callus 14 days after fracture, as measured by quantitative μCT when compared to a single injection of NGF (data not shown).

Dosing and timing of NGF delivery are standardized to 2.5 μg NGF/NGF^{R100W}, corresponding to 0.5 μg NGF per day for 5 days, delivered 7 days post-fracture. This loading value can be adjusted as necessary, however, efficacy of this dose and the ability to load more than this required amount of NGF onto the nanowires utilizing the LBL technique are demonstrated. Empty nanowire dose is standardized to the injected dose of NGF-nanowires. Tibia is harvested to access bone healing at 10, 14, 21, and 28 days post-fracture. Pain sensation, functional testing of fracture healing and biomarker analysis are completed as described above. Histomorphometry is the primary success criteria. Mean and standard deviation indicate that only 8 mice are needed to reach 80% power (a=0.05, calculated in GPower*); to maintain consistency, a sample size planning of N=10 is selected, allowing for the potential of increased variation.

### Example 6. Biomarker-Based Quantification of Fracture Healing

### Fracture Biomarker

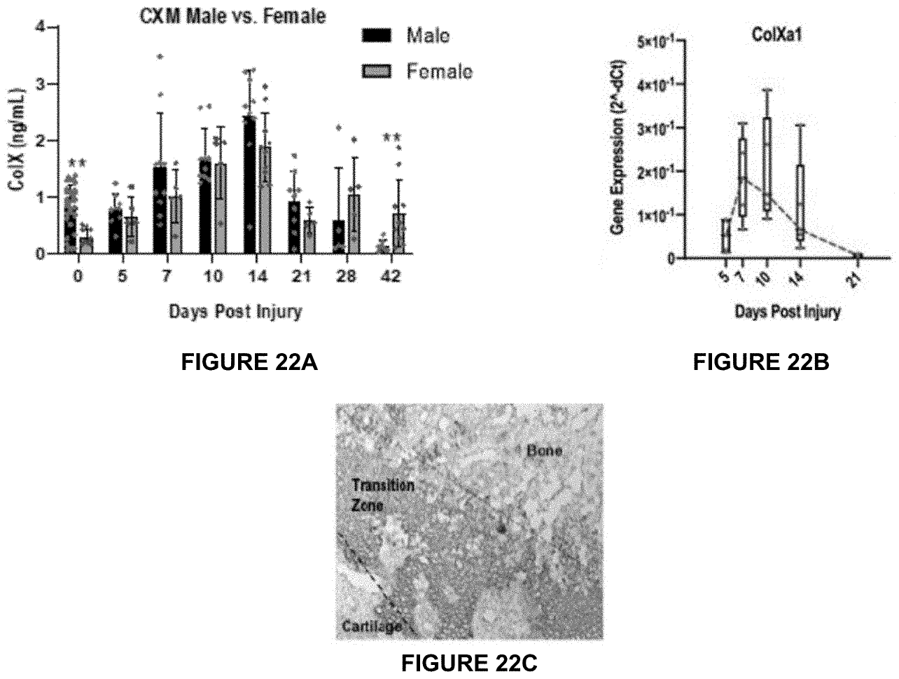

The collagen X (“Cxm”) biomarker is the canonical marker of chondrocyte hypertrophy and is transiently expressed as cartilage turns into bone (FIG. 1). Cxm levels were correlated to collagen X gene expression and immunohistochemistry in fracture healing 1 (FIGS. 22A-22C). This serum bioassay is a novel, non-destructive longitudinal measurement of the biology at the fracture callus and allows the comparison of molecular signatures of chondrocyte hypertrophy in control vs NGF treated mice. Blood is collected from the tail vein (˜25 μl, nondestructive) 3 days prior to and 14 days following fracture, and then via cardiac punch post-euthanasia at the terminal time point of the study. Blood is saved as serum for batch testing.

Painless NGF (NGF^{R100W}) is thus established as a novel therapeutic for accelerating endochondral bone repair. To support therapeutic development, the timing and dose of NGF^{R100W} that accelerates fracture healing but minimizes nociception are determined. Fracture healing outcomes are rigorously evaluated using the above-described techniques of histomorphometry, quantitative μCT, and mechanical testing, as well as the Cxm biomarker.

In an effort to understand why NGF/NGF^{R100W} is more effective later (endochondral phase) rather than earlier delivery, differential gene expression is determined using RNAseq, with the expectation that certain osteogenic pathways would be more significantly upregulated with later delivery, as well as anti-apoptotic and proliferative pathways based on preliminary data. A 1:1 efficacy of the wild type to painless NGF is assumed.

### Example 7. Genetic Knock-Out of TrkA Receptor to Test Role in Endochondral Fracture Repair

In order to determine the extent to which NGF-TrkA signaling is required for endochondral fracture repair by conditionally knocking-out (KO) the TrkA receptor in either chondrocytes specifically, or in all cells, during fracture repair, TrkAfl/fl mice (Tomlinson, et al., 2017, PNAS USA 114:E3632-E3641) are crossed to either the chondrocyte specific (00110) or global (R26) tamoxifen inducible Cre-drivers, and the resultant mice are treated with tamoxifen from days 6-10 (Wong, et al., 2020, bioRxiv 986141; Hu, et al., 2017, Development 144:221-234). The Col10CreERT mouse is used, because it is specific to the hypertrophic chondrocytes. Fracture healing in the Col10CreERT::TrkAfl/fl and R26CreERT2::TrkAfl/fl mice is compared to tamoxifen-treated wild type mice using the standard outcomes detailed above (histomorphometry, μCT, Cxm biomarker, biomechanics). This directly tests whether the TrkA receptor is critical to endogenous fracture healing and whether its signaling function acts primarily through the hypertrophic chondrocytes or another cell population.

In order to validate that, in long bone fracture healing, the painless and wild type NGF are acting through the TrkA receptor, the Col10CreERT::TrkAfl/fl and R26CreERT2::TrkAfl/fl are given therapeutic NGF^{R100W} or NGF during the endochondral phase of healing (d7-9), and qRT-PCR and RNAseq analyses are used, as described above, to determine how gene expression patterns are differentially affected. The NGFs likely stimulate fracture repair through the TrkA receptor in chondrocytes, such that, cFOS and endochondral gene expression are significantly down regulated in the KO animals as compared to wild type mice. NGF/NGF^{R100W} therapy likely cannot rescue the effect of the KO, indicating the minor NGF receptors do not contribute too significantly to the trophic actions of NGF/NGF^{R100W}.

### Example 8. In Vivo Evaluation of NGF-Nanowires in Delayed Fracture Healing

In order to evaluate the potential of the therapy to work in a scenario of delayed fracture healing, a co-morbidity driven delayed union is also analyzed. Fractures are made as described above, but the Lepob diabetic mouse (Jackson, B6.Cg-Lepob/J) or a murine model of aging or osteoporosis is used instead of C57Bl6/J Wild Type (Roszer, et al., 2014, Cell Tissue Res 356:195-206; Gao, et al., 2018, Orthop Surg Res 13:145; Khan, et al., 2013, J orthopaed trauma 27:656-662). The diabetic mouse is chosen, since diabetes is a well-established co-morbidity associated with poor fracture healing due to reduced vascular flow and a sustained systemic pro-inflammatory state. Furthermore, the Lepob mice are on the same B6/J background as the Wild Type, making comparison between these two strains possible.

As described earlier, six (6) experimental groups are injected percutaneously into the fracture callus of the Lepob mice or a murine model of aging or osteoporosis at day 7 post-fracture, including (i) PBS control, (ii) soluble NGF, (iii) soluble NGF^{R100W}, (iv) empty nanowires, (v) NGF-nanowires and (vi) NGF^{R100W}-nanowires. In order to reduce overall animal numbers, healing of the Lepob mice is only compared to wild type at day 14 following repair, because this early time point is the point of maximal difference fracture callus composition. The additional time points of 21 and 28 days can be added, if any differences in healing patterns with the nanowires are to arise. Pain sensation, functional testing of fracture healing, and biomarker analysis are completed as described above, with the variation, power, and success criteria assumed to be the same as in the previous example. Fracture healing outcomes from the diabetic mice are compared to the normal healing from the previous example. To ensure that there is not a different mechanism of action in Lepob mice compared to Wild Type, an additional 5 mice/group for each genotype are harvested for quantitative RT-PCR analysis 14 days after fracture as described above, with assessment of the key pathways determined.

Thus, nanowires serve as an injectable and biocompatible scaffold material for sustained and local delivery of painless NGF to accelerate fracture repair in both the normal and delayed murine fracture models. In certain embodiments, the nanowires produce equivalent, if not better, functional outcomes in fracture repair compared to soluble NGF delivery. The preliminary data, including that shown in FIGS. 21A-21D, indicate that sufficient painless and wild type NGF can be efficiently loaded to the nanowires to achieve controlled release over the 5-day time period by tuning the LBL platform. If heparin interacts with different signaling proteins and sequesters unwanted endogenous proteins to modify downstream effects, in certain embodiments, pre-incubation of heparin and NGF is performed at a 1:1 ratio to limit the amount of unbound heparin, or other negatively charged polymers such as poly(glutamic acid) or poly(acrylic acid) are used for subsequent assembly layers. In another embodiment, a method of immobilization is employed in which NGF-binding peptides or antibodies are covalently conjugated onto the nanowires for growth factor loading. In still another embodiment, heparin-coated nanowires, without growth factor bound, can be added as an additional group to this and the previous example.

The present disclosure is not to be limited in scope by the specific embodiments described herein. Indeed, various modifications of the disclosure in addition to those described herein will become apparent to those skilled in the art from the foregoing description and the accompanying figures. Such modifications are intended to fall within the scope of the appended claims.

What is claimed is:

1. A method for stimulating bone healing in a subject, accelerating bone healing in a subject, and/or improving bone healing in a subject, comprising administering a pharmaceutical composition to the subject, wherein the composition comprises nerve growth factor (NGF).

2. A method for stimulating bone healing in a subject, accelerating bone healing in a subject, and/or improving bone healing in a subject, comprising administering a pharmaceutical composition to the subject, wherein the composition comprises biomaterial carriers comprising nerve growth factor (NGF).

3. The method of claim 1 or 2, wherein the bone healing is bone fracture healing.

4. The method of any one of claims 1-3, wherein the NGF is a mutant NGF.

5. The method of claim 4, wherein the NGF has a mutation at amino acid 100 of the mature NGF protein.

6. The method of claim 4 or 5, wherein the NGF is NGF^{R100W}.

7. The method of any one of claims 1-6, wherein a conversion of cartilage to bone is promoted in the subject.

8. The method of any one of claims 2-7, wherein the biomaterial carriers are biocompatible.

9. The method of any one of claims 2-8, wherein the biomaterial carriers are biodegradable.

10. The method of any one of claims 2-9, wherein the biomaterial carriers are selected from the group consisting of nanowires, nanotubes, nanorods, microwires, microtubes, and microrods.

11. The method of any one of claims 2-10, wherein the biomaterial carriers are microrods.

12. The method of any one of claims 2-10, wherein the biomaterial carriers are nanowires.

13. The method of claim 12, wherein the nanowires are coated with heparin.

14. The method of any one of claims 1-13, wherein the composition is administered by subcutaneous or percutaneous injection.

15. The method of any one of claims 1-14, wherein the administration is local.

16. The method of any one of claims 4-15, wherein bone formation is increased in a fracture.

17. The method of any one of claims 1-16, wherein the bone healing is endochondral.

18. The method of any one of claims 1-17, wherein the subject has normal bone healing.

19. The method of any one of claims 1-17, wherein the subject has delayed or non-union bone healing.

20. The method of any one of claims 1-19, wherein serum collagen X (Cxm) expression is earlier and/or increased upon administration of the composition.

21. The method of any one of claims 1-20, wherein NGF-associated nociception is minimized.

22. The method of any one of claims 1-21, wherein the composition is administered during the endochondral or cartilaginous phase of bone healing.

23. The method of any one of claims 3-21, wherein the composition is administered between about two months and about three months post-fracture.

24. The method of any one of claims 1-23, wherein the subject has a fracture in a bone that heals through secondary healing or endochondral repair.

25. The method of any one of claims 1-24, wherein the subject has a long bone fracture.

26. The method of any one of claims 1-25, wherein newly formed bone contains higher trabecular number, connective density, and/or bone mineral density.

27. The method of any one of claims 1-26, wherein cartilage volume in the subject decreases, and bone volume in the subject increases upon administration of the composition.

28. A pharmaceutical composition comprising i) nerve growth factor (NGF) and ii) a pharmaceutically acceptable carrier for use in stimulating bone healing in a subject, accelerating bone healing in a subject, and/or improving bone healing in a subject.

29. A pharmaceutical composition comprising i) biomaterial carriers comprising nerve growth factor (NGF) and ii) a pharmaceutically acceptable carrier for use in stimulating bone healing in a subject, accelerating bone healing in a subject, and/or improving bone healing in a subject.

30. A pharmaceutical composition comprising i) nerve growth factor (NGF) and ii) a pharmaceutically acceptable carrier for use in treating bone fracture in a subject.

31. A pharmaceutical composition comprising i) biomaterial carriers comprising nerve growth factor (NGF) and ii) a pharmaceutically acceptable carrier for use in treating bone fracture in a subject.

32. The composition of any one of claims 28-31, wherein the NGF is a mutant NGF.

33. The composition of claim 32, wherein the NGF has a mutation at amino acid 100 of the mature NGF protein.

34. The composition of claim 32 or 33, wherein the NGF is NGF^{R100W}.

35. The composition of any one of claims 29 and 31-34, wherein the biomaterial carriers are biocompatible.

36. The composition of any one of claims 29 and 31-35, wherein the biomaterial carriers are biodegradable.

37. The composition of any one of claims 29 and 31-36, wherein the biomaterial carriers are selected from the group consisting of nanowires, nanotubes, nanorods, microwires, microtubes, and microrods.

38. The composition of any one of claims 29 and 31-37, wherein the biomaterial carriers are microrods.

39. The composition of any one of claims 29 and 31-37, wherein the biomaterial carriers are nanowires.

40. The composition of claim 39, wherein the nanowires are coated with heparin.

41. The composition of any one of claims 28, 29, and 32-40, wherein the bone healing is bone fracture healing.

42. The composition of any one of claims 28-41, wherein the composition is administered to the subject by subcutaneous or percutaneous injection.

43. The composition of claim 42, wherein the administration is local.

44. The composition of any one of claims 28, 29, and 32-43, wherein the bone healing is endochondral.

45. The composition of any one of claims 28-44, wherein the subject has normal bone healing.

46. The composition of any one of claims 28-44, wherein the subject has delayed or non-union bone healing.

47. The composition of any one of claims 28-46, wherein the composition is administered to the subject during the endochondral/cartilaginous phase of bone healing.

48. The composition of any one of claims 28-46, wherein the composition is administered to the subject between about two months and about three months post-fracture.

49. The composition of any one of claims 28-48, wherein the subject has a fracture in a bone that heals through secondary healing or endochondral repair.

50. The composition of any one of claims 28-49, wherein the subject has a long bone fracture.


SEQUENCE LISTING (4 sequences)

SEQ ID NO: 1 - protein, 241 aa, Homo sapiens
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 2 - protein, 241 aa, Artificial Sequence
  source: /note=“Description of Artificial Sequence Synthetic polypeptide”
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWWFIRIDTACVCVLSRKAVRRA

SEQ ID NO: 3 - protein, 240 aa, Mus musculus
MSMLFYTLITAFLIGVQAEPYTDSNVPEGDSVPEAHWTKLQHSLDTALRRARSAPTAPIAARVTGQTRNITVDPRLFKKRRLHSPRVLFSTQPPPTSSDTLDLDFQAHGTIPFNRTHRSKRSSTHPVFHMGEFSVCDSVSVWVGDKTTATDIKGKEVTVLAEVNINNSVFRQYFFETKCRASNPVESGCRGIDSKHWNSYCTTTHTFVKALTTDEKQAAWRFIRIDTACVCVLSRKATRR

SEQ ID NO: 4 - protein, 240 aa, Artificial Sequence
  source: /note=“Description of Artificial Sequence Synthetic polypeptide”
MSMLFYTLITAFLIGVQAEPYTDSNVPEGDSVPEAHWTKLQHSLDTALRRARSAPTAPIAARVTGQTRNITVDPRLFKKRRLHSPRVLFSTQPPPTSSDTLDLDFQAHGTIPFNRTHRSKRSSTHPVFHMGEFSVCDSVSVWVGDKTTATDIKGKEVTVLAEVNINNSVFRQYFFETKCRASNPVESGCRGIDSKHWNSYCTTTHTFVKALTTDEKQAAWWFIRIDTACVCVLSRKATRR